(19) United States
(12) Patent Application Publication
(10) Pub. No.: US 2015/0359644 A1
(43) Pub. Date: Dec. 17, 2015

(54) Systems, Devices, and Methods for Prosthetic Socket Adjustment

(71) Applicant: University of Washington Through Its Center for Commercialization, Seattle, WA (US)

(72) Inventors: Joan E. Sanders, Sammamish, WA (US); John Cagle, Everett, WA (US); Morgan Redfield, Seattle, WA (US); Christian B. Redd, Seattle, WA (US)

(21) Appl. No.: 14/832,097

(22) Filed: Aug. 21, 2015

Related U.S. Application Data
(63) Continuation of application No. PCT/US2014/017809, filed on Feb. 21, 2014.
(60) Provisional application No. 61/767,661, filed on Feb. 21, 2013.
(60) Provisional application No. 61/783,343, filed on Mar. 14, 2013.
(60) Provisional application No. 61/792,411, filed on Mar. 15, 2013.

(51) Int. Cl. A61F 2/80; A61F 2/76; A61F 2/78; A61F 2/68

(52) U.S. Cl. CPC A61F 2/80; A61F 2/68; A61F 2/76; A61F 2/7812; A61F 2002/802; A61F 2002/701

(57) ABSTRACT

The present invention generally relates to prosthetic socket accommodation systems. In some embodiments a prosthetic socket accommodation system may be configured to automatically adjust the accommodation system in response to user activities. In some embodiments, a controller of the prosthetic socket accommodation system may be customized for a prosthetics user based on an activity volume profile of the prosthetics user. The activity volume profile may correspond to a residual limb fluid volume response to prosthetics user activity. In some embodiments, a controller of a socket accommodation system may be configured to control pistoning during prosthetic socket use. Some embodiments are related to sensors for identifying prosthetics user activity. In some embodiments a three-axis sensor may be used to identify prosthetics user activity. In some embodiments a socket proximity sensor comprising an infrared sensor may be used to detect socket donning and doffing by the prosthetics user.

### CROSS-REFERENCE TO RELATED APPLICATIONS

The present application is a Continuation of PCT/US2014/017809 filed Feb. 21, 2014; which application claims priority to and the benefit of US Provisional Appln Nos. 61/767,661 filed on Feb. 21, 2013; 61/783,343 filed on Mar. 14, 2013; and 61/792,411 filed on Mar. 15, 2014; the contents of which are incorporated herein by reference.

### BACKGROUND OF THE INVENTION

The present invention generally relates to prosthetic devices. Some embodiments of the present invention relate to systems, methods, and devices for automatically adjusting accommodation devices of a prosthetic socket. Some embodiments of the present invention relate systems methods and devices for detecting prosthetic socket donning and doffing. Some embodiments of the present invention relate to systems, methods, and devices for detecting and characterizing prosthetic user activities.

People with limb amputation using a prosthesis face constant challenges as a result of size changes of their residual limb. The residual limb changes in size as a result of fluid volume fluctuations in the soft tissues (short term, e.g. days or weeks), as well as muscle bulk changes either atrophy or hypertrophy (long term, e.g. months or years). Both are clinically significant because limb volume changes cause the fit between the prosthetic socket and residual limb to change. When the residual limb reduces in volume, the socket may become too loose, leading to excessive pistoning, gait instability, and possibly a fall or injury. When the residual limb increases in volume, the socket may become tight and uncomfortable. Excessive interface stresses may restrict vascular flow, denying cells of nutrients and inducing soft tissue injury.

There are several methods for people with limb loss to accommodate for daily changes in the volume of their residual limb. The most common is to add or remove prosthetic socks. Adding or removing socks is simple and inexpensive to implement, but the method is inconvenient in that the person must remove the prosthesis and overlying clothing to effect the change. From clinical experience, some amputees spend 40 min or more a day putting on or taking off their prosthesis. Another method is to periodically doff the prosthesis to allow recovery of some of the limb volume lost earlier in the day. Doffing the prosthesis releases socket pressures on the limb and can be effective at facilitating limb volume recovery. But, like adding or removing socks, the prosthesis must be doffed which many people find inconvenient. Further, the person cannot ambulate during the doffing period unless supporting aides are used.

Because of these problems technologies that are flexible and allow easy adjustment of the socket are of interest. For example, a method using a vacuum within the prosthetic socket (termed “vacuum assist” or “elevated vacuum”), does not require the prosthesis be doffed. By applying a vacuum pressure to the socket, vacuum-assist devices are intended to pull residual limb soft tissues outwards during the swing phase of gait or during low weight-bearing conditions and help draw fluid into the residual limb, retarding daily fluid volume loss. Another method for accommodation is an adjustable socket that adds material to the inside of the socket. The primary technologies developed to add material to the inside of the socket include either air or liquid-filled bladders. Bladders have been developed to mount to the inside socket wall and to extend through holes in the socket to a pumping mechanism.

Several adjustable socket technologies are available or are emerging. These include air-inflatable inserts (e.g., Pneu-Fit™, Prosthetic Concepts, Little Rock, Ark.), liquid-filled bladders (e.g., Simbex's Active Contact System, Lebanon, N.H.), and magneto-rheological liquid systems. Most air-filled inserts operate effectively over only a narrow volume range, in part because air is compressible. Water-based solutions are essentially incompressible thus liquid-filled bladders do not have this limitation. Simbex's Active Contact System uses a passive mechanical control system to adjust volume of liquid-filled bladders. Other inventions extending from Simbex's method have been developed.

Further prosthetists, physicians, and prosthetics researchers are challenged to describe how persons with limb loss use their prostheses outside the clinic or laboratory. Performance tests such as the “timed up and go” test or the “six-minute walk” can be used to measure mobility of a prosthetic user in a clinic or laboratory, but information on what prosthesis users do in their daily lives can be difficult to acquire. Characterizing ways that prostheses are used is complicated by the range of situations and environments users encounter. The characterization of prosthesis use could be partially achieved by quantifying prosthetic wear (e.g., donning and doffing) and users' engagement in locomotor activities (e.g., walking and stair climbing) and fundamental body positions (e.g., standing or sitting). Accurate knowledge of prosthetic use in free living conditions would enhance prosthetic prescriptions, fitting processes, and measurement of outcomes.

Previous methods for measuring prosthetic use outside of a gait laboratory or clinic included self-report surveys and personal activity monitoring devices (e.g., pedometers and step activity monitors). Self-report surveys have been used to quantify frequency and duration of prosthetic use. However, self-report of activity among persons with limb loss has been noted to be unreliable when compared to a step-activity monitor. Pedometers and step-counters have been used to objectively measure step-activity of persons wearing prostheses over extended periods of time. While these sensors accurately measure gait activities, they are unable to provide information about body positions that may also be part of a person's habitual activity. Differentiation of body positions may be clinically important as sitting and standing can affect changes in residual limb volume and alter the fit of a prosthesis. Accurate knowledge of how much a prosthetic user sits or stands could thus be useful in determining changes in socket fit throughout the day.

Identification of activities and postures has previously been achieved through classification of data from one or more body-mounted sensors. This technique has been applied to characterize the quality of gait, discriminate activity levels, and determine body orientations of individuals without amputations. It has also been used on persons with lower limb amputations to quantify step counts, estimate ambulation time, and describe gait patterns. Algorithms have also been developed to identify locomotion and posture of individuals with an amputation from sensor data obtained over short time periods (i.e., up to several hours). While these studies demonstrated potential for activity and posture classification based on data from body-mounted sensors, there remain challenges to clinical use such as need for multiple sensors, subject donning requirements, low storage capacities, and short battery lives. Currently available sensors are also often restricted to short-term applications and/or require adherence to specific user protocols. Accordingly, more user-friendly and clinically-relevant solutions are needed to overcome these challenges.

While improvements to adjustable socket systems and prosthetic socket monitoring systems have been achieved, further improvements are desirable and can be made.

### SUMMARY OF THE INVENTION

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

In some embodiments, a method for customizing a prosthetics accommodation device for a residual limb of a prosthetics user is provided. The method may include the step of identifying an activity volume profile of the prosthetics user. The activity volume profile of the prosthetics user may correspond to a residual limb fluid volume response to prosthetics user activity. The method may further include the step of customizing a controller of the prosthetics accommodation device to provide customized automated accommodation for the prosthetics user based on the identified activity volume profile and based on detected prosthetics user activity.

In some embodiments, the prosthetics accommodation device may be a bladder accommodation device. In some embodiments, the bladder accommodation device is incorporated within a liner for the residual limb of the prosthetics user or within the socket or componentry. Optionally, the prosthetics accommodation device is a vacuum assist device. In some embodiments, the prosthetics accommodation device is incorporated within an insert placed into a prosthesis.

In some embodiments, the activity volume profile of the prosthetics user corresponds to the residual limb fluid volume response to prosthetics user walking, sitting, and standing. The residual limb fluid volume response may include residual limb fluid volume response to prosthetics user transitions between standing and sitting.

The activity volume profile may indicate that the prosthetic user loses fluid volume during walking and gains fluid volume during sitting. In such cases, the controller may be customized to provide prolonged socket pressure release during detected prosthetic user sitting and/or resting after walking. The prolonged socket pressure release may range from 3 minutes to 16 hours. When the activity volume profile indicates that the prosthetic user loses fluid volume during walking and gains fluid volume during stand-sit/sit-stand transitions, the controller may be customized to provide vacuum assist during detected prosthetic user walking Optionally, when the activity volume profile indicates that the prosthetic user gains fluid volume during walking, loses fluid volume during stand-sit/sit-stand transitions, and gains fluid volume during sitting, the controller may be customized to provide socket immediate and prolonged pressure release during detected prosthetic user sitting and/or resting after walking. The immediate pressure release may occur within 2 seconds to within 20 seconds of detection of prosthetic user sitting and/or resting after walking. The prolonged pressure release may range from 3 minutes to 16 hours. In some embodiments, the controller may be customized to provide prosthetic user alerts to reduce prolonged prosthetic user standing when the activity volume profile indicates that the prosthetic user gains fluid volume during walking, but experiences little volume change during resting (e.g., less than 1%). The alerts may be provided when the prosthetic user stands for more than 5 minutes. Optionally, the alerts may be provided more frequently (e.g., when the prosthetic user stands for a time ranging from more than 1 minute to more than 5 minutes).

In some embodiments, the prosthetics user activity may be detected by a single 3-axis accelerometer. Optionally, the prosthetics user activity of user donning and doffing the prosthetic may be detected by a socket proximity sensor. A socket proximity sensor may include one or more infrared distance sensors or ultrasonic distance sensors.

The controller may be further customized to automatically adjust the prosthetics accommodation device based on a position of the residual limb within the prosthetic socket. This may be beneficial to control the amount of pistoning experienced by the prosthetics user. The controller may compare an actual high position and an actual low position of the residual limb within the prosthetic socket to an upper target and a lower target in order to adjust the accommodation device. The controller may be configured to increase volume for the residual limb when the actual high position>upper target>actual low position>lower target. The controller may be configured to decreases pressure/volume for the residual limb with the accommodation device when the upper target>actual high position>lower target>actual low position. The position of the residual limb in the prosthetic socket may be indicated by signals from a force sensor (e.g., a piezoresistive force sensor) mounted in the bottom of the socket with an overlying low stiffness material like an open-cell foam.

In some embodiments the controller may be configured to operatively couple with a user input device. The user input device may be configured to send a signal to the controller indicating that the prosthetics user intends to stand and/or walk from a sitting position. The controller may be configured to adjust the prosthetics accommodation device in anticipation for the standing and/or walking of the prosthetics user.

In some embodiments, the prosthetics accommodation device comprises one or more pressure pulse sensors. The controller may be configured to be operatively coupled to the one or more pressure pulse sensors to adjust a fluid volume recovery strategy or one or more stress locations of the prosthetics accommodation device in response to the one or more pressure pulse sensors.

In another embodiment of the invention, a control system for use with a prosthetics accommodation device is provided that provides customized accommodation for a residual limb of a prosthetics user. The control system may include one or more sensors configured to generate signals indicative of prosthetics user activity. The control system may further include a controller operatively coupled to the one or more sensors and configured to be operatively coupled to the prosthetics accommodation device. The controller may be further configured to interpret the signals from the one or more sensors to determine current activities of the prosthetics user and to receive customization input. The customization input may be indicative of an activity volume profile of the prosthetics user. The activity volume profile of the prosthetics user may correspond to a residual limb fluid volume response to prosthetics user activity. The controller may be further configured to actuate the prosthetics accommodation device to provide customized accommodation for the prosthetics user based on the customization input and based on the signals from the one or more sensors that are indicative of prosthetics user activity.

The prosthetics accommodation device may be a bladder accommodation device. The bladder accommodation device may be incorporated within a liner for the residual limb of the prosthetics user, within an insert placed into the socket, or within the prosthetic socket or componentry. The prosthetics accommodation device may be a vacuum assist device. In some embodiments, the prosthetics accommodation device may be incorporated within an insert placed into the prosthesis.

In some embodiments, the activity volume profile of the prosthetics user may correspond to the residual limb fluid volume response to prosthetics user walking, sitting, and standing. The residual limb fluid volume response may further include residual limb fluid volume response to prosthetics user transitions between standing and sitting.

In some embodiments, the controller may actuate the prosthetics accommodation device to provide prolonged socket pressure release during detected prosthetic user sitting and/or resting after walking when the customization input indicates an activity volume profile where the prosthetic user loses fluid volume during walking and gains fluid volume during sitting. The prolonged socket pressure release may range between 3 minutes to 16 hours. The controller may provide vacuum assist during detected prosthetic user walking when the customization input indicates an activity volume profile where the prosthetic user loses fluid volume during walking and gains fluid volume during stand-sit/sit-stand transitions. In some embodiments the controller may be customized to provide socket immediate and prolonged pressure release during detected prosthetic user sitting and/or resting after walking when the customization input indicates an activity volume profile where the prosthetic user gains fluid volume during walking, loses fluid volume during stand-sit/sit-stand transitions, and gains fluid volume during sitting. The immediate pressure release may occur within 2 seconds to within 20 seconds of detection of prosthetic user sitting and/or resting after walking. The prolonged pressure release may range between 3 minutes to 16 hours. The controller may also be customized to provide prosthetic user alerts to reduce prolonged prosthetic user standing when the customization input indicates an activity volume profile where the prosthetic user gains fluid volume during walking, but experiences little volume change during resting (e.g., less than 1% volume change during resting). In some embodiments, alerts are provided when the system detects that a prosthetic user stands for more than 5 minutes. In some embodiments, alerts may be provided more frequently (e.g., when the prosthetic user stands for a time ranging from more than 1 minute to more than 5 minutes).

In some embodiments the prosthetics user activity is detected by a single 3-axis accelerometer. The prosthetics user activity of user donning and doffing the prosthetic may be detected by a socket proximity sensor. The socket proximity sensor may include one or more infrared sensors or ultrasonic distance sensors.

The controller may be further customized to automatically adjust the prosthetics accommodation device based on a position of the residual limb within the prosthetic socket. The controller may compare an actual high position and an actual low position of the residual limb within the prosthetic socket to an upper target and a lower target in order to adjust the accommodation device. The controller may increase fit with the accommodation device when the actual high position>upper target>actual low position>lower target. The controller may decrease pressure with the accommodation device when the upper target>actual high position>lower target>actual low position. Optionally, the position of the residual limb in the prosthetic socket may be indicated by signals from a piezoresistive force sensor.

The controller may be configured to operatively couple with a user input device. The user input device may be configured to send a signal to the controller indicating that the prosthetics user intends to stand and/or walk from a sitting position. The controller may be configured to adjust the prosthetics accommodation device in anticipation for the standing and/or walking of the prosthetics user.

Optionally, the prosthetics accommodation device includes one or more pressure pulse sensors. The controller may configured to be operatively coupled to the one or more pressure pulse sensors and configured to adjust a fluid volume recovery strategy or one or more stress locations of the prosthetics accommodation device in response to signals from the one or more pressure pulse sensors.

In some embodiments of the invention, a prosthetics device for a residual limb of a prosthetics user is provided. The prosthetic device may include an infrared sensor for detecting a presence of a residual limb. A controller may be operatively coupled with the infrared sensor. The controller may be configured to automatically adjust a prosthetics accommodation device in response to detected prosthetics user activity. The controller may be configured to recognize when the prosthetic user dons and doffs the prosthetic device in response to signals received from the infrared sensor.

In some embodiments of the invention, a prosthetic device for a residual limb of a prosthetic user is provided. The prosthetic device may include a controller configured to automatically adjust a prosthetic accommodation device in response to detected user activity. A padding may be configured to bear weight from the residual limb of the prosthetic user. A force sensor may be under the foam padding and operatively coupled to the controller. The force sensor may be configured to output a signal to the controller that is indicative of a displacement distance in response to weight bearing of the residual limb of the prosthetic user on the padding. The controller may be configured to adjust the prosthetic accommodation device in response to the displacement distance signal from the force sensor.

The invention will be better understood on reading the following description and examining the figures that accompany it. These figures are provided by way of illustration only and are in no way limiting on the invention.

### BRIEF DESCRIPTION OF THE DRAWINGS

FIGS. 1A-1C show an exemplary prototype of a bladder system-within-a-liner;

FIG. 2 shows an exemplary design of a bladder accommodation system according to some embodiments of the invention;

FIG. 3 illustrates three exemplary positions for bladder placement according to some embodiments of the invention:

FIG. 4 illustrates an exemplary embodiment where the bladder(s) and/or sensor(s) may all be contained within the liner 102 while other components such as the pumps/valves, signal conditioners, microcontrollers are separate and coupled with the socket according to some embodiments of the invention;

FIG. 5 illustrates an exemplary embodiment where the control system may be positioned within the socket at the bottom according to some embodiments of the invention;

FIG. 6 illustrates an exemplary embodiment where the whole system is contained within a bladder-liner according to some embodiments of the invention;

FIG. 7 shows an exemplary placement of one or more accelerometers according to some embodiments of the invention;

FIG. 8 shows exemplary accelerometer data for characterizing user activity according to some embodiments of the invention;

FIG. 9 shows exemplary inclination angles to differentiate between standing and sitting user activity according to some embodiments of the invention;

FIG. 10 illustrates an exemplary binary data tree for characterizing user activity based on received accelerometer data according to some embodiments of the invention;

FIG. 11 shows the sensitivity of classification accuracy compared to window length according to some embodiments of the invention;

FIGS. 12A-12D illustrate an exemplary socket proximity sensor according to some embodiments of the invention;

FIGS. 13A-13C illustrate an exemplary placement of one or more socket proximity sensors according to some embodiments of the invention;

FIG. 14 illustrates exemplary data from a socket proximity sensor according to some embodiments of the invention;

FIGS. 15A-15B illustrate data from pressure sensors configured to provided data on pulse pressures within the socket according to some embodiments of the invention;

FIGS. 16A-16B illustrate summarize exemplary classifications of prosthetic users for providing an accommodation and/or fluid recovery strategy according to some embodiments of the invention;

FIGS. 17A-17B illustrate an exemplary method and sample report according to some embodiments of the invention;

FIGS. 18A-18B illustrate an exemplary change in shape of a socket at the popliteal area in the coronal plane according to some embodiments of the invention;

FIG. 19 shows a schematic illustration of different controller states of an exemplary controller according to some embodiments of the invention;

FIG. 20 shows the expected bladder liquid injection response of a residual limb;

FIG. 21 shows an exemplary socket according to some embodiments of the invention;

FIG. 22 illustrates an exemplary placement of bioimpedance electrodes;

FIGS. 23A-23C show representative results from a subject; FIG. 23A shows the percentage of residual limb fluid volume change for each bladder liquid addition and removal; FIG. 23B shows a percentage of limb fluid volume change versus the percentage of socket volume change for each bladder liquid addition; FIG. 23C shows fluid transport compliance for each bladder liquid addition;

FIGS. 24A-24S show limb fluid transport compliance data from nineteen participants;

FIG. 25 illustrates an exemplary concept of fluid volume recovery;

FIG. 26 illustrates an exemplary placement of bioimpedance electrodes;

FIG. 27 shows the residual limb fluid volume change over time after doffing;

FIG. 28 shows the shapes of post-doffing response curves;

FIGS. 29A-29B summarize curve type results;

FIG. 30 illustrates an exemplary placement of bioimpedance electrodes;

FIG. 31 shows the results percent volume change over the thirty minute time period for the ON, OFF, and LINER groups;

FIG. 32 summarizes the results shown in FIG. 31;

FIG. 33 shows an average change in volume across all participants for each protocol, normalized to the 10 s stand after the third cycle;

FIG. 34 illustrates an exemplary placement of bioimpedance electrodes;

FIG. 35 shows example data for 2½ cycles and the beginning and the end of each REST, STAND, and WALK phase within the example data;

FIG. 36 illustrates the relationship between REST, SIT, and TRANSITION;

FIG. 37 illustrates four exemplary classification groups according to some embodiments of the invention;

FIGS. 38A-38B illustrate increased interstitial fluid pressure causing interstitial-to-venous fluid transport to dominate over arterial-to-interstitial fluid transport; and

FIGS. 39A-39B illustrate an exemplary socket insert including one or more embedded sensors according to some embodiments of the invention.

### DETAILED DESCRIPTION

The subject matter of embodiments of the present invention is described here with specificity, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

In some embodiments an accommodation device is provided that changes the volume and/or shape of the prosthetic socket in response to an activity of the prosthetic socket user. When a prosthetics socket user is active, the residual limb may reduce in volume. To continue to provide stability to the user, the accommodation system may reduce socket volume to maintain good coupling between the prosthetic socket and the residual limb of the user. When the person becomes inactive (rests), the accommodation device may increase the socket volume so that fluid may return to the residual limb to facilitate limb volume recovery. In some embodiments a system may be configured to manage limb fluid volume, accomplish limb-socket stability during gait, and perform diagnostic assessments on subjects while wearing their prosthesis.

As described in the background, the primary technologies developed to add material to the inside of the socket include either air or liquid-filled bladders. Bladders have been developed to mount to the inside socket wall and to extend through holes in the socket to a pumping mechanism. A series of bladders may be affixed to the inside socket wall at locations that tolerate increased weight bearing. Tubes may extend from these bladders through the socket wall and connect to a reservoir of low viscosity fluid positioned on the outside of the socket. One bladder may be positioned in the bottom of the prosthetic socket to serve as a pumping bladder. During activity, the prosthesis user contacts the pumping bladder with the distal end of their residual limb, forcing fluid into the bladders affixed to the inside socket surface. There may be mechanical valves within the tubes such that the bladders affixed to the inside socket surface bleed off fluid to the reservoir once they reach a specific maximum pressure. Thus, when a person is active and a residual limb reduces in volume, fluid may enter the bladders to reduce the socket volume and to facilitate socket fit. Once the person stops activity, the bladders may have a slow passive release valve that passively releases their contents to the reservoir, returning the socket back to its normal larger size. A disadvantage of this approach is that the socket is permanently modified (hole through socket wall) which some amputees do not want done to their prosthesis for cosmetic reasons.

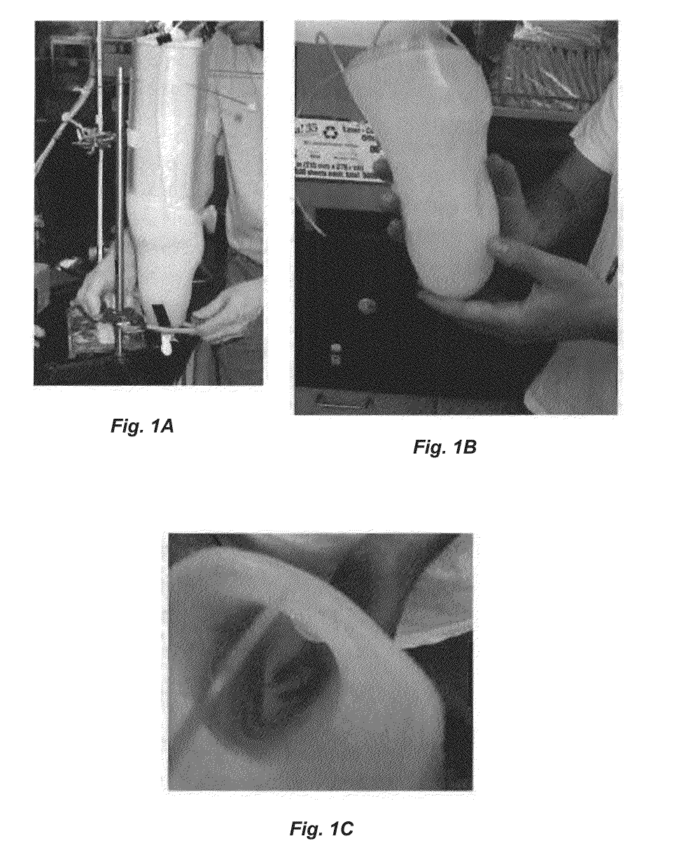

Thus, it may be advantageous to position the bladder system within a prosthetic liner instead of affixing it to the inside of the socket. It may beneficial to position the bladder system within the liner so that it may be used with existing prosthetic sockets, instead of having to make an entirely new socket for the amputee. This convenience may greatly enhance ease-of-use of the technology and the likelihood that it will be implemented. In addition, prosthetic liners are typically paid for by medical reimbursement at 9-month intervals, as opposed to sockets which are paid for at 2- or 3-year intervals. Thus, a business making liners may be potentially more lucrative than one making sockets. FIGS. 1A-1C show an exemplary prototype of a bladder system-within-a-liner. Further, a bladder liner system may be advantageous over socket accommodation devices because research has shown that liner sizes may affect residual limb fluid volume recovery (discussed further below). Accordingly, it may be advantageous to be able to directly control the volume and fit of a prosthetic liner.

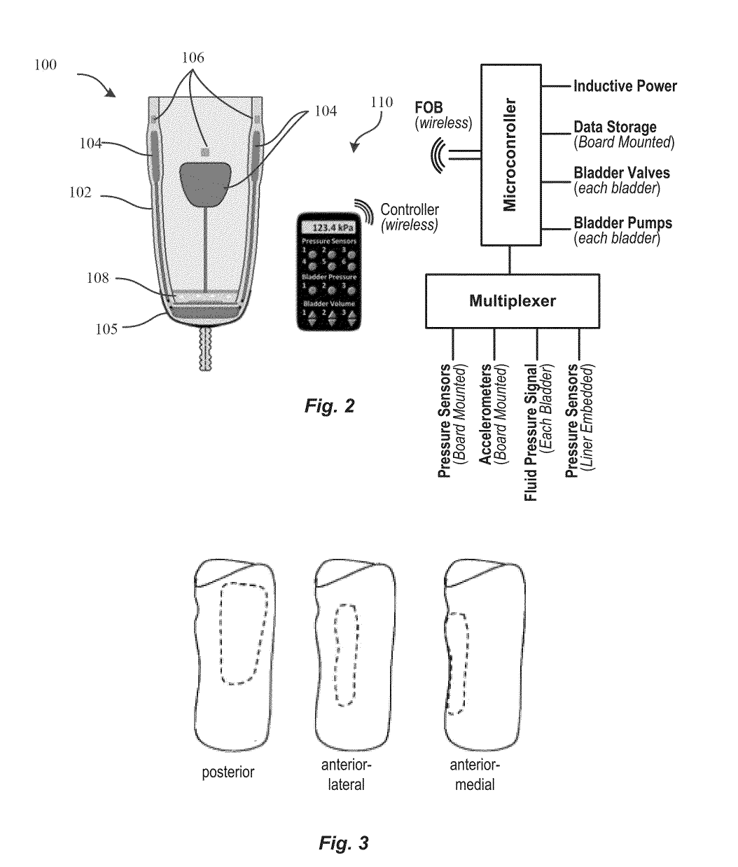

FIG. 2 shows an exemplary design 100 of a bladder accommodation system. Design 100 may include a bladder liner 102 with one or more bladders 104 operatively coupled to a fluid reservoir 105. The design 100 may further include one or more sensors 106 within the liner 102. A circuit board 108 may also be part of the design 100.

The bladder liner 102 may be an elastomeric liner. The bladder-liner systems were tested on amputee subjects and produced positive results in that the test subjects preferred the liquid in the bladders while the subjects were active. The socket was more comfortable with the bladder-liner present than not present.

In some embodiments, the bladder liner 102 may include three bladders 104. The three bladders 104 may be positioned at an anterior-lateral position, an anterior-proximal position, and a posterior position. The three exemplary positions are illustrated in FIG. 3. While some embodiments may have three bladders 104, other prosthetic users may benefit from bladder liner systems with one, two, four, or more bladders. In some embodiments, it may be beneficial to position one or more bladders at the tibial plateau (medial and/or lateral) and at the patellar tendon. These bladders may be beneficial for stabilizing prosthetics users during standing conditions. Loading the tibial plateau and/or the PTB and unloading soft tissue sites may reduce the residual limb fluid volume loss during standing. In some embodiments, volume adjustments may be placed on at the anterior and lateral tibial flares and/or in the popliteal area. If the popliteal location is placed too far distally, it may cause patient pain. Optionally, a bladder may be positioned posteriorly proximally to facilitate popliteal release during user reset periods and may enhance limb fluid volume recovery. While discussed with respect to bladder liner systems, the bladder 104 positioning discussed above may be beneficial in traditional bladder socket systems. Additionally, bladders are used in an exemplary sense, Potentially “bladder” could be replaced with any active system that changes volume—material change from a thermal or chemical input, microactuators, piezo active material, smart material etc.

The one or more sensors 106 may comprise a 3-axis accelerometer for activity/posture detection. The one or more sensors 106 may comprise socket stress sensors for assessing the need for socket size adjustment, activity/posture detection, differentiating standing and sitting. The socket stress sensors may be placed at anterior proximal, anterior distal, posterior proximal, and/or posterior distal locations. The one or more sensors 106 may include one or more limb position sensors. The limb position sensors may be used to determine activity/posture and differentiate between standing and sitting. The one or more sensors 106 may comprise bladder fluid pressure sensors. The bladder fluid pressure sensors may be used for identifying bladder fill levels and may be used as safety checks. For example, the signal may be used to determine if the degree of relief is acceptable or additional fluid removal is needed while the person is sitting. Additionally, if it is desired to relieve the posterior popliteal area because occlusion of the vasculature posterior to the tibia may be a cause of low fluid volume recovery, then the pressure measurement within that popliteal region may be used to establish when sufficient fluid removal has been accomplished. Similarly, for regions filled during relief of the popliteal area or whatever area is at risk or causes low fluid volume recovery, fluid volume/pressure may be increased until an acceptable threshold is reached.

The one or more sensors 106 may be a socket proximity sensor (SPS) for determining whether or not a lower-limb prosthesis is being worn. Details of an exemplary socket proximity sensor are described in detail below. Optionally, the one or more sensors 106 may be RFID sensors.

The circuit board 108 may include signal conditioners, a microcontroller, data storage, a power source, and/or data transmission device. The signal conditioner may be used to amplify or filter data from the one or more sensors 106. The microcontroller may be used to receive data from the one or more sensors 106 and may store the data in memory. The controller may also automatically control the accommodation system to adjust socket size based on sensor inputs and/or prosthetic user classification. For example, the controller may be configured to control fluid volume for each individual bladder collectively or independently using one or more bladder valves or pumps. In some embodiments it may be beneficial to control the fluid volume of each bladder individually. In some embodiments, a volume may be set for each bladder for a person so that there does not need to be much change in the setting from day to day. There may be a preferred “active” setting for liquid in each of the different bladders. Optionally, a “binary” mode may be used where fluid is either in the bladders or not in the bladders. By having only two settings, the person can differentiate easily and become familiar with those two configurations, easily moving back and forth between the two. It is possible, however, that over the course of the day the residual limb reduces in volume and another configuration needs to be introduced. A wireless communication to the user via smartphone for example may be integrated into the system so that the user does not need to take overlying clothes off to adjust the prosthesis. Further control methods and systems are described further below.

The data storage may store state changes, activity logs, and sensor data for later diagnosis and socket adjustment. Optionally, the data transmission device may be a wireless communication device. A wireless communication device may allow the controller to communicate with the one or more sensors. In some embodiments, the one or more sensors are RFID sensors. Optionally, the whole system may be RFID based. In some embodiments, the controller may be located on a socket while sensors are located within a liner. Wireless communication may facilitate communication between the controller and the sensors such that the controller and the one or more sensors are operatively coupled. Optionally, the accommodation device controller may be wirelessly controlled by a FOB 110.

The FOB 110 may allow user adjustments to state selections or to fluid in bladders. In some embodiments, the FBO 110 allows adjustment of each bladder's fluid content and/or pressure independently. The practitioner or the patient may set hi and low levels so that the user remains within a window of operation for each type of activity condition. It may be desirable to remain within a window of operation because too high a fluid level may cause tissue damage to the residual limb. Too low a level may be ineffective towards stability and fluid volume recovery. Optionally, in some embodiments, a user can run the system automatically. In auto mode, adjustments are made to the fluid levels and/or pressure within each bladder based off of the pressure, force, and accelerometer signals. Those signals can be used to distinguish the type of activity the person is doing (walk, stand, sit, transition, etc.) as well as the condition of the prosthesis. For example, the pressure sensors on the anterior and posterior surfaces can be used to establish the prosthetic alignment error of the prosthesis, and fluid levels can be adjusted accordingly. Control algorithms, strategies and methods are discussed further below.

In some embodiments, the system includes a battery for powering the one or more electronic devices. In some embodiments wireless recharging (e.g., inductive charging) may be used to recharge the device without requiring removal. The design may further include one or more pumps/valves to drive fluid into and/or out of the one or more bladders 104. In some embodiments the pump/valves are configured to displace the fluid either proximally or distally within a socket or a liner. A reservoir 105 may be provided that holds the liquid that goes in and out of the bladders 104. In some embodiments, reservoir 105 may be placed above circuit board 108. In such an embodiment, the distal limb of the user may act as a pump on the fluid when the person walks and may thus help drive fluid into the bladders. A shuttle lock at the distal end of the prosthesis might also be used as a fluid port to an externally-positioned reservoir, beneath the socket. That unit could be part of the distal shuttle lock system. The valves there can be adjusted accordingly.

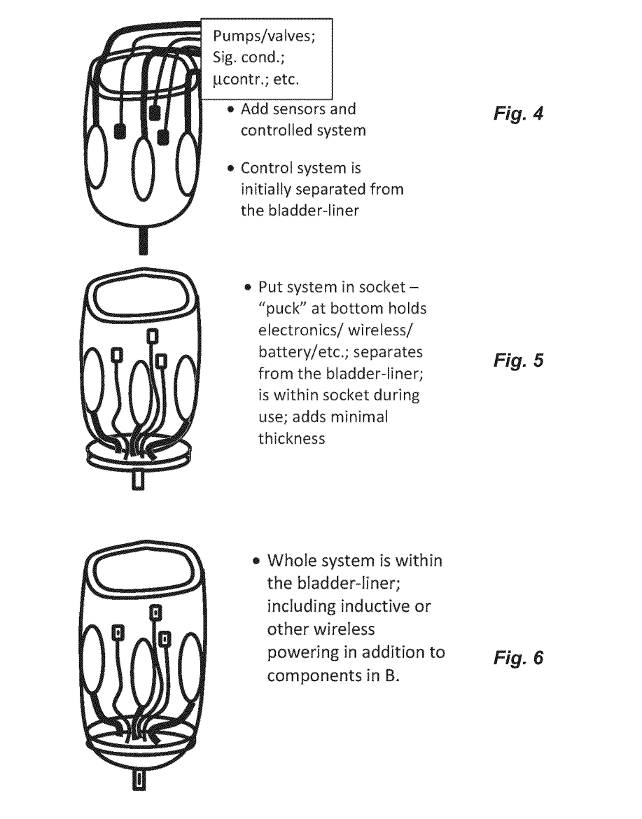

FIG. 4, illustrates an exemplary embodiment where the bladder(s) and/or sensor(s) may all be contained within the liner 102 while other components such as the pumps/valves, signal conditioners, microcontrollers are separate and coupled with the socket for example. FIG. 5 illustrates an exemplary embodiment where the control system may be positioned within the socket at the bottom. This may add minimal thickness. FIG. 6 illustrates an exemplary embodiment where the whole system is contained within the bladder-liner 102.

As discussed above, the one or more sensors 106 may include one or more three-axis accelerometers for identifying a user's activity. Prosthetists, physicians, and prosthetics researchers are challenged to describe how persons with limb loss use their prostheses outside the clinic or laboratory. Information on what prosthesis users do in their daily lives can be difficult to acquire and acquiring this information may be complicated by the range of situations and environments users encounter. The characterization of prosthesis use could be partially achieved by quantifying prosthetic wear (e.g., donning and doffing) and users' engagement in locomotor activities (e.g., walking and stair climbing) and fundamental body postures (e.g., standing or sitting). Accurate knowledge of prosthetic use in free-living conditions may enhance prosthetic prescriptions, fitting processes, and measurement of outcomes. For example, accurate knowledge of how much a prosthetic user sits or stands could be useful in determining changes in socket fit throughout the day.

While some studies have demonstrated potential for activity and posture classification based on data from body-mounted sensors, there remain challenges to clinical use such as need for multiple sensors, subject donning requirements, low storage capacities, and short battery lives. Currently available sensors may be also often restricted to short-term applications and/or require adherence to specific user protocols. Accordingly, more user-friendly and clinically-relevant solutions are needed to overcome these challenges. Thus in some embodiments an accelerometer and custom signal processing algorithm is used to identify when prostheses are being worn and to classify periods of use as movement, standing, or sitting. Use of a single sensor mounted to a prosthesis may eliminate the need for the user to attach and remove the sensor, improve wear compliance, and reduce cost.

Data from a single prosthesis-mounted accelerometer could be used to identify when a prosthetic user was wearing their prosthesis and if they were sitting, standing, or actively moving. An algorithm was designed to identify when the prosthesis was being worn and to classify actions as movement (i.e., regular leg motion like walking or stair climbing, transitioning from one posture to another, or donning or doffing the prosthesis), standing, or sitting.

The accelerometer (e.g., ActiLife ActiGraph (Pensacola, Fla.) GT3X+accelerometers) may be positioned proximal to the prosthetics user's foot. This position may ensure that the sensor is subject to higher accelerations during leg motions.

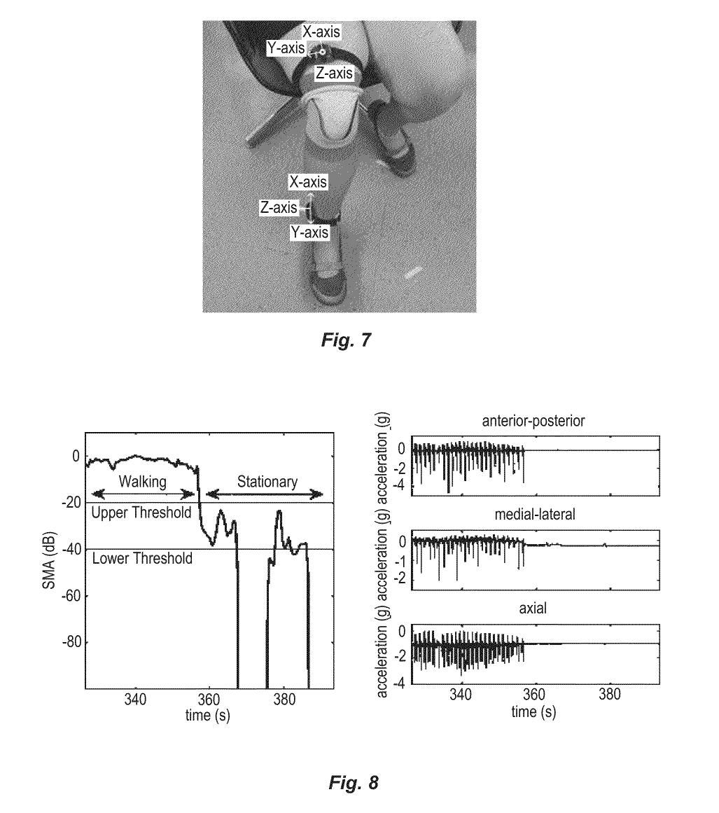

Optionally, the sensor may be oriented with the positive x-axis along the limb axis and the positive z-axis in the medial-lateral direction as shown in FIG. 7. A second accelerometer may be affixed to the anterior thigh on the same leg as the prosthesis. It may oriented with the positive x-axis along the limb axis and the positive y-axis facing to the subject's right. These locations may ensure that different postures could be differentiated using anterior-posterior acceleration data. Sampling rates of the accelerometers may be approximately 40 Hz. While higher sampling rates may be used, a 40 Hz rate may maximize a duration of data collection and still allow for the identification of relevant gait events. Further, some embodiments may use a single three-axis accelerometer to classify the prostheses use. A second accelerometer may be beneficial to validate classifications derived by the single accelerometer algorithm.

The one or more accelerometers may obtain raw acceleration data which may be post processed using custom algorithms. FIG. 8 shows an example of the acceleration data obtained by an accelerometer. The data on the right shows the acceleration data for in the anterior-posterior direction, the medial-lateral direction, and the axial direction. The data on the left shows a plot of signal magnitude area (SMA) for different activities and postures. An upper SMA threshold and a lower SMA threshold were defined to differentiate user movement from stationary activities. If the SMA was higher than the upper threshold, then the subject was considered engaged in movement. If the SMA was between the thresholds, the subject was considered stationary. If the SMA was below the lower threshold for over 320 seconds, then the prosthesis was considered doffed. The SMA plot in FIG. 8 shows SMA in dB to accurately show the difference between the thresholds. The lower threshold, set to 0.01 g corresponds to −40 dB and the upper threshold of 0.1 g corresponds to −20 dB.

A binary decision treat (BDT) algorithm may be designed to classify the windowed data. In some embodiments, data from all three axes of the pylon-mounted accelerometer may be used to determine if a user is active, stationary (i.e., sitting or standing), or had doffed the prosthesis. In an exemplary BDT, signal magnitude area (SMA) is used to determine if the prosthesis was moving or stationary within a window. SMA may be calculated by subtracting the mean from each of the accelerometer axes, integrating the absolute value of the result over a full window, and dividing by the window size. SMA may be evaluated using the following equation:

$$SMA = \frac{1}{t} {\int }_{0}^{t} \vert X ( t ) - {μ}_{X} \vert + \vert Y ( t ) - {μ}_{Y} \vert + \vert Z ( t ) - {μ}_{Z} \vert d t \qquad ( 1 )$$

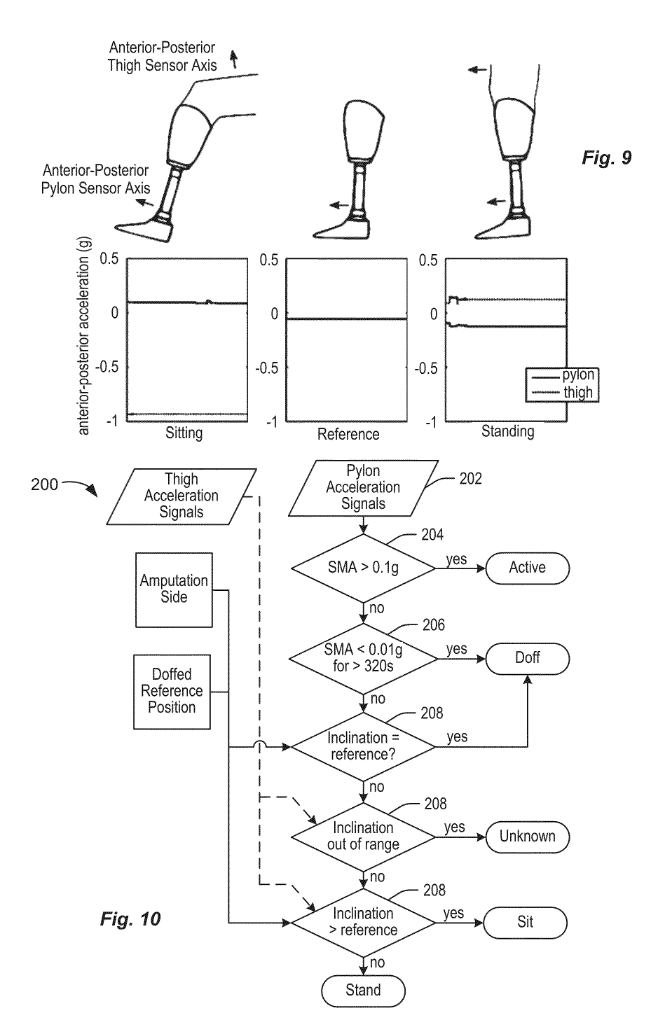

Optionally, the developed algorithm may be calibrated per the accelerometers' locations (i.e., left or right leg) in order to orient the pylon-mounted accelerometer's anterior-posterior axis. The algorithm may be further calibrated using the pylon accelerometer's inclination while the prosthesis was doffed and standing upright with the foot on the floor. The doffed position may serve as a reference to differentiate sitting and standing postures. This strategy may be effective because the anterior-posterior inclination angle (with respect to the vertical axis) was found to be greater than the doffed reference angle for sitting and less than it for standing as illustrated in FIG. 9.

An exemplary BDT 200 is shown in FIG. 10. At step 202 pylon acceleration signals are acquired. At decision 204, SMA is calculated and compared to the upper threshold (e.g., 0.1 g). If the SMA is greater than the upper threshold, the BDT 200 determines that the user is active. If the SMA is less than the upper threshold, at decision 206, the SMA is compared to the lower threshold (e.g., 0.01 g). When SMA is below the lower threshold, the subject may be deemed either to be stationary or to have doffed their prosthesis. When SMA remained below the lower threshold for more than 320 s, the prosthesis may be considered doffed. Otherwise, the prosthesis may be assumed to be donned and windows may be classified as a stationary posture (i.e., standing or sitting) depending on inclination data at decision 208. When SMA was between the lower and upper thresholds, the accelerometer data from that window may be averaged to find the inclination. Inclination may then be compared to the subject's reference inclination to determine if the subject is sitting, standing, or doffed. If the prosthesis is oriented in a way that did not correspond to one of those postures, indicated by the inclination being outside of a range that could be obtained by a sitting or standing individual, the window is classified as unknown.

When data from only the pylon-mounted accelerometer is used for classification, sitting or standing may be calculated based on the inclination of the prosthesis as determined by anterior-posterior accelerometer measurements. When data from both the pylon-mounted accelerometer and the thigh-mounted accelerometer is used for classification, the pylon-mounted accelerometer data may be used to determine movement, and data from the anterior-posterior axis of the thigh-mounted accelerometer may be used to determine posture.

Two activity thresholds were used to guide classifications. The lower and upper activity thresholds were experimentally determined via a sensitivity analysis using the laboratory-based experiment data. These thresholds were set to 0.01 g and 0.1 g, respectively While 0.01 g and 0.1 g were are used in exemplary BDT 200, other values may be used. For example the lower threshold may be a value between 0.001-0.02 g. In some embodiments using pylon and thigh data, a lower threshold of 0.008 g may be used. The upper threshold may be a value between 0.01-0.2 g. Additionally, while 320 s was used as a threshold for doffing in the exemplary BDT 200, other durations may be used. For example . . . 280-380 s.

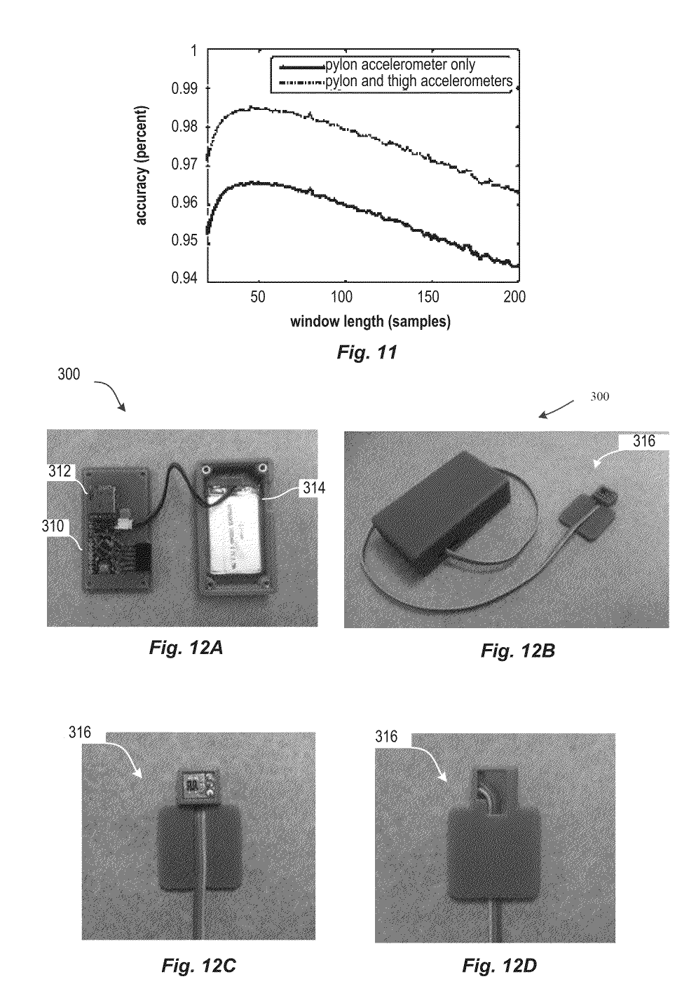

FIG. 11 shows the sensitivity of classification accuracy compared to window length. Sample window ranges may range between 20-80 samples. A window size of 40-50 (e.g., 45) samples may be used. A window size of ˜45 samples may provide maximum classification accuracy.

This activity monitoring method and system may therefore avoid the need for multiple monitors as used in other systems. The exemplary system meets or exceeds classification accuracies reported in related studies on able-bodied and elderly populations. Further, the classification system may be designed to detect sitting and standing postures as well as movement, unlike currently available prosthetic monitors such as the StepWatch3 (Orthocare Innovations, Mountlake Terrace, Wash.) and the Patient Activity Monitor (Reykjavik, Iceland).

In some embodiments the one or more sensors 106 may be a socket proximity sensor (SPS) for determining whether or not a lower limb prosthesis is being worn. An SPS may supplement 3-axis accelerometer sensor data in some embodiments to further enhance user activity classification. The SPS may be retrofitted to a socket, suspension system, and/or liner and be used to accurately represent the don/doff state of the subject in real-world conditions.

FIGS. 12A-12D illustrate an exemplary socket proximity sensor 300. The exemplary sock proximity sensor 300 includes a microcontroller 310, an SD logger 312, battery 314, and an IR sensor 316.

The SPS sensor signal is sampled by a low profile microcontroller 310 mounted to the outside of the individual's socket, and stored to an SD card 312. Additionally, in some embodiments, the signal can be wirelessly transmitted to a cellular phone or computer, for real-time analysis by a researcher or practitioner. The system may be powered by a 1000 mAh Li-Poly battery 314 which may allow it to sample continuously for up to 24 hours. This total sampling time can be increased or decreased using power management strategies implemented on the microcontroller 310. The sampling rate is also variable, from 1 Hz up to 80 Hz.

The IR sensor 316 is a low profile infrared (IR) emitter and receiver combination and is configured to be installed on or inside the socket facing inward. The SPS is able to sense an object's proximity to it by measuring the intensity of the reflected infrared light. When a subject inserts their residual limb into the socket, the IR light from the emitter is reflected off the limb, and picked up by the receiver. Higher intensity values sampled by the receiver indicate that the residual limb is inside the socket, and therefore that socket is donned. The SPS sensor is sensitive to objects within 1.0 cm, but this distance can be tuned to be longer or shorter depending on the individual application. One or more sensors can be used, with additional sensor placements increasing the accuracy of the overall system.

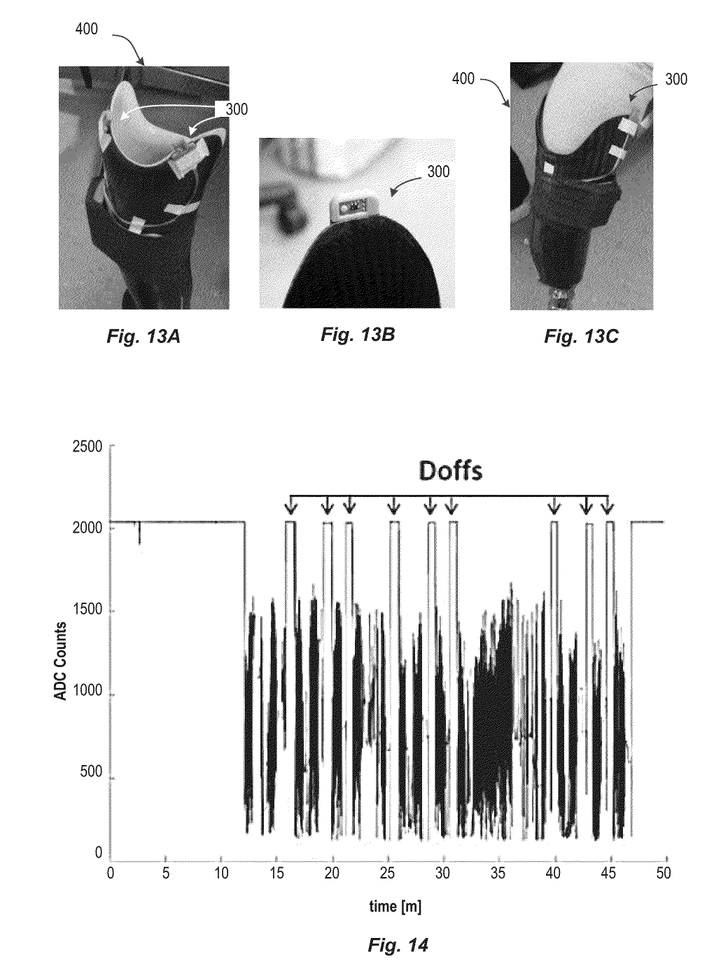

Socket Brim—FIGS. 13A-13C illustrate an exemplary socket system 400 where socket proximity sensors 300 are placed around the top of the socket brim. This may be accomplished using custom fixture hardware to conform to the individual socket. Two sensors 300 may be used, placed on the medial and lateral trimlines of the socket. This multi-sensor placement may allow good sensitivity to different postures (e.g., sitting, standing, walking) that can cause the residual limb to move around within the socket.

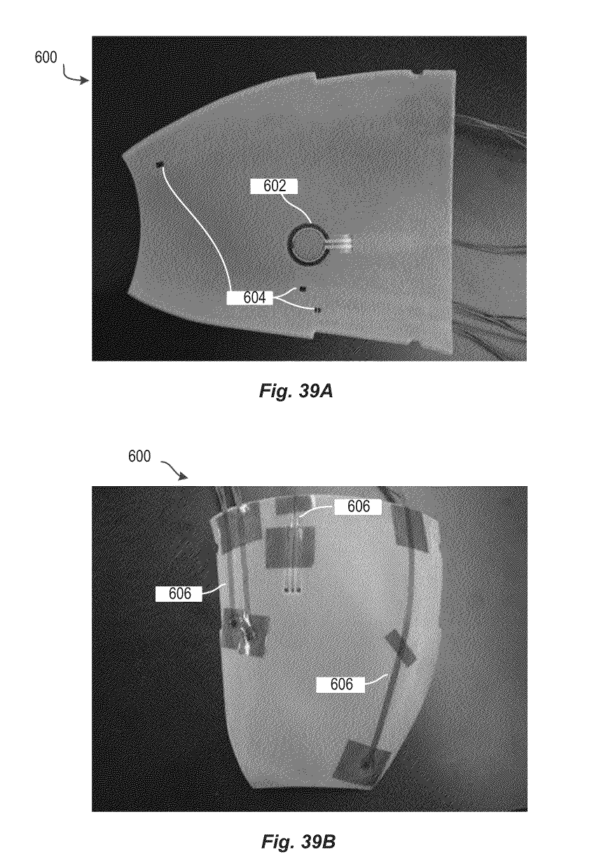

Socket Insert—Additionally, or alternatively, an exemplary socket system may be configured where the one or more socket proximity sensors are embedded within a custom 3D printed insert inside the socket. FIGS. 39A-39B illustrate an exemplary insert 600 with sensors embedded therein. The inside surface of the exemplary insert 600 is shown in FIG. 39A. The outer surface of the exemplary insert is 600 shown in FIG. 39B. As can be seen in FIG. 39A, the exemplary insert 600 may include an embedded piezoresistive force sensing resistor 602 and one or more proximity sensors 604 positioned within a part of a socket insert 600. The sensing surface of the one or more sensors may protrude through troughs or holes made on the inside insert surface so that they may be flush with the inside surface of the insert 600. FIG. 39B shows the sensor wires 606 within channels made in the outside wall of the exemplary liner 600. Wires and sensors may be affixed with boding material or tabbed in place as shown in FIG. 39B. Optionally, the socket insert may be fabricated via additive manufacturing or 3D printing methods. The proximity sensors 604 may be configured so that the sensing elements are flush with the surface and wires 606 to them run through channels or grooves made in the insert. Wires may exit out the top or bottom of the insert and connect to electronics mounted on the prosthesis. Electronic parts such as amplifiers and signal conditioners may be placed within spaces made in the insert 600. Wireless communication technology may be placed in spaces within the insert 600 so that the sensors 602, 604 can be powered and communicate wirelessly with devices mounted on the prosthesis or elsewhere. In addition to proximity sensors 604, other sensors such as force sensors, color detection sensors, EMG sensors, sweat detection sensors, temperature sensors, and other related devices may be placed in a similar manner in the insert 600. A liquid-filled bladder may also be positioned so that it sits within a trough or hole in the insert wall and is exposed to either the inside or outside of the socket. This may involve placing any number of sensors within the socket in custom locations based on the individual subject's anatomical and behavioral characteristics. Sensors 300, 604 can be placed toward the distal end of the insert to determine if a subject has not fully doffed, but only partially doffs their socket during periods of inactivity. Multiple sensors 300, 604 placed in a line from proximal to distal can be used to encode whether a socket is being donned or doffed, and the speed at which that occurs with a high degree of accuracy.

Data processing—following installation of the SPS, the system may be calibrated by having the user perform an activity (e.g., sitting, standing, walking) following a set protocol. This may be used to establish a ground truth for how the SPS data will look during donning and doffing for different postures. The subject can then leave the lab with the low-profile SPS system installed. Following their return to the laboratory, the data may be downloaded and compared against the ground-truth to determine the frequency and duration of subject doffing. A sample dataset is shown in FIG. 14. As can be seen in FIG. 14, periods of doffing are clearly distinguishable from periods of prosthetic donning Identification of doffing periods may be important for implementing automated accommodation systems and residual limb fluid volume recovery strategies as will be discussed in more detail below.

In some embodiments the one or more sensors 106 may comprise a sensor configured for detecting and/or controlling pistoning of the distal limb during user activity. The one or more pistoning sensors may be configured to measure a position of the residual limb within the socket. In some embodiments the one or more pistoning sensors may be configured to measure a distal end bearing force, a shear/pressure quotient, and/or an a/p pressure ratio. For example, in some embodiments, a pressure sensor may be located near the distal end of the limb to measure distal end bearing. Distal end bearing pressure may be measured to indicate if the person has sunk too deeply in the socket and thus that fluid should be added to the bladders to bring the person up higher in the socket. In some embodiments, a soft cushioning silicone layer may be positioned above the pressure sensor that allows some range of displacement for the residual limb. Thus a pressure sensor reading may be related to position (not just force). For running activity, a relatively low range of displacement may be desired over each step since it may be desirable to well-couple the limb to the skeleton during running. For walking, the position and the range of displacement may be higher. These ranges may be specified by the practitioner based on experience treating the patient. Further control algorithms and methods are described below. A pressure sensor such as a piezoresistive force sensor may be used for such measurements.

In some embodiments the one or more sensors 106 may be a force sensitive resistor (FSR) configured to discern the prosthetics user's intended movement (e.g., the act of standing or sitting). FSRs may be placed on both the anterior and posterior surfaces. Sitting down may cause a high pressure posteriorly and anterior distally followed by little to no pressure on the anterior surface. FSR sensors may be applied to the socket wall since the sensors may benefit from a relatively stiff backing material. While FSR measurements may suffer from signal reduction during continuous loading, such signal reduction may be overcome by calibration techniques. For example, the FSR response may be characterized in a compression testing system and then used to determine calibration correction values in a post-processing algorithm. Similar strategies may be used to correct for drift, hysteresis, and other errors. Other sensor systems and algorithms may be included that identify other activities such as walking uphill/upstairs, downhill/downstairs, turning, lying down, jumping, etc.

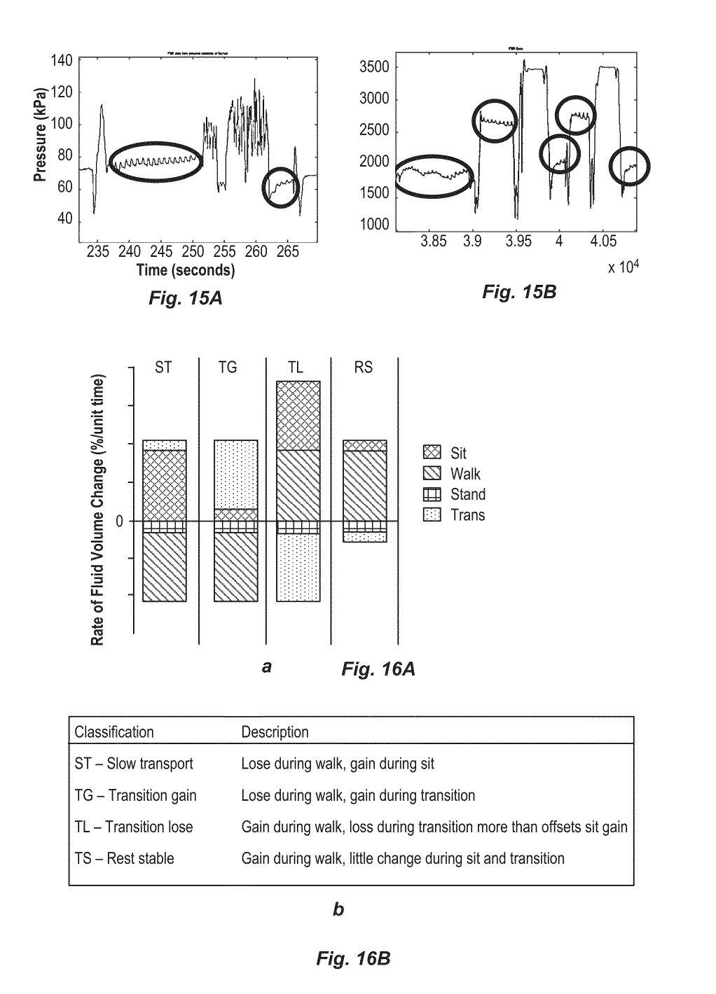

In some embodiments, a socket may be used as a diagnostic system. For example, a study was conducted by putting an FSR transducer on the socket. The amputee subject sat down, stood, etc. and a pressure pulse was measured when some subjects sat down and always when they lifted their leg while sitting down. FIGS. 15A-15B show the data from a force sensing resistor showing regions where the amputee subject's pulse was measured. Regions are circled and the subject was sitting during these times. Accordingly, positioning sensors within the liner or socket in a prosthesis that allows shape adjustment (either through modifying the bladder-liner or through modifying the socket shape) may allow the socket to be used as a diagnostic system to establish the amputee's health status. As shown in the data in FIGS. 15A-15B, the FSRs can be used to track pressure pulse. The strength of the pulse may be useful towards determining the strength of arterial drive in the amputee. The strength of the arterial drive may be important towards assessing the amputee's fluid volume recovery capability. Thus if a weak pulse is detected then the profile for controlling socket shape may be different than that for a strong pulse. For example, the amount of socket release may need to be greater for weaker pulses. By using multiple FSRs on the socket, stresses may be applied appropriately at specified locations without detrimentally affecting the person's fluid volume or fluid volume recovery. It may be beneficial to apply stress at locations where the magnitude of the pulse is not severely adversely affected by greater pressure. Accordingly, the popliteal area may benefit from relief. An accommodation device may adjust socket/liner shape during activity to counter PT loading. It may then relax as soon as the person sits and rests. Because the popliteal area of the socket may be rigid, it may be beneficial to incorporate a shell that gets pushed in or out.

The one or more sensors 106 may comprise a strain-gage plethysmography device. The device may measure changes in length as being indicative of volume change. When configured in a continuous ring, the device can be applied on the inside surface of the socket or within the prosthetic liner to measure limb enlargement in a coronal plane. In concept it could be used to measure limb volume. In some embodiments, a strain-gage plethysmography device may be used with a socket that has windows cut out of it is to use the instrumented band only in the material over those windows and may reduce the risk of out of plane motion. That information may be converted to a socket volume change, making an assumption about the shape of the membrane. For bladder-liners, the volume change can be calculated using the fluid level within the liner.

The one or more sensors 106 may include one or more pressure sensors within the socket adjustment apparatus. For example the bladders, can also serve a diagnostic purpose as well as being part of a control system to adjust socket shape according to the user's activity. In a diagnostic mode the socket shape may be modified and then the pressure response monitored. This method may provide an indication of the strain application/pressure change in the tissues, essentially the material properties of the tissues. By understanding how a person responds to a known load a better understand of their fluid transport capabilities can be provided, and then programming of the socket shape adjustment hardware may be performed appropriately. Individuals who have a residual limb that bounces back quickly, thus is highly elastic, may have better fluid volume recovery capabilities than one that does not recovery quickly. Additionally or alternatively, the test may provide an indication of people with much subcutaneous tissue. Thus data may be indicative of both elastic recovery as well as fat content in the tissues.

The one or more pressure sensors may also provide pressure measurements from the socket surface. Imbalance between anterior and posterior pressure measurement patterns (high anterior at weight-acceptance, low posterior at weight-acceptance, and vice versa during push off) may indicate a prosthetic alignment problem and need for adjustment. An excessive and high pressure on both surfaces may indicate a socket that is too tight.

The one or more sensors 106 may include a sweat detector to serve to indicate that a pumping-like action is needed to draw the sweat out of the socket, via for example a wicking material on the inside socket surface coupled with micro-pumps installed within the socket liner or wall that effect a change in socket shape.

The one or more sensors 106 may include triaxial force sensors inserted within the prosthetic liner may serve as a means to detect shear stresses on the residual limb. Shear stresses are, in general, unfavorable and to be avoided. A potential strategy is to identify a socket shape adjustment threshold such that shear stresses remain below a certain threshold. It may be advantageous to be as near to that threshold as possible so as to avoid applying excessive pressure to the residual limb, which will tend to drive out more fluid.

Sensors within the liner or socket wall may be configured to wirelessly communicate to a micro-controller mounted on the socket or pylon or elsewhere in the prosthesis so as to process collected data and determine a socket shape modification strategy.

Other variables that might be sensed include those possible with RFID technology. Some devices include: humidity, temperature, EMG (muscle activity), blood pressure, TCPO2, pulse, skin strain, and others. Sensors, if RFID or other wireless method, can also be placed within the socket wall. Since FSRs are sensitive to curvature and backing material, positioning sensors within the socket wall may be a good strategy.

Thought the bladder-liner is one approach towards adjusting the volume within the socket for the residual limb, there are other strategies that could be used to change socket shape that may benefit from the bladder positions and/or the sensors described above. Examples include opening up the socket to release pressures at certain locations so that stresses on limb tissues may be reduced, or pressure is applied at certain locations.

As described above, the one or more sensors 106 may be configured to identify the user activities and the resulting forces experienced on the residual limb from those activities. High or improperly located mechanical forces delivered to the skin-socket interface of lower-limb prostheses users can result in a variety of damaging conditions for the individual. Ambulatory discomfort and subsequent soft tissue damage caused by high or improperly applied forces can greatly reduce mobility and comfort. Additionally, daily volume changes in the residual limb can change the load-bearing regions within the socket, thereby resulting in an improperly fitted prosthesis. For example, after a rest period, some patients may experience increased residual limb fluid volume. The increased residual limb fluid volume may elevate the position of their residual limb in the socket. Because of these issues, the ability to accurately measure the applied force within the socket is potentially important both in the clinical fitting of the prosthesis, as well as towards the development of new adjustable-socket technologies.

Thus, in some embodiments of the present invention, an accommodation system may include a controller configured to automatically adjust a socket system in response to signals from the one or more sensors. With a bladder liner system, a controller design may be configured such that when the prosthetics user sits down after activity, liquid may exit the bladders so as to facilitate fluid volume recovery within the residual limb. Optionally, a controlled device may allow fluid within the bladder to be controlled via interface pressure measurements, accelerometer/inclinometer measurements on the prosthesis, or some other sensor measurement that detects what the person wearing the prosthesis is doing. In some embodiments, the bladder controller may be configured to function as follows: the controller may releases all or a portion of bladder liquid when the person sits after activity. Fluid may return immediately upon standing. Measurement of bladder liquid pressure may be incorporated into the controller algorithm so as to ensure that bladder filling does not happen too quickly such that the socket constricts the residual limb and causes the user pain. The bladders may be filled slowly as the residual limb reduces in volume from standing. An initial quick bladder fill may be important because otherwise the socket may be too loose upon standing and the person may fall.

Further, with a bladder liner system, the liner may be controlled wirelessly (e.g., RFID communication) between the instrumented liner and a unit on the socket. For example, when interface pressure sensors mounted to the inside socket wall detect a condition that requires a change in bladder fluid, the controller modifies liquid levels via communication back to the bladders. A telecommunication strategy (e.g. Bluetooth) may be used to accomplish communication. It is attractive to have the controller and bladder-liner separate since the amputee prosthesis user typically uncouples the liner from the prosthesis.

In some embodiments, the controller may also be useable with a vacuum assist system that is either operating on its own or is coupled with a bladder fluid liner or another system that allows for socket volume to be adjusted. The controller may detect the activity status of the user (e.g., sitting, standing, walking), then makes a decision on vacuum activation or socket shape modification. Typically, the socket will be increased in volume when the person sits so as to allow fluid volume recovery, and then decreased in volume when the person stands so as to accomplish good limb-socket coupling. The bladder may be activated during activity where a swing phase is present so as to facilitate fluid volume recovery during pressure relief. The concept of the combined vacuum-assist/socket shape adjustment system is that vacuum may be needed in some loading configurations and liner-bladder in others.

In some embodiments, a controller may be configured to automatically control the position of the limb in a socket to control pistoning. The controller may be configured receive data from one or more sensors that measure residual limb position or depth in a prosthetic socket. The one or more sensors can include one of the above describe sensors. The sensors can measure distal end bearing forces, shear/pressure quotients, and/or an a/p pressure ratios for example. Based on the received sensor data, the controller may keep the distal limb position at a substantially constant level during ambulation, (e.g., through bladder filling or vacuum pressures). For example, in bladder systems, bladder pressure measurements may be used to approximate the position of the distal limb. When the distal limb position lowers from a desired position, a controller may be configured to add liquid to one or more bladders of the accommodation system. When the distal limb position rises from the desired position, a controller may be configured to remove liquid to one or more bladders of the accommodation system. For distal limb position, the high and low points in limb translation may be measured by the distal limb position sensor. The points may be compared to the lower and upper bounds that are set by the practitioner during fitting. There may be 4 states for limb position. Ranking from high to low position:
Actual-hi, Target-hi, Actual-lo, Target-lo: fluid may be added to bladders—enlarge the socket volume to move the limb lower;
Target-hi, Actual-hi, Target-lo, Actual-lo: fluid may be removed from bladders—reduce the socket volume to move the limb higher;
Target-hi, Actual-hi, Actual-lo, Target-lo: may indicate not enough pistoning to allow effective reperfusion during swing phase; may need to change distribution of fluid volume in bladders;
Actual-hi, Target-hi, Target-lo, Actual-lo: may indicate too much pistoning and too low in socket during stance—a proximal to distal difference in bladder fluid may be beneficial after an initial increase in all bladder volumes.

The Actual-lo and Target-lo may be considered first so as to ensure the person is not too low and to make that bladder liquid volume change to get them higher, and then subsequently consider the distribution of liquid volume among the bladders.

If shear/pressure information is used then sh/pr may be lowered to a ratio that is within an acceptable range.

If interface pressure is used then multiple pressure sensors may be used as contact sensors.

As mentioned above, a controller may be configured to automatically adjust an accommodation system. In some embodiments the controller may be used to control the socket fit as the prosthetic user engages in daily activities. The controller may be configured to provide an accommodation strategy that also provides for periods of residual limb fluid volume recovery.

Whatever the technique used to adjust the space available to the prosthetic limb within the socket, a fundamental challenge is to determine what level of volume adjustment is needed, when it is needed, and where it is needed. This information dictates how a system to accommodate limb volume change should be designed.

Thus, in another aspect of the invention, systems, methods, and devices are provided for controlling and adjusting accommodation devices during prosthetic user activity. Part of the challenge towards creating an effective adjustable socket system for people with trans-tibial limb loss is that changing the bladder liquid volume may affect residual limb volume. The interplay between filling or emptying the bladders and changes in residual limb fluid volume can be important in developing an adjustable socket system because socket fit can be very sensitive to small volume changes, especially for people with trans-tibial amputation. A study on 10 subjects with trans-tibial limb loss showed that an experienced practitioner, using static fitting procedures, identified sockets oversized by just 0.25 mm mean radial thickness (approximately half the thickness of a new 3-ply Soft Sock (Knit-Rite) while worn on a residual limb during walking) as needing sock addition or other modification.

Further, surprisingly, not all prosthetics users respond to changing socket/liner volume the same way. For example, not all prosthetics users lose residual limb fluid volume during walking Additionally, not all prosthetics users gain residual limb fluid volume during resting. Thus, prosthetics users may need customized accommodation and recovery strategies. The research which led to the unexpected discoveries is discussed in detail further below. According to the research, the prosthetics user response may depend on user activity (walk, stand, sit) and the ability for users to recover residual limb volume. Thus predicting how different activities affect fluid volume change alone may be difficult. Diagnostic measurements that characterize a patient's fluid change profile may make daily volume changes more predictable and allow for customization of an accommodation device and recovery strategy. For example, healthy people may have an intact fluid volume control system and thus may continue to decrease in volume when socket volume is reduced. Sick people, more specifically those who have a poor fluid volume control system (e.g., users with peripheral arterial disease (PAD)), may not decrease in volume. Thus the use of pressure as a control variable may work better for sick people compared to healthy people with an intact fluid control system. However, because they are unhealthy, their soft tissues may be more susceptible to detrimental effects of socket pressures. Therefore, a recovery strategy may be implemented that does not involve applying the same pressure continuously so as to avoid continual socket pressures.

Thus, in some embodiments, the prosthetics user may first be classified into groups based on their residual limb fluid response during different activities. In some embodiments, the user may be classified into one of four groups summarized in FIGS. 16A-16B.

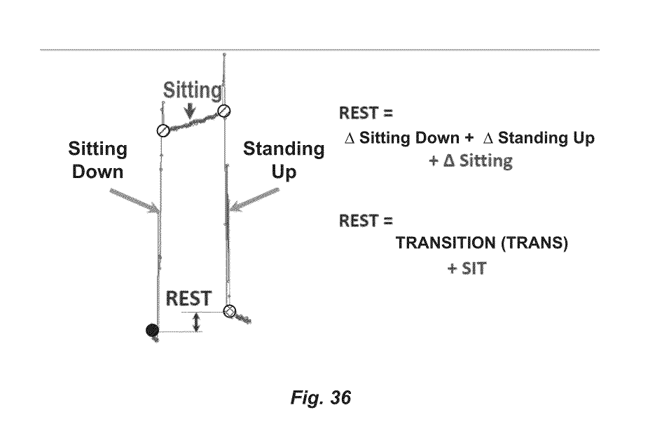

The prosthetics user may be classified as slow transport (ST) subject when they lose fluid volume in their residual limb during walking and gain fluid volume in the residual limb during sitting. The prosthetics user may be classified as a transition gain (TG) subject when they lose fluid volume in their residual limb during walking and gain during stand-sit and sit-stand transitions (“transition”). Transitions are discussed further below with FIG. 36. The prosthetics user may be classified as a transition lose (TL) subject when they gain fluid volume during walking and lose fluid volume during transition (transition loss more than offsets sitting gains). The prosthetics user may be classified as a rest stable (RS) subject if they gain volume during walking and experience little change during sit and transition.

Table 1 provides a ranking of accommodation strategies based on prosthetics user classification.

TABLE 1

| | | Socket | Sock | |
|---|---|---|---|---|
| Activity/volume | Augmented | Modification/ | Application/ | Activity |
| Classification | Suspension | Change | Removal | Modification |
| Slow transport | 4^{th} | 3^{rd} | 1^{st} | 2^{nd} |
| Transition gain | 1^{st} | 2^{nd} | 3^{rd} | 4^{th} |
| Transition lose | 3^{rd} | 4^{th} | 2^{nd} | 1^{st} |
| Rest stable | 2^{nd} | 1^{st} | 4^{th} | 3^{rd} |

Accommodation strategies may depend upon which group the patient is classified. Augmented suspension accommodation may be provided by an elevated vacuum device. Socket modification/change may be provided by the insertion of pads or other material inside the socket or sockets being replaced altogether. Sock application/removal may be provided when the patient adds socks at some point during the day and removes them at other points. Activity modification may be provided by recommending socket doffing at points during the day or some other form of changing activity. In some embodiments, a controller may be configured to send user reminders or alerts to rest and/or doff in order to modify user activities.

For users classified as ST, socket application/removal may be the most effective accommodation strategy. Optionally, activity modification may be used to provide some fluid volume recovery. For users classified as TG, augmented suspension may be the most effective accommodation strategy and socket modification/change may be considered. For prosthetic users classified as TL, activity modification may be the most effective accommodation strategy, followed by sock application/removal.

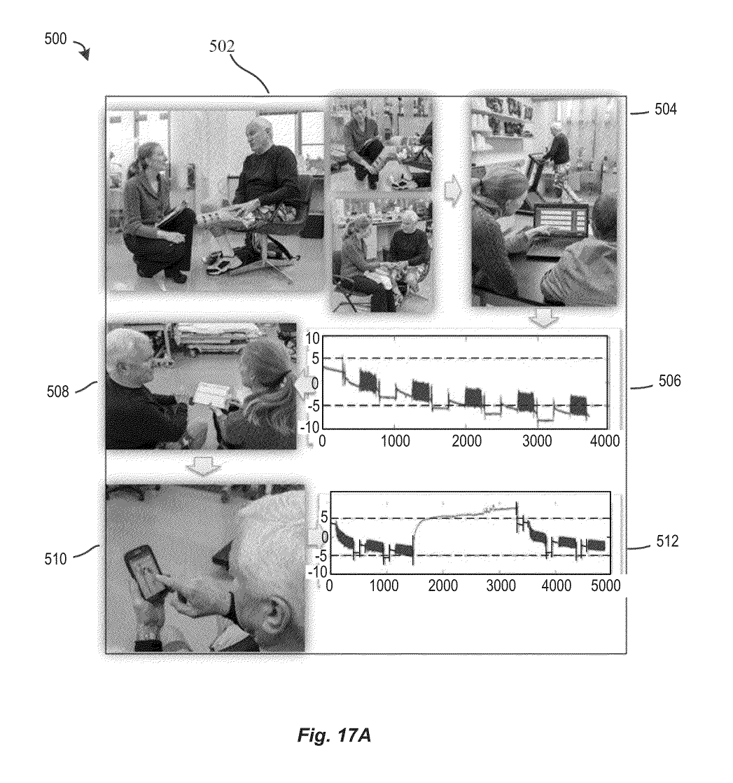

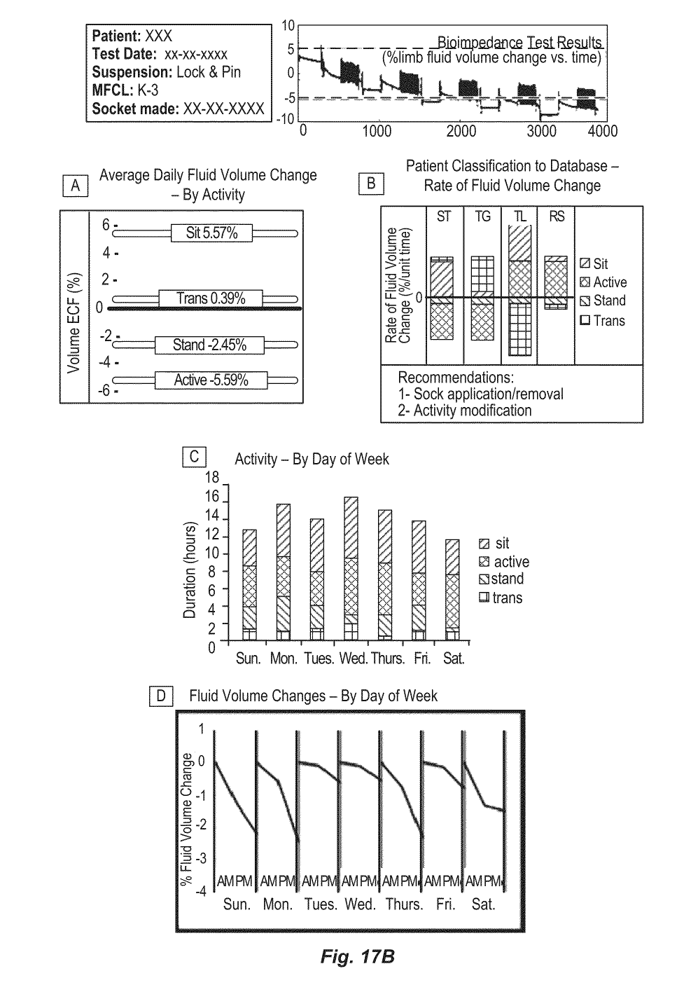

FIG. 17A shows an exemplary method 500 according to some aspects of the invention. At step 502, a practitioner may discuss socket fit and volume management issues troubling the patient, inspect the residual limb for signs of injury, and determine sock use patterns. At step 504, the practitioner may perform an in-clinic bioimpedance test to look at limb fluid volume data as the patient walks (on a treadmill for example). At step 506, the fluid volume data may be analyzed and compared to an upper and lower fluid volume limit. At step 508, the patient's activity/volume profile may be analyzed and an accommodation device controller may be configured to provide a recommended accommodation strategy based in-part on the profile. At step 510, the controller may carry out the recommended accommodation strategy, for example by sending reminders to doff a prosthesis and/or liner for a time period. At step 512, the effectiveness of the accommodation strategy may be tested to determine whether the residual limb fluid volume was maintained within an acceptable range.

FIG. 17B illustrates a sample test report. The report may include the average daily fluid volume change by activity. The report may include patient classification based on the rate of fluid volume change in response to activity. The patient classification may include recommendations for residual limb fluid recovery. The report may further include a summary of user activities over the span of a time period such as a week. Additionally, the report may include fluid volume changes by the day of the week.

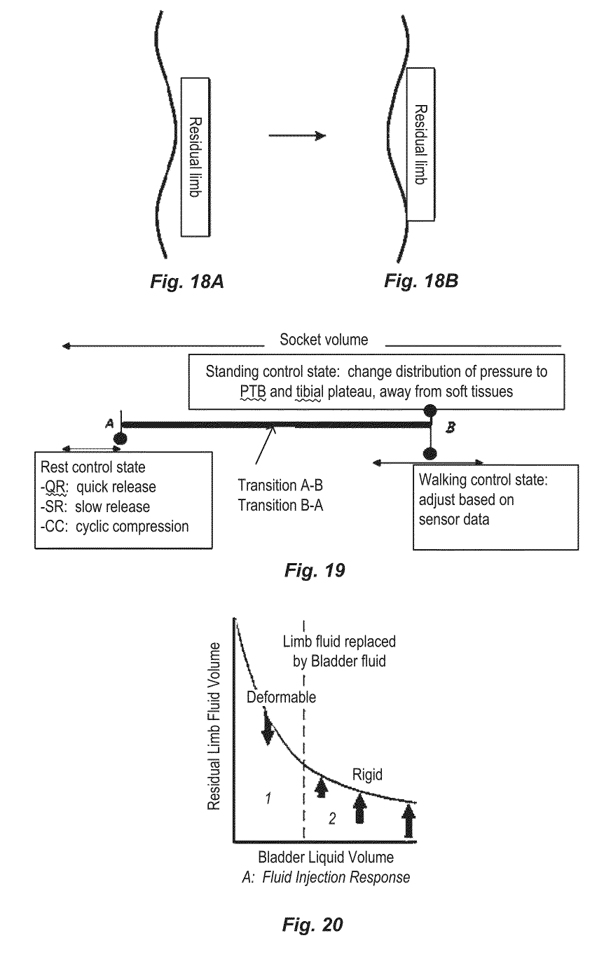

In some embodiments, an accommodation device may provide release at certain locations on the limb to accomplish fluid volume recovery rather than requiring release of the whole limb. Certain locations on the socket may be configured to provide support and may be loaded while the remainder of the socket may be unloaded to facilitate fluid recovery. In some embodiments, a socket shell may be provided that has release windows that allow the residual limb to recover volume while the remaining skeleton of the socket may provide support in certain load bearing regions. The windows may be enlarged or reduced using a flexible material on the inside surface. Unloading the posterior proximal aspect of the socket (popliteal area) may be advantageous because release of the posterior vasculature may enhance blood return to the residual limb. The hamstring tendons, or lateral and medial to them, may be locations to load since those tendons are not vascularized and may not be too strongly affected by load in terms of obstructing fluid volume recovery. The popliteal fossa, which is between the hamstring tendons may be problematic and in need of relief. Accordingly, in some embodiments, load may be applied to some locations without significantly affecting fluid volume recovery. A socket that changes shape so that the popliteal area in the coronal plane transforms from the shape shown in FIG. 18A to the shape shown in FIG. 18B may be a beneficial method for residual limb volume recovery. In some situations the posterior proximal section could be relieved.

Optionally, socket changes may be applied along a proximal-distal segment as opposed to at one coronal plane level. Changes may be applied on the anterior flare of the tibial, medial flare of the tibia, and a posterior region. The areas of adjustment may be long and thin. Applying reduction around one coronal plane may tend to occlude blood flow distal of the location of reduced circumference. This constriction may put the residual limb at risk of vascular injury. If applied along a proximal-distal path then the entire region may be pushed in or out, and the occlusion problem may be avoided.

A schematic illustration of different controller states of an exemplary controller is shown in FIG. 19. “A” represents the Base Rest socket volume setting. “B” represents the Base Active socket volume setting. The Rest control state represents the state the controller is in when sensors determine the person is resting. The Standing control state represents the state the controller is in when the sensors determine the person is standing. The Walking control state represents the state the controller is in when the sensors determine the person is walking.

There may be a Rest state, Active/walking state, and a Standing state. Transitions between states may be identified through accelerometer and/or force sensing resistor (FSR) data (An FSR is used as an example here; other force sensing devices could be used) using the one or more methods described above. A to B (and B to A) may be a relevant transition phase because of the large socket volume difference between B and A.

A and B—Points A and B may be socket volume levels set by the user or practitioner during fitting of the device. A is the Rest state volume of the socket such that the amputee is able to wear the prosthesis but it is not tight enough for activity. B is the Active state (walking or standing) volume of the socket such that the prosthesis is stable for standing or walking Based from the concept diagram of FIG. 19, B may be a setting partway down the slope of the limb volume-liquid volume curve shown in FIG. 20, such that the prosthesis is stable during activity.

Walking State—Because, some subjects gain fluid volume during walking and some subjects lose fluid volume during walking, both increases and decreases in socket volume relative to the B position may be necessary during walking Socket volume may be enlarged during walking for subjects who increase in limb volume during that time so as to facilitate fluid volume recovery and thus reduce their overall volume loss over the day. Socket volume may be decreased during walking for subjects that decrease in limb volume during that time.

Stability/pistoning may be monitored using the controller and one or more sensors. From the above possibilities, shear/pressure quotient, distal limb position, and a/p pressure ratios are possible variables to use to establish presence of pistoning. Stability may be related to pistoning. Pistoning may be related to shear/pressure quotient. Thus pistoning and shear/pressure ratio may be a consideration. It might also be appropriate to consider the relative loading in four locations—anterior proximally, anterior distally, posterior proximally, and posterior distally—since the relative loads may indicate if a lot of bending is being induced on the limb in the socket and thus that the socket is too loose and putting soft tissues at risk. It may be advantageous to have the largest socket volume possible while keeping pistoning within acceptable limits, sh/pr ratios acceptably low, and/or acceptable a/p pressure ratios.

If the residual limb reduces in volume during walking, then the socket/liner may be configured to decrease in volume with it. This may accentuate limb fluid volume loss but may ensure that the person remains stable and does not fall. Patients who lose fluid volume over the short term typically have PAD and may not be very ambulatory and may not walk long distances. Potentially, socket volume may not need to be reduced because they ambulatory periods are so short. However, these users may benefit from socket/liner volume increase and for a long period after they stop walking.

The exemplary controller may be configured to prevent socket volume to be reduced beyond a minimum level, which may be specified by the practitioner. The controller may not necessarily seek to accomplish that level, but instead it serves as a minimum set value so that the limb is not traumatized by overstressing. In some embodiments, a bladder pressure measurement and a bladder pressure threshold may be used to signal a minimum socket/liner volume.

Standing—All people wearing prosthetic limbs showed limb fluid volume loss during standing. Those losses may be reduced by loading only at specific locations on the socket, away from soft tissue sites. The PT and tibial plateaus (a kneeling type action) are likely good locations to tolerate load. Thus during long standing, the socket should become smaller at those locations and possibly others.

Rest—Rest may be a phase for which the control strategy varies between prosthetics users, depending not just on gait and gait history but also on the health and qualities of the person. The practitioner may configure the controller to execute the appropriate modality depending upon results from clinical exam of the amputee.

Quick release—This may be a strategy beneficial for people who lose a lot of fluid volume during transition, in other words those that will experience less fluid volume loss right after sitting down if their socket is not constrained. Liquid from the bladders may be slowly released as soon as the person sits down and then accentuate that release once they are still. Optionally, volume release may be provided before the person stops walking and sits down so as to initiate fluid volume return.

Slow release—This mode may be used on people who tend to easily become edematous, people with weak arterial drive who may not benefit by quick release and may not be very stable and thus at risk of falling if quick socket release is implemented. The strategy here may be to either have socket release happen slowly after a set time after sitting, instead have a gradual slow socket release initiated upon sitting, and/or to use the popliteal pressure pulse to decide the magnitude of socket release.

Cyclic compression—This mode may be used on people who become edematous as a result of socket release, and may benefit from having their limb volume reduced so as to be able to re-don their prosthesis. Cyclic compression may be applied in a manner consistent with compression therapies discussed in the edema treatment literature, which specify intervals and modes of compression application. Monitoring may be performed during sitting to see if adequate relief has been accomplished and thus that cyclic compression should be terminated.

Rest-to-Stand and Stand-to-Rest Transitions—Depending on how much recovery was accomplished during Rest, the return of socket size might not need to be to the original socket size but may instead be an intermediate value. The socket size may be determined indirectly through the pressure in the bladders, but more accurately by the resistance seen by the fluid driving system (assuming a fluid bladder system is used). The motors may need to be low torque so that excessive compression of the residual limb does not occur. This algorithm may reduce the socket size to a point of a certain resistance and then subsequent socket volume decrease may occur slowly over time if it is needed.

The above controller strategy may have the following variables to be set by the user/practitioner:

Inactive (Rest) state liquid levels in bladders—This initial state may be 0 cc for each bladder, but can be set differently by user/practitioner. For other socket adjustment strategies, a value may need to be specified. A larger than 0 cc value may be needed to keep the socket from falling off of the limb during resting.

Active ambulatory state liquid levels in bladders—A specific cc level may be set for each bladder (probably at least 2 bladders, maybe as many as 5 if the tibial plateau is loaded separately)—these values may be determined through a separate algorithm run on the patient.

Standing state relative liquid distributions in bladders—the distribution may be determined through a separate algorithm run on the patient.

Appropriate for Quick Release (QR)—y/n—may depend on results from clinical diagnostic test.

Appropriate for Slow Release (SR)—y/n—may depend on results from clinical diagnostic test used on a patient.

Appropriate for Cyclic Compression (CC)—y/n—may depend on results from clinical diagnostic test.

Vertical position of limb in socket for ambulation (upper and lower bound (mm))—may be determined through a separate algorithm run on the patient.

Shear/pressure range for ambulation (upper and lower bound)—may be determined through a separate algorithm run on the patient.

A/P pressure or pressure-shear ratios—may indicate excessive bending of limb which may indicate a socket that is loose.

Active ambulatory state—distal limb position, shear/pressure ratio out of the interface sensors, and/or ratio of a/p pr/sh stresses may be monitored to determine if a change in bladder liquid volume is necessary.

Each of the approaches described above for Active, Rest, and Stand may effectively reduce limb fluid volume losses in amputee test subjects.

After 10 minutes of sitting with socket pressure release via bladder liquid removal, it was discovered that during the first walk after release, limb fluid volume in the anterior and posterior regions of the limb were higher than pre-liquid-release levels.

Limb fluid volume recovery was much higher in the anterior region than the posterior region presumably because the anterior surface was completely unloaded in the socket when liquid was released from the bladders.

The controller may be configured to operate by releasing liquid volume right after sitting so as to induce limb fluid volume recovery. The controller may be configured to detect intent to sit and then release the socket in such a way that the prosthesis does not apply excessive pressures to the limb but at the same time does not fall off the limb.

The controller may operate based on a feedback from sensors within the socket to ensure excessive limb swelling or reduction is identified and managed.

One or more computing devices may be adapted to provide desired functionality by accessing software instructions rendered in a computer-readable form. When software is used, any suitable programming, scripting, or other type of language or combinations of languages may be used to implement the teachings contained herein. However, software need not be used exclusively, or at all. For example, some embodiments of the methods and systems set forth herein may also be implemented by hard-wired logic or other circuitry, including but not limited to application-specific circuits. Combinations of computer-executed software and hard-wired logic or other circuitry may be suitable as well.

Embodiments of the methods disclosed herein may be executed by one or more suitable computing devices. Such system(s) may comprise one or more computing devices adapted to perform one or more embodiments of the methods disclosed herein. As noted above, such devices may access one or more computer—readable media that embody computer-readable instructions which, when executed by at least one computer, cause the at least one computer to implement one or more embodiments of the methods of the present subject matter. Additionally or alternatively, the computing device(s) may comprise circuitry that renders the device(s) operative to implement one or more of the methods of the present subject matter.

Any suitable computer-readable medium or media may be used to implement or practice the presently-disclosed subject matter, including but not limited to, diskettes, drives, and other magnetic-based storage media, optical storage media, including disks (e.g., CD-ROMS, DVD-ROMS, variants thereof, etc.), flash, RAM, ROM, and other memory devices, and the like.

Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

The following disclosures detail research conducted that lead to some of the surprising and unexpected findings disclosed above.

### Effects of Liquid Bladder Use on Residual Limb Fluid Volume Research

To better understand how filling and emptying liquid bladders affects limb fluid volume, research was conducted on a group of subjects with trans-tibial amputation. The results of the research provides a base understanding from which a strategy may be developed to set bladder liquid volumes for individual patients. The insight facilitates the development of control systems to automatically adjust socket size for prosthesis users so as to induce a stable limp fluid volume over a period of time (e.g., a day) with low risk of injury. The preliminary research addressed the following questions:

What range of bladder liquid volume injection was acceptable?

Was there a consistent relationship between liquid volume injected and limb fluid volume change?

Did limb fluid volume return to pre-bladder-injection levels when bladder liquid was removed?

If not, as more liquid was injected did the limb become non-compliant (i.e. fluid out=fluid in for an inject/remove cycle)?

Preliminary Research Methods

Subjects—

Volunteers were considered for inclusion in this study if they had a trans-tibial limb amputation as least 18 months prior and were using a definitive prosthesis safely and comfortably for at least 5 h/day. Other inclusion criteria included the capability of treadmill walking at a self-selected walking speed for at least 2 min continuously. The residual limb needed to be at least 9 cm in length to allow adequate distance between voltage sensing electrodes (see below). Exclusion criteria included current skin breakdown, inability to wear the prosthesis for at least 1½ h continuously, and inability to shift from standing to supine posture within 30 s (necessary for vascular tests). The bladders were positioned so that the long axes of the bladders were parallel to the limb longitudinal axis. If the practitioner deemed the socket too small to allow all four bladders to be positioned, then one of the bladders was removed. Tubes from the bladders exited at the socket brim. ‘Y’-connectors were used to connect the bladder tubes to one common tube. A stopcock was positioned at the end of the common tube to allow a 100 ml syringe to be connected to add water to the bladders during the study, or to close off the system so that no liquid escaped. The amount of liquid to each individual bladder was not controlled but instead the liquid within the entire bladder system was controlled.

Sockets—

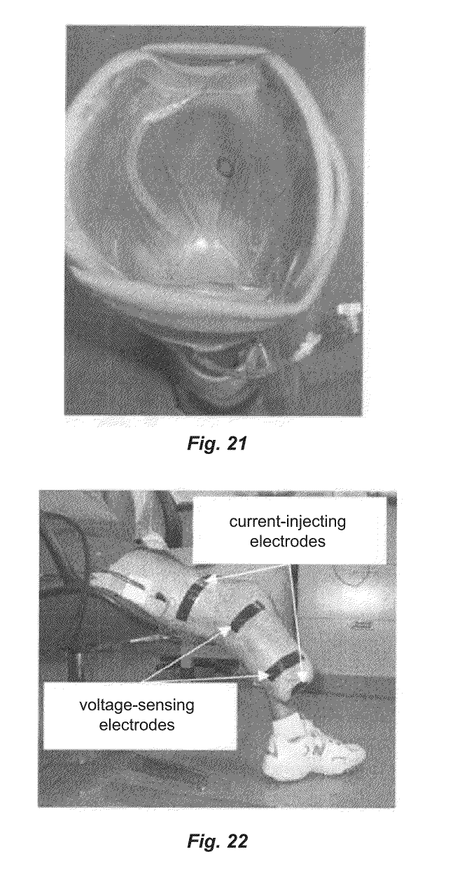

Participants used their regular prosthesis during the study. FIG. 21 shows an exemplary socket. Polyurethane bladders of external dimension 12.7 cm×3.5 cm (liquid-filled region dimension 11.8 cm×2.5 cm} and thickness 0.8 mm when unfilled were affixed to the inside socket surface with double-stick tape at the following locations: lateral tibial flare; medial tibial flare; posterior lateral, distal to the popliteal fossa; and posterior medial, distal to the popliteal fossa. The bladders were positioned at a level on the limb longitudinal axis between the voltage-sensing electrodes and in some cases at the level of the proximal voltage-sensing electrode. The locations were not expected to restrict blood flow in the residual limb. The bladders were made by radio-frequency (RF)-welding together two pieces of 15-gauge polyurethane film at the edge. A 2.5 mm inner diameter polyurethane tube was RF-welded into place at the same time to provide a channel for liquid to enter and exit the bladder. A 10.2 cm long tube made of Tygon was coupled to the end of the polyurethane tube such that it was within the bladder. Without this tube, liquid was difficult to remove from the bottom of the bladder when the subject wore the socket.

Instrumentation—

Bioimpedance analysis was used to assess residual limb fluid volume. Bioimpedance is a technique that may be used for body composition/body fat assessment, and fluid imbalance detection in hemodialysis patients. In four-electrode segmental limb bioimpedance analysis, electrical current may be applied across two outer electrodes while voltage is sensed between two inner electrodes. The applied current may be of low amplitude (50 μA to 700 μA) and may be applied over a frequency range of 5 kHz to 1 MHz. The voltage and current signals may be demodulated to determine a magnitude and phase difference and then fit to a model to determine extracellular and intracellular impedance. Intracellular and extracellular components can be distinguished because high frequency current may travel well through both intracellular and extracellular biological material while low frequency, which does not penetrate cell membranes well, may travel primary through extracellular biological material. Using a limb segment model, impedances may be converted to fluid volumes. Extracellular fluid volume may be solely considered in this analysis because of its possible strong influence on short-term residual limbfluid volume change.

In this study a commercial bioimpedance analyzer (XiTRON Hydra 4200, lmpediMed, San Diego, Calif.) was modified and used for testing residual limbs of people with trans-tibial limb loss. Briefly, custom electrodes were made using conductive tape (ARCare 8881, Adhesives Research Inc., Glen Rock, Pa.) and an underlying hydrogel (KM10B, Katecho, Des Moines, Iowa). Multi-stranded silver-plated copper wire (32 AWG, New England Wire, lisbon, New Hampshire) was used for the electrode leads. The outside of the conductive tape was covered with Tegaderm™ (Transparent Film Dressing, 3M, St. Paul, Minn.). To prevent air from escaping adjacent to the wires as they passed under the sleeve and out at the thigh which could reduce suspension, the four wires were placed on a Band-aid and covered them with Tegaderm. A custom connector with gold-plated pins (WPI Viking, Cooper Interconnect, Chelsea, Mass.) was used to connect the custom electrodes to the instrument, and the provided connector was replaced with a robust cable connector (MS3116F106S, Burndy, Manchester, N.H.) to reduce noise from mechanical motion. The peak-to-peak fluctuation in signal while the subject stood bearing weight was less than 0.1% of the limb fluid volume.

Using custom Matlab (v.7.10, MathWorks, Natick, Mass.) code implemented on a PC (Latitude D620, Dell, Round Rock, Tex.), the biompedance data was plotted in approximately real time (3 s delay) at a 0.5 Hz sampling rate. The visual data presentation during the test session helped identify any set up problems if they existed.

Protocol—

On a separate day before biompedance testing but not more than 12 month prior, the subjects were tested for presence of high blood pressure (orthostatic blood pressure (OBP)), peripheral arterial disease (ankle-brachial index (ABI)), and venous stasis (ambulatory strain-gage plethysmography (ASGP)). An electronic blood pressure measurement unit (HEM-775, Omron, Kyoto, Japan) was used for OBP testing, a commercial segmental limb pressure measurement system (TD312 Cuff Inflator, MVlO Manifold Selector, and SC12 and SClO cuffs, Hokanson, Bellevue, Wash.) was used for ABI testing, and a commercial plethysmography system was used for ASGP testing (EC6 Plethysmograph, Hokanson). Collected data were interpreted by a practicing endocrinologist using standard clinical procedures.

Subjects were requested not to consume caffeine or alcohol on the morning of the day of bioimpedance testing. After a subject arrived at the lab, he or she sat in a chair with the prosthesis donned and the foot supported on the floor for 20 min while the research practitioner queried the subject about medical history and prosthesis comfort. The entire testing protocol including audio was recorded by video camera so it could be reviewed later to capture all verbal information provided by the subject and research practitioner (laser Axon, Taser International Incorporated, Scottsdale, Ariz.). The subject's mass and height with the prosthesis donned were recorded. The subject then doffed the prosthesis, and the research practitioner inspected the residual limb for signs of injury. If injury was noted then the subject was referred to his or her regular practitioner for treatment and possible socket modification. If injury was not noted then liquid bladders were affixed to the socket with double-stick tape. The bladders were empty at the outset of the trial, but the tubes between the stopcock and bladders were primed with water Skin sites at which electrodes were to be placed were cleaned with sandpaper (Red Dot Trace Prep 2236, 3M, St. Paul, Minn.). A thin layer of ultrasonic coupling gel (Couplant D, GE Panametrics, West Chester, Ohio) was applied between the hydrogel and subject's skin, and then applied the electrodes. FIG. 22 illustrates an exemplary placement of bioimpedance electrodes. The electrodes were applied relative to anatomical landmarks on the limb. The proximal voltage-sensing electrode was positioned at the level of the patellar tendon on the lateral posterior limb surface. The distal current-injecting electrode was positioned as far distally as possible but still on the relatively cylindrical portion of the residual limb. The distal voltage-sensing electrode was placed at least 3.0 cm proximal to the distal current-injecting electrode. The proximal current-injecting electrode was placed on the thigh above the knee but under the elastomeric liner or suspension sleeve. Limb circumference was measured at the levels of the voltage sensing electrodes and the distance between voltage sensing electrode centers was recorded.

The subject was asked to don the prosthesis and then sit for 90 s with the prosthesis supported by the floor. Care was taken to ensure the subject maintained a good sitting posture. Too much knee flexion may occlude blood flow, and too much extension may cause a slouching posture. The subject then stood for 90 s on a support platform with an electronic weight scale (349KLX Health-O-Meter, Pelstar, Alsip, Ill.) embedded in the surface. Then the subject walked on a treadmill (Quinton Clubtrack, Cardiac Science, Bothell, Wash.) at a comfortable, self-selected walking speed for 90 s. The subject then stood for 10 s. The point during the 10 s stand at which the subject achieved equal and stable weight-bearing (typically after about 4 s) was considered as the reference limb fluid volume for the session. The basis for using this time point as the reference fluid volume is that in prior investigations it was found that subjects take one sit/stand/walk series to adjust to the electrodes and the socket. The cycle of sit/stand/walk, with no liquid in the bladders, was repeated. Then the subject stood on the electronic scale for 90 s, and the point at which stable equal weight-bearing was achieved was noted. During the subsequent 90 s sit, liquid was injected into the bladders, either 7 ml or 5 ml depending on the practitioner's judgment of how tightly the socket fit. 5 ml was used if the practitioner deemed the fit was particularly tight, and 7 ml was used otherwise. The subject then repeated the sit/stand/walk series. The subject then sat in the chair, and the bladder liquid that was added before the sit/stand/walk series was removed (7 mL or 5 mL). This process was repeated, adding an additional 7 ml (or 5 ml) in each cycle. Thus liquid additions were 7 mL, 14 mL, 21 mL (or 5 mL, 10 mL, 15 mL), and so on. Liquid was added to the bladders until the subject indicated that the socket was uncomfortable. The liquid setting the subject considered most comfortable was also noted. Two sit/stand/walk series with 0 ml bladder liquid were performed in succession at the end of the session.

Analysis—

Body mass index (BMI) was calculated as the quotient of mass (kg) and the square of height (m²). No correction was made to BMI for the lack of an intact limb.

Total limb volume within the region of interest (Volume_{R0I}), which was between the centers of the voltage-sensing electrodes, was calculated using the circumferential (C₁,C₂) and limb length (L) measurements in a truncated cone model, assuming the residual limb cross-section was circular:

$${Volume}_{ROI} ( mL ) = \frac{L}{12 π} [ {C}_{1}^{2} + {C}_{1} {C}_{2} + {C}_{2}^{2} ] \qquad ( 2 )$$

To calculate percentage reduction in socket volume for bladder liquid additions, a total contact socket was assumed in the region of interest. The bladder liquid volume was divided by Volume_{R01}:

$$\begin{matrix}\% change in socket volume \\ for bladder liquid addition\end{matrix} = \frac{bladder liquid addition ( mL )}{{Volume}_{ROI} ( mL )} \qquad ( 3 )$$

To process the bioimpedance data, custom code written in Matlab was used that implemented a Cole model algorithm similar to that used by the commercial instrument manufacturer (v.2.2, XiTRON). Using limb circumference and voltage electrode separation measurements in an accepted geometric model, the data was converted to extracellular fluid volume. In the analysis only data collected during the brief 10 s standing intervals, more specifically the point at the onset of equal weight-bearing, after 90 s walks on the treadmill was used. All data were expressed as a percentage fluid volume change relative to the reference volume (ref), which was taken during the first 10 s standing interval after the first sit/stand/walk series (bladders empty):

$$\% age limb fluid volume change ( t ) = \frac{{V}_{ECF} ( t ) ( mL )}{{V}_{ECF ref} ( mL )} \qquad ( 4 )$$

The percentage limb fluid volume change over the session was calculated. Additionally, the injection slope, defined as the quotient of percentage fluid volume change and percentage change in socket volume for each bladder liquid addition was calculated:

$$injection slope = \frac{\% age limb fluidvolume change}{\begin{matrix}\% age change in socket volume for \\ bladder liquid removal\end{matrix}} \qquad ( 5 )$$

Removal slope was calculated:

$$removal slope = \frac{\% age limb fluidvolume change}{\begin{matrix}\% age change in socket volume for \\ bladder liquid removal\end{matrix}} \qquad ( 6 )$$

A term “limb fluid transport compliance” was created and used to characterize the inability of a limb to recover fluid volume after insert liquid is injected and removed. A high compliance means that the limb does not “bounce back” to its fluid volume before the cycle of bladder liquid injection and removal. The limb fluid transport compliance was defined as the difference between the injection slope and removal slope:

fluid transport compliance=injection slope−removal slope (7)

An exploratory analysis was also conducted to establish relationships between the calculated measures, characteristics of subject health, and the vascular tests.

Results—

Data were collected from a total of 23 subjects. However, data from four subjects were excluded from analysis: One subject had metal orthopedic hardware within the thigh of his residuum which distorted the bioimpedance data. One subject's residual limb length was too short for the bioimpedance electrodes to be spaced properly. For another subject, too long a spacing between voltage-sensing electrodes resulted in impedance measurements outside of the manufacturer's calibration range. One subject used an elevated vacuum socket, and it was not possible to maintain suspension with the bladders in place.

The nineteen participants averaged 54.2 yr (s.d. 10.4) in age, 91.4 kg (s.d. 19.5) in mass, 180.8 cm (s.d. 16.8) in height, and 28.3 kg/m² (s.d. 6.7) in BMI. One subject had bilateral lower-limb amputations, and all other subjects had a unilateral amputation. Time since amputation averaged 14.9 yr (s.d. 14.3), and mean residual limb length was 16.4 cm (s.d. 3.6). Volume in the region of interest (Volume_{ROI}) averaged 990.4 mL (s.d. 290.6). Twelve subjects used an elastomeric liner with locking pin suspension, three used a one-way suction valve, and three used a suspension sleeve. One used a lanyard suspension. Seven subjects had peripheral arterial disease, eleven had high blood pressure, and four were diabetic. Two had congestive heart failure, and one of those individuals also had kidney failure. Twelve subjects had a BMI greater than 25 kg/m², and five of those had a BMI greater than 30 kg/m².

Of the nineteen participants sixteen used four bladders in the study, and three used three bladders. Participants walked with bladder liquid volumes up to 42.0 mL. The mean maximum bladder liquid volume (i.e. before subjects indicated that the socket was uncomfortable and no more liquid should be added) was 26.3 mL (s.d. 8.3). However, the mean maximum bladder liquid volume that subjects preferred was 16.8 mL (s.d.8.4). 16.8 mL (s.d.8.4) corresponded to 1.7% (s.d.0.8%) of the average Volume_{ROI}. There was not a consistent relationship between the percent reduction in socket volume from injecting liquid into the bladders and the resulting percent residual limb fluid volume change. The quotient of percentage change in residual limb fluid volume to percentage reduction in socket volume for bladder liquid additions ranged from −2.5 to 1.0 with a mean of −0.6 (s.d. 0.4) (using nineteen means, one for each subject). Thus on average, for a 1.0% decrease in socket size from injecting liquid, a 0.6% decrease in residual limb fluid volume was observed. For liquid removal, the quotient of percentage change in residual limb fluid volume to percentage change in socket volume ranged from −2.0 to 1.5 with a mean of −0.1 (s.d. 0.4). Thus on average, for a 1.0% increase in socket size from removing liquid, a 0.1% decrease in residual limb fluid volume was observed. So in general not as much fluid returned to the residual limb after bladder liquid was removed as was displaced out of the limb when bladder liquid was added.

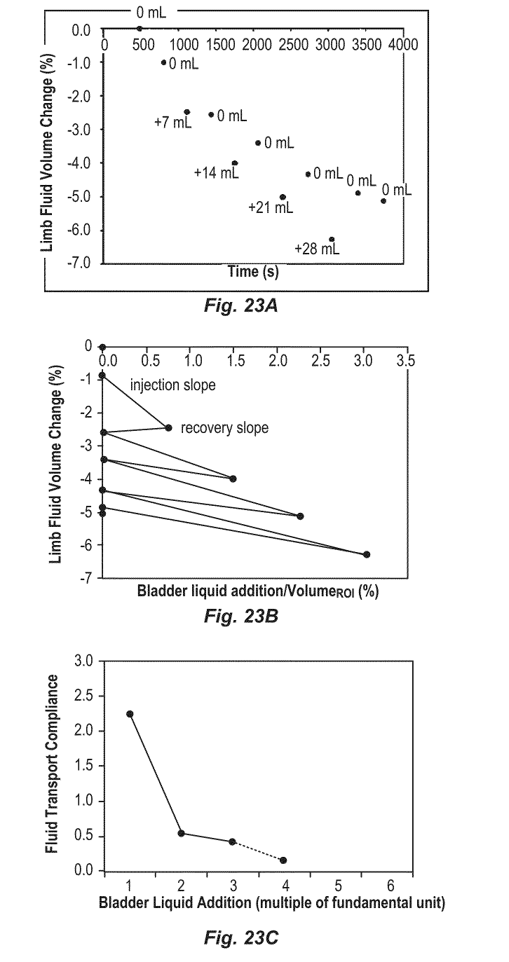

FIGS. 23A-23C show representative results from a subject. FIG. 23A shows the percentage of residual limb fluid volume change for each bladder liquid addition and removal. As can be seen, the subject showed a gradual decrease in fluid volume over the session, with increased loss when bladder liquid was added. FIG. 23B shows a percentage of limb fluid volume change versus the percentage of socket volume change for each bladder liquid addition. FIG. 23C shows fluid transport compliance for each bladder liquid addition.

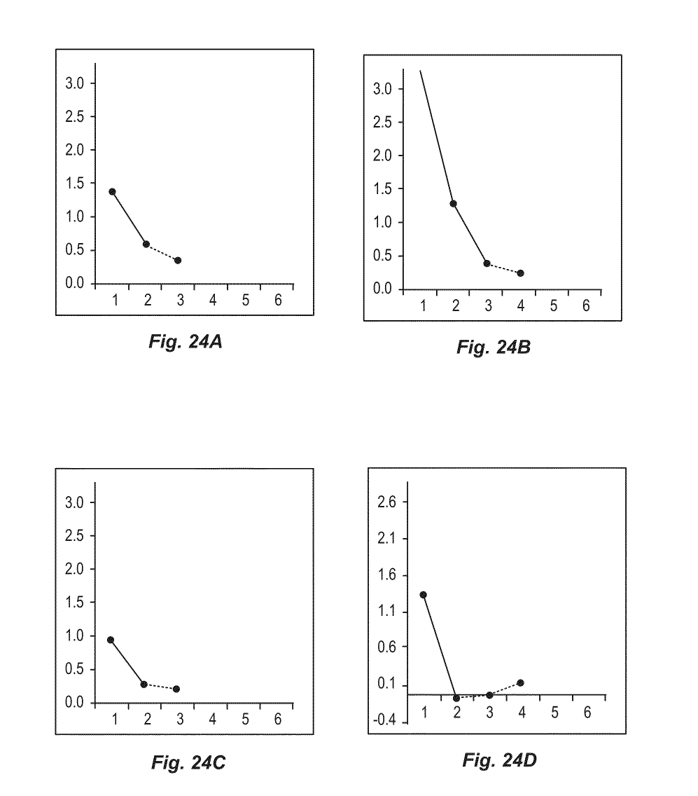

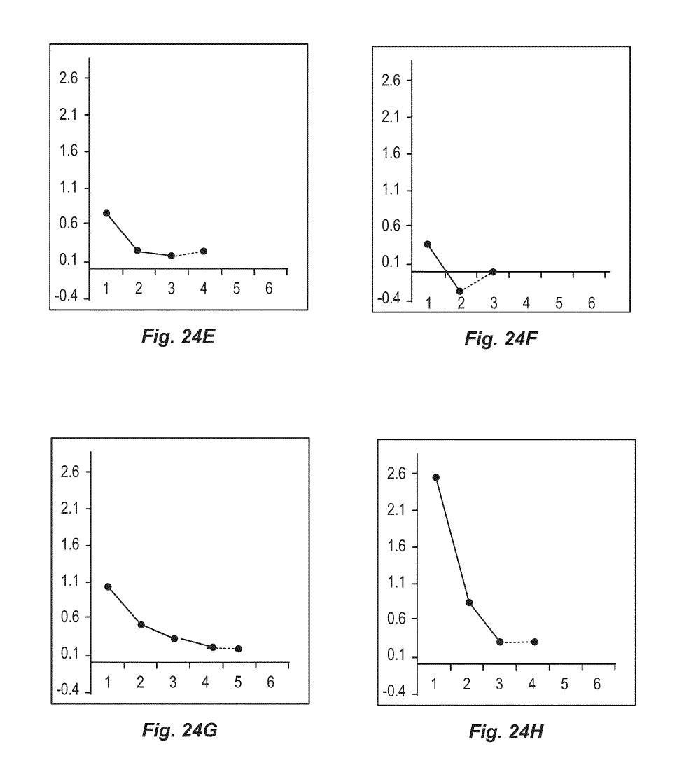

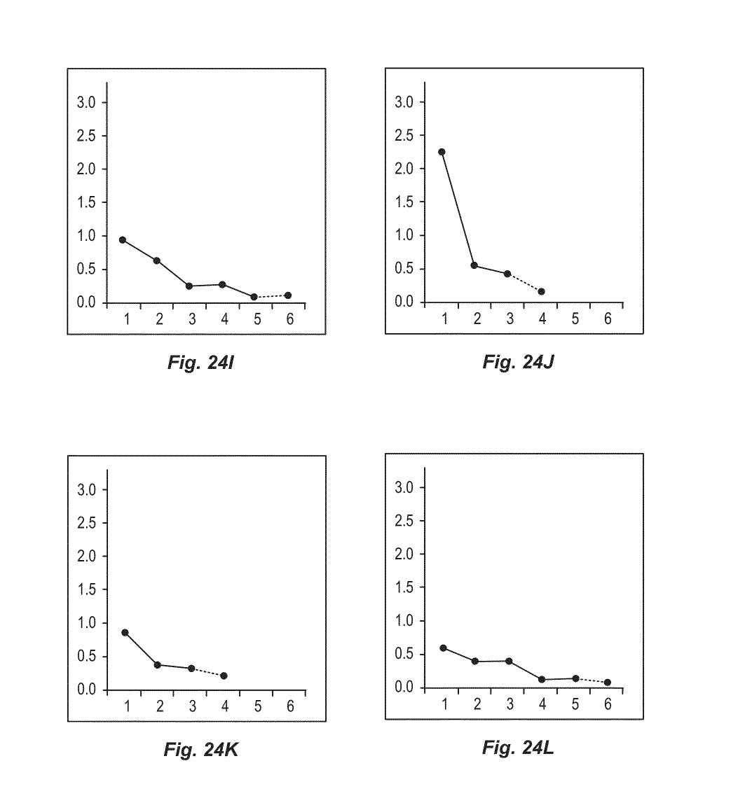

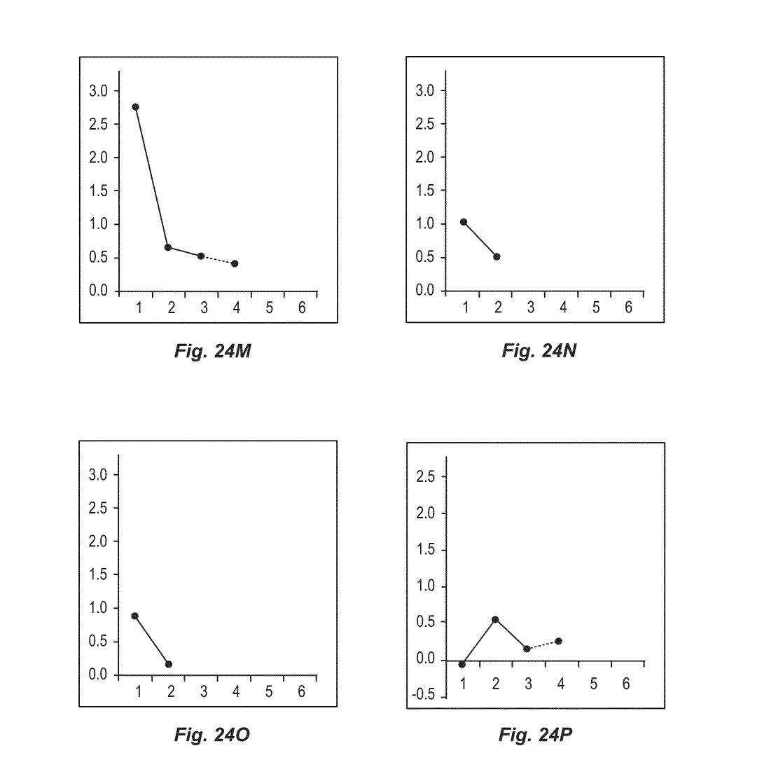

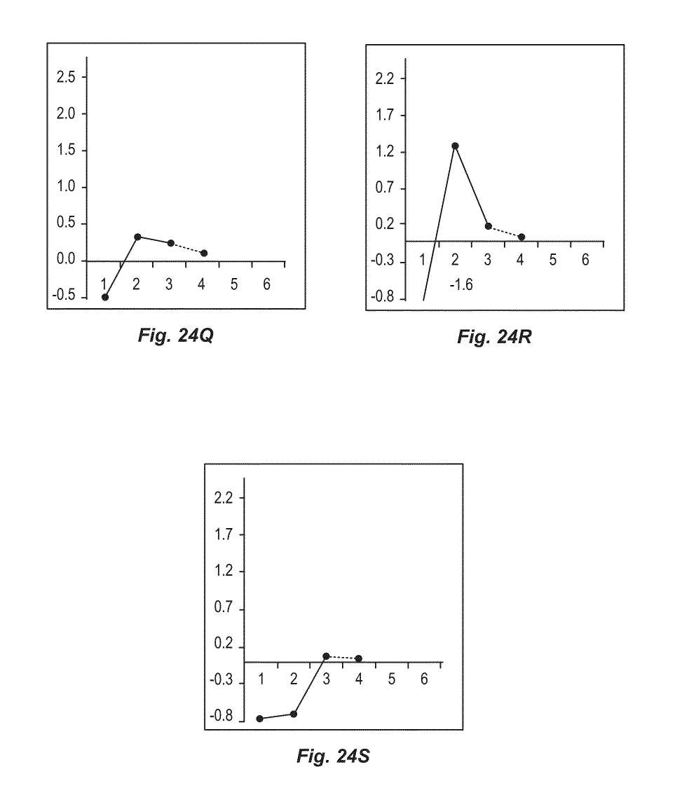

Limb fluid transport compliance data from nineteen participants are presented in FIGS. 24A-24S. Subjects 24A-24I were at the primary inflection point or minimum in their transport compliance curve when the most comfortable bladder liquid setting was reached, indicated in the lot by a transition to a red line. Subjects 24J-24M were beyond the primary inflection point when the most comfortable bladder liquid setting was reached. Subjects 24N-24O experienced a bladder that broke during testing. Subjects 24P-24S showed an initial increase in limb fluid transport compliance, followed by a decrease in transport compliance.

The trend of greater limb fluid volume loss upon liquid injection into the bladders than fluid volume gain upon bladder liquid removal was more accentuated early in the session when bladder injection volumes were low compared with later in the session when bladder injection volumes were high. There was typically a gradual loss in limb fluid volume over the session. For the nineteen subjects limb fluid volume change from the stand after the first walk cycle (defined as the zero reference) to the stand after the last walk at the end of a session ranged from −10.7% to 1.5% with a mean of −3.1% (s.d. 2.9). Only one subject showed a change less than −6.0% (i.e. −10.7%), and one subject showed a change greater than 1.0% (i.e. 1.5%). Limb fluid volume changes from the beginning to end of a session for the rest of the subjects were more evenly distributed. Six subjects had changes between −6.0% and −4.0%, seven subjects had changes between −4.0% and −1.0%, and four subjects had changes between −1.0% and 1.0%. The fluid volume changes at the preferred bladder liquid volume, excluding the two subjects for whom a bladder ruptured, averaged −3.5% (s.d. 2.7).

Limb fluid transport compliance, defined as the difference between the injection slope and removal slope (%/%) in the fluid volume change vs. socket volume change plot (slopes defined in FIG. 23B) typically reduced with greater liquid volume injected into the bladders (FIGS. 24A-S). A fluid transport compliance value of 0.0 indicates complete limb fluid volume recovery within a bladder injection/removal cycle, while a positive value increasingly further from 0.0 indicates less recovery within an injection/removal cycle. A negative value indicates less fluid volume was lost after bladder liquid was injected than was recovered after bladder liquid was removed (edematous limb).

For the fifteen subjects who demonstrated residual limb fluid volume reduction when liquid was injected into the bladders, the bladder liquid volume for which the socket was most comfortable was not at a limb fluid transport compliance value of 0.0, i.e. complete elastic recovery (see FIGS. 24A-24O). However, more than half of the fifteen subjects (nine subjects) had reached or were at an inflection point or minimum in their fluid volume fluid transport compliance vs. injection cycle plot when the most comfortable bladder liquid volume was reached (see FIGS. 24A-24I). For five subjects, the most comfortable bladder liquid volume was at a point after the inflection point or minimum (see FIGS. 24J-24M). For two subjects fluid volume for which the socket was most comfortable could not be determined because the bladder broke during the test (see FIGS. 24N, 24O). The limb fluid transport compliance at the most comfortable setting for all subjects except the two for whom the bladders broke ranged from 0.0 to 0.6 with a mean of 0.3 (s.d. 0.2). Thus subjects were still losing some fluid volume at what they considered their most comfortable setting.

Four subjects, unlike the other fifteen subjects, demonstrated limb fluid volume increases upon injection of liquid into the bladders for low bladder liquid volumes at the beginning of the session (see FIGS. 24P-24S). However, for greater bladder liquid volumes, limb fluid volume decreased upon injection of bladder liquid like it did for the other subjects (see FIGS. 24A-24O). These four subjects and only these four subjects demonstrated presence of venous insufficiency in their contralateral limb (ASGP testing).

Discussion—

The maximum bladder liquid volume subjects tolerated ranged to 42.0 mL. However, the preferred bladder liquid volume was 16.8 mL (s.d. 8.4), corresponding to 1.7% (s.d. 0.8%) of the average socket volume in the region of interest. The point the prosthesis user preferred bladder liquid volume during ambulation was not at a point where fluid out equaled fluid in for an inject/remove cycle. There was not a consistent relationship between the percent reduction in socket volume from injecting liquid into the bladders and the resulting percent residual limb fluid volume change. This result held across subjects, and for different bladder liquid additions on the same subject. Limb fluid volume driven out of the residual limb when bladder liquid was added was typically not recovered upon subsequent bladder liquid removal.

The results of the study showed that each subject's limb fluid volume loss/bladder liquid addition relationship is different—fifteen of nineteen subjects experienced a gradual limb fluid volume loss over the test session whereas four of the nineteen subjects initially experienced fluid volume gains when bladder liquid was added. Thus it may be advantageous to provide socket volume control strategies on a case by case basis depending on the subject type.

The results from the study also suggest that fluid volume driven out of the residual limb as a result of adding liquid to the bladders was not easily recovered when the bladder liquid was removed. Accordingly, it may be beneficial for a controller to be designed so that socket volume is small enough that good suspension is achieved while avoiding smaller socket volumes that run the risk of increasing limb fluid-volume loss over time.

Further, this behavior may be an important consideration for engineers designing adjustable socket technologies and for practitioners fitting these systems to patients. It may not be reasonable to assume that one bladder volume will induce only one limb fluid volume, even within the same day. Thus, a controller may be designed to use a dynamic set point of bladder liquid volume during ambulation (more liquid may be required with longer periods of activity).

However, if increasing bladder liquid volume reduces residual limb fluid volume further, then more bladder liquid may need to be added, either manually or automatically, in order to compensate. It would be undesirable for a residual limb to enter a positive feedback loop where further limb fluid volume loss occurs, requiring further bladder liquid to be added, and the cycle repeated until excessive pressure is applied to the limb. The excessive pressure and smaller socket volume may run the risk of increasing limb fluid-volume loss over time. If the residual limb reduces in volume when socket size is reduced and is not allowed to recover, through either passive or active actuation, then the residual limb could become dehydrated and possibly atrophy over the long term. Accordingly, excessive pressures should be avoided.

Periods of limb fluid volume recovery or some other treatment may be beneficial to retard continued fluid volume loss. A controller that implements recovery strategies such as enlarging the socket during rest periods, may be advantageous and facilitate fluid volume recovery and reduce daily fluid volume loss. The controller may have a dynamic set point of bladder liquid volume but within set limits so that excessive pressure is not applied to the limb, and instability is not induced.

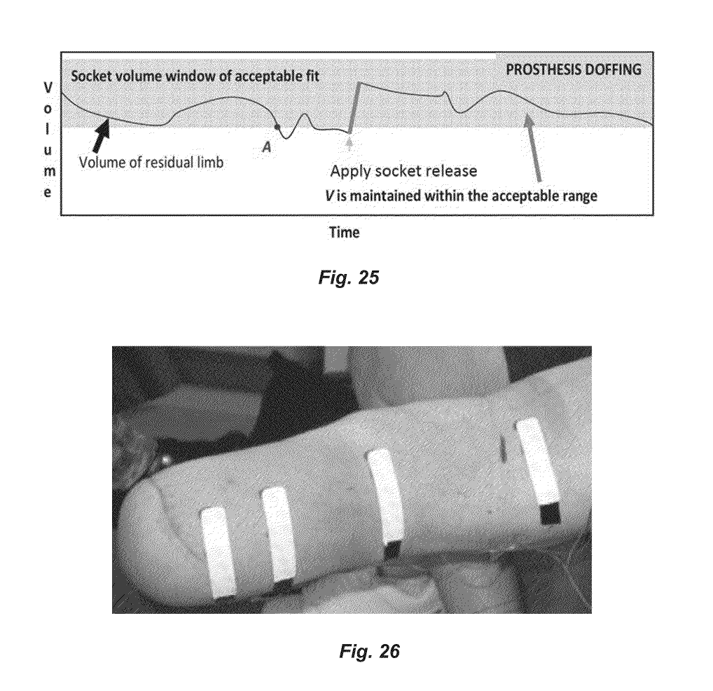

Thus improved controller strategies may be provided that control socket volume while the person ambulates with the prosthesis as well as facilitate limb fluid volume recovery. One option for fluid volume recovery is while the person is not weight-bearing on the prosthesis. FIG. 25 illustrates an exemplary concept of fluid volume recovery. The intent of applying socket release is to relieve pressures on the residual limb so that fluid volume returns and helps to keep limb volume within the window of acceptable fit.

### Recovery Strategy Research

Studies were conducted to determine whether fluid volume recovery could be induced by removing the prosthesis.

Methods—

Volunteers were considered for inclusion if they had a trans-tibial amputation more than 12 months prior and were at a Medicare Functional Classification Level (MFCL) of K-2 of higher (at least a limited community-level ambulatory). Subjects were required to use a prosthetic limb for an average of at least 4 hours per day, determined by self-report, and capable of treadmill walking at a self-selected walking speed for at least 5 minutes. Subjects were not included if they were currently experiencing skin breakdown or if their residual limb length did not allow at least a 5.5 cm distance between the voltage-sensing electrodes (described below). Human subject approval from a University of Washington Internal Review Board was granted and informed consent was obtained before any study procedures were initiated.

Subjects were asked to refrain from consuming alcohol or caffeine on the day of testing. After arriving at the lab, the subject wore the prosthesis while mass and height were recorded. The subject sat for 10 minutes while the research practitioner queried the subject about smoking habits, presence of diabetes, and prosthesis history.

A multi-frequency bioimpedance analyzer (Hydra 4200, Xitron, San Diego, Calif.) was used to assess residual limb extracellular fluid volume. Four electrodes (Xitron, 77×20 mm contact surface, 0.81 mm thickness) were used. A thin layer of ultrasonic coupling gel (Couplant D, GE Panametrics, West Chester, Ohio) was placed underside each electrode. Tegaderm™ (Transparent Film Dressing, 3M, St. Paul, Minn.) was used to strain relieve wires to the electrodes. A custom, four-pin, Delrin, flat connector (9.0 mm×11.5 mm, 2.5 mm thickness) was designed that accommodated gold-plated pins (WPI, Viking Electronics, Inc. (division of Cooper Interconnect), Moorpark, Calif.) to attach the four insulated lead wires from the Xitron instrument cable to the electrodes. This enhancement ensured a stable and consistent signal was recorded while the subject walked on the treadmill.

The subject doffed the prosthesis, and the skin was prepared for the bioimpedance electrodes. The skin was rubbed gently with sandpaper (Red Dot™ Trace Prep 2236, 3M, St. Paul, Minn.) to achieve good electrical coupling. Two outer electrodes injected current while two inner electrodes sensed voltage (see FIG. 26). The proximal voltage-sensing electrode was positioned at the level of the patellar tendon proximal to the fibular head. The distal current injecting electrode was placed as far distally as possible but still on the curved cylindrical portion of the residual limb. The distal voltage-sensing electrode was positioned at least 3.5 cm proximal to the distal current-injecting electrode and always proximal to the distal end of the tibia. The proximal current-injecting electrode was placed at least 7.0 cm proximal of the proximal voltage-sensing electrode such that it was outside of the socket brim but under the liner or suspension sleeve. Care was taken to ensure no loss of suction from air escaping along the lead wires extending out at the thigh from under the liner or sleeve. The Xitron instrument applied current at between 50 μA and 700 μA across 50 frequencies (5 kHz to 1 MHz) each second, and measured amplitude and phase differences between the injected and sensed signals at a 1 Hz sampling rate. The bioimpedance data was plotted in approximately real time (3 s delay) using custom Matlab (Mathworks, Natick, Mass.) code that implemented a Cole model, similar to that used in the Xitron post-processing program, so that set up problems could be identified if they existed.

After the electrodes were applied and the instrument was started, the subject donned the prosthesis, standing and weight-bearing for several seconds until comfortable. Three different protocols were executed with no order preference except that the Walk protocol was conducted first or last. Fewer electrode mechanical problems were experienced if the Walk protocol was conducted first or last.

(1) Sit Protocol: The subject sat for 10 minutes in a stable chair with the prosthesis donned and the foot supported on the floor. Care was taken to ensure good sitting posture, since too much knee flexion may occlude blood flow and too much extension may cause a slouching posture. Typically to achieve good posture, the subject's knee was in approximately 100 degrees of extension. Then the subject sat and doffed the prosthesis, socks (if worn), and liner, and then sat for 10 minutes longer.

(2) Liner Protocol: Same as Sit Protocol except the liner was maintained during the final 10 minutes sitting period.

(3) Walk Protocol: The subject underwent repeated cycles of sitting, standing, and walking at his or her self-selected walking speed with approximately ⅓ of the time spent sitting, ⅓ standing, and ⅓ walking. The total duration was approximately 30 minutes, and a walking cycle of 3 to 5 min duration was conducted last so as to elevated vascular flow at doffing. The subject then sat and doffed the prosthesis, socks (if worn), and liner and sat for 10 minutes longer.

Using standard medical testing procedures, orthostatic blood pressure (OBP) was measured on the day of the test, and on a different day ankle brachial index (ABI) and segmental limb pressure (SLP) were assessed. An electronic blood pressure measurement unit (HEM-775, Omron, Kyoto, Japan) was to determine OBP. A commercial cuff inflator (TD312 Cuff Inflator, MV10 Manifold Selector, and SC12 and SC10 cuffs, Hokanson, Bellevue, Wash.) and a Doppler flow meter (MD6 Doppler, Hokanson) were implemented to evaluate ABI and SLP. Collected data were interpreted for presence of high blood pressure and arterial disease by a practicing endocrinologist using standard clinical procedures. Subject health records were consulted to identify presence of a major medical condition (e.g., congestive heart failure, kidney failure, diabetes, cancer).

Body mass index (BMI) was calculated as the quotient of mass (kg) and the square of height (m²). Because the subjects wore their prosthesis while mass was measured, no correction was made to BMI for the lack of an intact limb.

The bioimpedance data was processed using custom code that implemented a Cole model algorithm similar to the manufacturer's (v.2.2, Xitron). The data was converted to extracellular fluid volume using limb circumference and segment length measurements in a well-accepted geometric limb model. Using the fluid volume measured immediately after doffing as a reference, the percentage fluid volume change over time was calculated during the 10-minute sitting period. Percentage fluid volume change was presented instead of fluid volume in mL because of the dependence of the results on limb length and size. Use of percentage fluid volume change normalized the data so that comparisons among subjects could be made.

Descriptive analyses (summary statistics and visual displays) were performed for all variables. The linear association between variables was assessed by Pearson correlation. Due to the exploratory nature of the study and the small sample size, the data analysis focused on exploratory and descriptive methods.

Results—

A total of 22 males and 8 females participated in this study. Their mean age was 50 years (s.d.13). Twenty subjects had their limb amputation as a result of trauma, seven from vascular disease, one from Larsson's syndrome, one from cancer, and one from spina bifida. All but three subjects had a unilateral amputation. Subject mass averaged 90 kg (s.d. 20), height averaged 177 cm (s.d. 10), and BMI averaged 28.7 (s.d. 5.9). Eleven subjects were obese (defined as BMI>30) and another six subjects were overweight (defined as BMI>25). Ten subjects were at a MFCL of K-2, fourteen at K-3, and six at K-4. Nine subjects were diabetic, 20 were not, and one subject's diabetic status was not known. Eight subjects were smokers, and 22 were not. Eighteen subjects used an elastomeric liner with pin suspension, while twelve subjects used a different type of suspension.

Fourteen subjects had high blood pressure, fifteen did not, and one subject's status was not known. Twelve subjects had an ankle brachial index (ABI) indicative of peripheral arterial complications, fifteen did not, and one subject's status was not known.

All test sessions were started between 9:30 am and 2:00 pm. Tests were started during morning hours (before noon) for sixteen subjects, and after noon for fourteen subjects.

How much does residual limb fluid volume change after doffing, and how long does it take to stabilize? Residual limb fluid volume change after doffing ranged from −1.1% to 8.3% with a mean of 1.9% (s.d. 1.6). The time to achieve peak fluid volume ranged from 36 seconds to 10.0 minutes with a mean of 6.0 minutes (s.d.4.0).

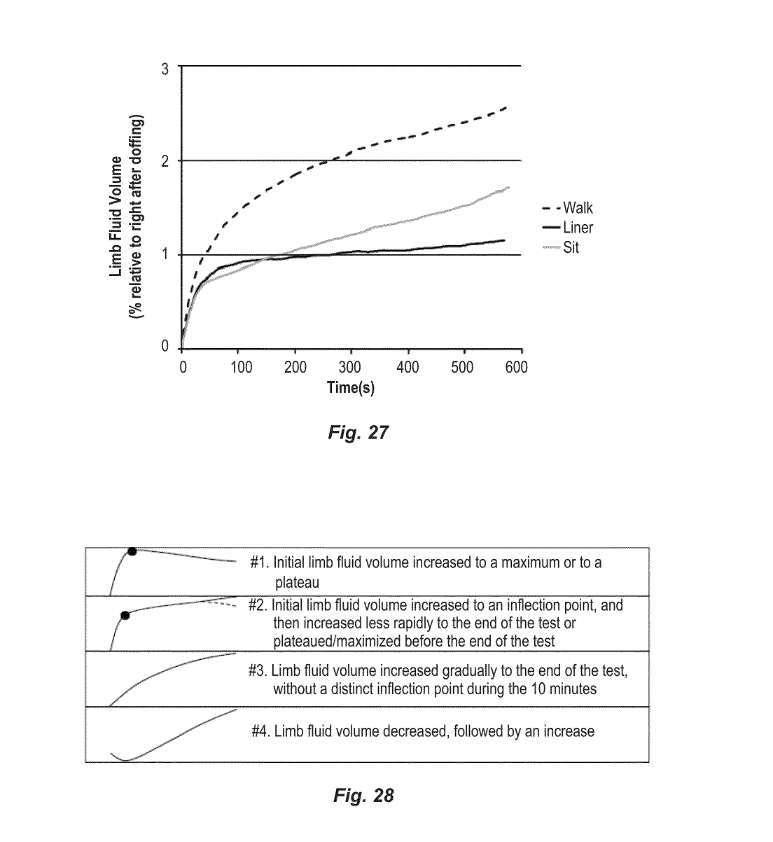

The shape of the response curve depended on the test performed. In general, for all three tests (Sit, Liner, Walk) limb fluid volume increased quickly initially, during the first minute after doffing, and then increased less quickly afterwards (see FIG. 27). FIG. 27 shows the residual limb fluid volume change over time after doffing. Average response curves for all subjects are shown for the three test protocols: Sit, Liner, and Walk. The initial rate of increase tended to be higher for the Walk test than the Sit or Liner tests. Subsequent limb fluid volume rates of change were comparable for the Sit and Walk tests but not the Liner test. The Liner response curves tended to stabilize towards a plateau rather than continue increasing. On average, at one minute after doffing, at least 40% of the total limb volume change for the 10 minutes period was accomplished for all three tests.

Is fluid volume change different after walking than after sitting? The fluid volume change after Walk, mean of 2.8% (s.d. 2.0), was significantly different from that after Sit, mean of 1.8% (s.d. 1.4) (p=0.03). However, the time to reach a stable fluid volume was not significantly different between Walk and Sit (p=0.79) (TABLE 2).

TABLE 2
Comparison of Walk and Sit test results

| | Time to max or | |
|---|---|---|
| | stable fluid volume | % fluid volume change |
| Walk | 6.9 minutes (s.d. 3.7) | 2.8% (s.d. 2.0) |
| Sit | 6.6 minutes (s.d. 3.8) | 1.8% (s.d. 1.4) |
| p-value | 0.79 | 0.03 |

Is the amount of limb fluid volume increase reduced when a liner is maintained after doffing compared with no liner? The fluid volume change from the Liner test was not significantly different from that of the Sit test (p=0.13). However, the time to achieve a stable fluid volume for Liner, mean of 4.3 minutes (s.d. 4.1), was significantly different from that for Sit, mean of 6.6 minutes (s.d. 3.8) (p=0.03). Thus limb volume stabilized more quickly with the liner present.

Exploratory analysis was conducted to investigate if the magnitudes of limb fluid volume change and the time they were achieved were related to qualities of the subjects and prostheses. The following qualities showed no significant correlation (using a test statistic of 0.05): subject gender, BMI, presence of peripheral arterial complications, high blood pressure, diabetes, or smoking. However, there were common shapes in the response curves for different subjects. Thus further investigation was conducted to determine if there were relationships between curve shapes and features of the subjects and prostheses.

The results were grouped according to the four curve shapes illustrated in FIG. 28. FIG. 28 shows the shapes of post-doffing response curves. Curve shapes #1 and #2 demonstrated a concave inflection point soon (e.g., less than two minutes) after doffing. Curve shapes #3 and #4 lacked this characteristic.

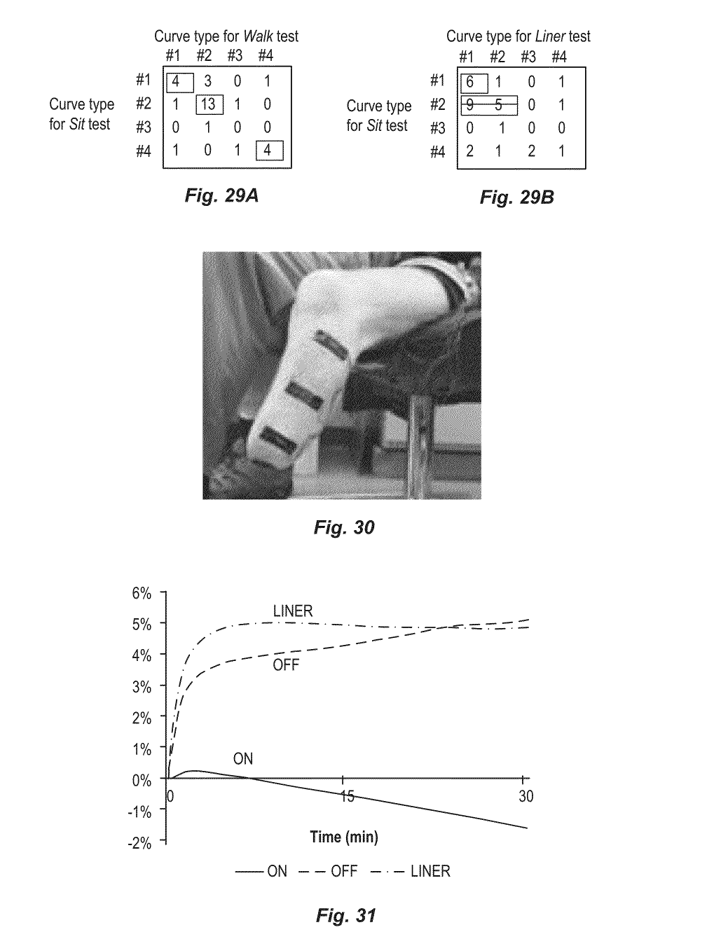

FIG. 29A and FIG. 29B summarize the curve type results. The number of subjects with each combination of curve types are shown. Boxes with high values are highlighted. Subjects tended to have the same type of curve for Sit and Walk, and most of those were type #1 or #2 curves (FIG. 29A). A total of 21 of the 30 subjects had the same curve type for the Walk test as for the Sit test. For Sit vs. Liner, however, subjects with type #2 curves for Sit tended to switch to type #1 curves for Liner (FIG. 29B). Subjects with type #4 curves for Sit tended to switch to a lower number curve for Liner.

Subjects with curve types #2 and #3 tended to have higher magnitude volume changes than subjects with curve types #1 and #4, whether for Sit, Liner, or Walk test condition. Thus the shape of the curve was associated with a greater or lesser magnitude of volume change.

86% of subjects with type #3 and #4 curves tended to be overweight or obese, while only 48% of subjects with type #1 and #2 curves were overweight or obese. Subjects who used liners with locking pins tended to have curve types #1 and #2 (74%), while fewer subjects with types #3 and #4 had locking pins (20%).

Discussion—

The residual limb fluid volume increase that occurs after doffing is relevant to clinical practice and advancing limb prosthetics. Post doffing volume change can affect the shape of the cast or the image scan that is used as a starting shape for socket design. By understanding how much and over what time course a residual limb will increase in fluid volume upon doffing, controllers may be able to better factor the expected percent increase into the socket modification to ensure a proper socket fit. It may be possible to develop best practices to facilitate socket design.

The residual limb typically enlarges after doffing because of a change in interstitial fluid pressure. While the residual limb is within the socket, interstitial pressure is elevated because of the pressure applied by the socket wall on limb soft tissues. When the socket is doffed, however, this constraint is released and interstitial fluid pressure decreases. As a result, the arterial to interstitial pressure gradient increases, and more fluid enters the interstitial space. The interstitial to venous pressure gradient decreases, reducing fluid transport from the interstitium into the venous vasculature out of the limb. Further, the reduction in interstitial tissue pressure may cause vessels to enlarge and more blood to enter the residual limb. Thus both interstitial fluid and blood may contribute to the fluid volume increase after doffing.

Residual limb fluid volume increased more after walking than after sitting presumably because of the increased arterial fluid drive after exercise. The increased arterial drive caused a greater blood volume flow rate through the vasculature. Pressure-induced vasodilation may be at work here. There may be a greater driving force to move fluid across from the arteries into the interstitial space and as a result limb fluid volume may increase more after walking than after sitting.

For subjects who experience limb volume loss over the day, a means to help increase volume back up to levels experienced earlier in the day may be to remove the prosthesis temporarily right after walking Controllers that provide doffing intervals of appropriate duration and timing may help the subject keep limb volume within an acceptable range and negate the need for sock addition or other accommodation. Further, if sockets were designed to increase in volume during rest after activity then limb volume over the day may stabilize.

Because the liner may restrict limb fluid volume increase and keep interstitial pressure elevated (though not as elevated as with the socket donned), the subject leaving the liner on tended to stabilize limb fluid volume faster than the subject removing the liner. Interestingly, while keeping the liner on influenced the time to stabilization, it did not influence the volume at stabilization compared to without wearing the liner (Sit condition). This result is somewhat surprising. It suggests that liners are too stretchy to strongly control limb fluid volume; their impact is more moderate. If this result held after walking then it would provide supporting evidence for “dynamic” sockets, sockets that adjust volume according to activity of the user. A socket design that enlarged after walking may facilitate fluid volume recovery and thus reduce the need to accommodate, i.e. the need to remove the socket to add socks. Eliminating the need to remove the socket would increase convenience over the current common accommodation practice of adding socks.

Subjects had different shaped post-doff curves because they have different fluid transport capabilities. Decreases upon doffing (group #4 curve shapes) may be due to co-morbidities. The initial decrease may have reflected the time needed to increase local arterial pressure high enough to open occluded arteries. The faster volume increase on most subjects, may reflect primarily blood volume returning to the limb, while the later part, a slower volume change, may reflect primarily interstitial fluid increase. The interaction and magnitudes of these two flow mechanisms may dictate the shape of the post doff volume curve. Overweight and obese subjects tended to have type #3 and #4 curves; they lacked a concave inflection point after doffing but instead showed gradual changes in fluid volume change. This result may reflect complications associated with being overweight, for example a delayed vascular response. The lack of a locking pin might have accentuated this effect since a locking pin tends to pull distal limb tissues distally during swing phase and may accentuate interstitial fluid draw into the residual limb.

Part of why pursuing the shape of the post-doffing response curve is meaningful is that commercial imaging systems may be capable of collecting these data. If relevant relationships between socket fit and post-doffing volume results were found then those findings could be applied directly to clinical practice to help improve patient care.

Clinically, for the practitioner to capture a residual limb shape of volume closest to ambulatory limb volume, casting or scanning right after doffing may be advantageous. Notably, the residual limb will be increasing in volume. Fast scanning procedures, for example optical scanning, may reduce measurement error. An alternative strategy is to wait for stabilization (see Liner in FIG. 27) and then attempt to factor the expected percent limb volume increase into the modification.

The results from the present study suggest that practitioners preparing to cast or image a residual limb for socket design should have their patient sit quietly for 10 minutes before doffing. Patient history and fit of the current prosthesis can be discussed during this 10 minute period. In the present study this quiet interval reduced fluid volume increase about 36% compared with walking before doffing. It also reduced continued limb expansion over the subsequent 10 minutes.

Residual limb fluid volume may stabilize faster if the liner is maintained rather than removed. A stable limb shape may be important when using imaging systems that take more than a few seconds to image the limb, for example laser scanners. Imaging systems that acquire residual limb shape quickly and right after doffing should be encouraged so as to reduce the detrimental impact of limb fluid volume change on limb shape measurements.

An adjustment controller may be configured to release liquid volume right after sitting so as to induce limb fluid volume recovery. The controller could be configured to detect a user's intent to sit and then release the socket in such a way that the prosthesis does not apply excessive pressures to the limb but at the same time does not fall off the limb.

Residual limb fluid volume recovery after doffing is potentially a major source of limb fluid volume recovery. The changes to 8.3% measured in this study are much greater than the losses deemed clinically significant, which are as low as 1%. Advantageously, a controller that could release the socket in such a way that it did not need to be removed but at the same time facilitated fluid volume recovery may provide a recovery strategy to counter limb fluid volume losses during activity.

The initial quick rise in fluid volume recovery after doffing and releasing may be the ideal time to apply socket release because the change per unit time is so high. Thus some controllers and/or accommodation sockets may benefit from a quick-release strategy.

Presence of the locking pin tended to facilitate a type 1 or 2 curve, which suggests improved fluid volume recovery; pulling on the soft tissues distally may facilitate recovery, suggesting that distal suction or vacuum application during swing phase facilitates fluid volume recovery. Active mechanisms configured to pull fluid back into the limb as soon as the person sits, that then subsides may be beneficial.

A liner may be sufficient to stabilize limb fluid volume increase. Thus if the hoop stress in the liner is controlled properly then limb fluid volume increase may be potentially limited while the person is sitting with the prosthesis doffed and edema may be potentially avoided for patients prone to edema.

This paper sets some time estimates for the recovery technology—4 to 6 min, depending upon the configuration.

Once most of the recovery is achieved the socket volume may be adjusted so that the prosthesis stays on the limb but does not induce limb fluid volume reduction. The controller may operate based on feedback from sensors within the socket or socket liner to ensure excessive limb swelling or reduction is identified and managed.

### Fluid Volume Recovery Maintenance Research

Studies were conducted to determine if fluid volume recovery achieved during sitting was maintained subsequently after the subject stood and walked wearing the prosthesis.

Participants:

Participant volunteers were included in this study if they had a trans-tibial amputation at least 1 year prior and were using a definitive prosthesis. Participants were required to be capable of at least 2 minutes of continuous ambulation on a treadmill, and 2 minutes of continuous standing with equal weight-bearing. An additional inclusion criterion was a residual limb length between the patellar tendon and distal limb of at least 9 cm (necessary for proper bioimpedance measurement). Participants were not included if they were currently experiencing skin breakdown, or if they had metal implants within their limb. Metal implants can distort bioimpedance data.

Instrumentation:

A custom biompedance analyzer constructed specifically for testing fluid volume changes on people with limb loss was used. The system generated short bursts of controlled sinusoidal electrical current (˜300 μA) at 30 frequencies between 5 kHz and 1 MHz. A collection of 24 bursts for the 24 frequencies, termed a “sweep,” was sent through the residual limb every 40 ms. The current was delivered via electrodes positioned proximally on the thigh and distally on the inferior surface of the residual limb (FIG. 30). Voltage was sensed via four channels, two from the anterior surface and two from the posterior surface. On each aspect (anterior and posterior), one channel measured voltage between one electrode positioned at the level of the patellar tendon and another electrode on the most distal aspect of the cylindrical part of the residual limb. The other channel measured voltage between the electrode positioned at the level of the patellar tendon and another electrode positioned mid-limb (FIG. 30). The custom instrument demodulated the current and voltage and then calculated mean Real and Imaginary demodulation components for each sweep. Data were sent via a 3-m long cable to a stationary computer housed within a portable cart. The cart (MX2-OS-SL-PT-11931, CompuCaddy, Louisville, Ky.) was equipped with a 12V 35A-h lead acid battery, allowing the unit to be electrically isolated.

The entire testing protocol was recorded by video camera so that it could be reviewed later to capture verbal information provided by the subject and research practitioner (Taser Axon, Taser International Incorporated, Scottsdale, Ariz.).

Electrodes were made from a conductive polymer (ARCare 8881, Adhesives Research Inc., Glen Rock, Pa.) (thickness 0.09 mm) and hydrogel (KM10B, Katecho, Des Moines, Iowa) (thickness 0.76 mm). A very thin layer of coupling agent was used between the hydrogel and skin (ultrasonic coupling gel, GE Panametrics, West Chester, Ohio). Multi-stranded silver-plated copper wire with an aramid core and poly vinyl chloride insulation (32 AWG, New England Wire, Lisbon, N.H.) (thickness 0.76 mm) was sandwiched between two pieces of the conductive polymer, and extended out the side of the electrode. Wires were strain relieved using Tegaderm (Transparent Film Dressing, 3M, St. Paul, Minn.) of thickness 0.03 mm.

Test Procedure:

Participants underwent a series of vascular assessments prior to bioimpedance testing (<12 months prior). Using techniques described in detail previously, participants were tested for presence of high blood pressure using orthostatic blood pressure (OBP) assessment. The presence of peripheral arterial disease was tested for using segmental limb pressure and ankle-brachial index (ABI) measurements. An electronic blood pressure measurement unit (HEM-775, Omron, Kyoto, Japan) was used for OBP testing, and a commercial segmental limb pressure measurement system (TD312 Cuff Inflator, MV10 Manifold Selector, and SC12 and SC10 cuffs, Hokanson, Bellevue, Wash.) was used for segmental limb pressure and ABI testing. Collected data were interpreted by a practicing endocrinologist using standard clinical procedures.

On bioimpedance test days, after arriving at the lab the participant sat for 10 minutes to achieve a homeostatic condition and to answer questions about recent health history and changes to their prosthesis. The participant then doffed the prosthesis, socks (if worn) and liner, and the research practitioner inspected the limb for signs of injury or poor prosthetic fit. If the participant was currently experiencing skin breakdown or if socket fit was not acceptable for regular prosthesis use then the participant was referred to his or her regular practitioner. Otherwise, the skin (Red Dot Trace Prep, 3M) was cleaned at sites electrodes were to be placed, and put the electrodes on the residual limb. If a participant had bilateral trans-tibial amputation then the residual limb expected to produce the strongest bioimpedance signal (least skin scarring near electrodes, longest length) was selected for testing. Wires from the electrodes extended proximally up the lateral aspect of the thigh. Care was taken to ensure wires were flat next to each other and then covered with Tegaderm at the brim to ensure that no air escaped along the electrode wires that might cause a loss of suspension.

After the electrodes were placed on the limb and bioimpedance data collection initiated, the participant donned the prosthesis and then sat in a chair for 90 s. Care was taken to ensure a proper sitting posture. Biompedance data were presented in approximately real time (2-3 s delay) to allow the researchers to determine if the electrodes were performing properly. Then the participant stood for 90 s on a platform with an electronic scale embedded in the surface so that weight bearing on the prosthetic limb was monitored. If the participant's weight bearing on the prosthetic limb deviated by more than 10% of half the body weight, then the participant was instructed to shift his or her weight accordingly. Then the participant walked on a treadmill at a self-selected walking speed for 5 min, followed by a brief standing period of less than 10 s on the platform with the embedded electronic scale. The participant then repeated the sit/stand/walk cycle two more times for a total of three sit/stand/walk cycles. The participant then sat down and underwent 30 min sitting with the prosthesis in one of three configurations (conditions): (1) OFF: prosthesis and liner doffed; (2) ON: prosthesis left donned; (3) LINER: prosthetic doffed but liner left donned. Then the subject donned the prosthesis (if it was removed) and conducted three more sit/stand/walk cycles as conducted earlier in the test session. At the conclusion of the protocol the subject doffed the prosthesis, bioimpedance data collection was terminated and the electrodes were removed.

Analysis:

Demodulated bioimpedance data stored to disk were processed using a Cole model. Data out from the Cole model were converted to extracellular fluid volume using a limb segment model. Fluid volume change during the 30-minute rest period for each configuration was quantified, and short-term recovery and long-term recovery were calculated. These three variables were expressed in percentages relative to a consistent reference using the equations below:

$$\begin{matrix}change during 30 minute rest period = \frac{{Vol}_{end sit} - {Vol}_{begin sit}}{{Vol}_{after walk \#3}} \times 100 & ( 8 ) \\ short term recovery = \frac{{Vol}_{after walk \#4} - {Vol}_{after walk \#3}}{{Vol}_{after walk \#3}} \times 100 & ( 9 ) \\ long term recovery = \frac{{Vol}_{after walk \#6} - {Vol}_{after walk \#3}}{{Vol}_{after walk \#3}} \times 100 & ( 10 )\end{matrix}$$

The basis for using Vol_{after walk #3} as the reference for all three calculations is that the protocol was identical up to this point for all three conditions. The effects of the intervention strategy (condition) were of interest relative to this point. The protocol was identical for all three conditions before this point.

An analysis of variance (ANOVA) was conducted to address the following questions. For the three different test conditions (ON, OFF, LINER):

Are there differences in fluid volume change during the 30-minute rest period?

Are there differences in short-term recovery?

Are there differences in long-term recovery?

For the three different test conditions (ON, OFF, LINER):

Are there differences between anterior and posterior results for any of the three measures—rest period fluid volume change, short-term recovery, or long-term recovery?

Results—

Sixteen individuals participated in the study. Two of the sixteen participants did not complete the LINER test; one did not normally wear an elastomeric liner, and one became bedridden before completion of the LINER test. These two participants were included only in the OFF and ON analysis. Of the sixteen participants, fourteen had a unilateral amputation and two had bilateral amputation. Three participants were at a K-2 level, six at K-3, and seven at K-4 (Medicare Functional Classification Level). Two participants had diabetes, seven had high blood pressure, two had peripheral arterial disease, and four were smokers. Eleven were overweight (BMI>25) and four of those eleven were obese (BMI>30). Three of the sixteen participants were female. The average age of participants was 51.9 (s.d.=13.2) years. Time since amputation ranged from 1 to 52 years with a mean of 17.9 years. Mean BMI was 28.6 (s.d.=5.5) kg/m2, uncorrected for differences in prosthetic mass as compared to anatomical limb mass. Mean mass was 91.6 (s.d.=16.3) kg measured with prosthesis donned. Eleven participants used an elastomeric liner with pin, two used a vacuum system, two wore a sleeve, and one utilized a lanyard system.

In the analysis below, data from channels monitoring between the patellar tendon and distal limb were included. Results for the posterior region are presented unless otherwise stated. Results from the anterior region were comparable to those from the posterior region unless otherwise stated.

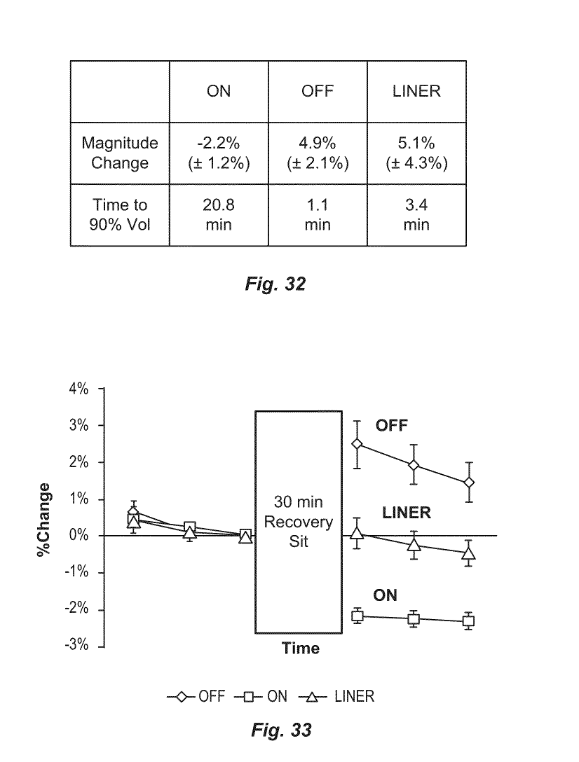

FIG. 31 shows the results percent volume change over the thirty minute time period for the ON, OFF, and LINER groups. FIG. 32 summarizes the results. For all sixteen participants, limb fluid volume reduced during the 30-minute recovery period with the socket donned (ON). On average the change in fluid volume was −2.2% (s.d.=1.2%, range −3.9% to −0.5%) of the initial limb fluid volume. All sixteen participants gained fluid volume during the recovery period when doffing the socket (OFF), averaging 5.5% (s.d.=2.5%, range 2.4% to 11.2%). Limb fluid volume change during the 30-minute sit with the socket doffed and liner donned (LINER) produced volume gains similar to removing the liner and prosthesis (OFF), with an average volume gain of 6.9% (s.d.=5.0%, range=−0.9% to 14.9%). Three subjects had difficulty getting their prosthesis back on after the OFF period.

The differences between volume gains in the three recovery conditions (ON, OFF, LINER) were statistically significant with p<0.001. A Bonferroni Comparison confirmed that volume gains during OFF were statistically elevated as compared to ON (p<0.001), and that volume gains during LINER were statistically elevated as compared to ON (p<0.01). A statistically significant difference did not exist between OFF and LINER volume change.

Accordingly, short-term fluid volume change, the difference in fluid volume after the fourth walking cycle (the first cycle after the 30 minute recovery period) minus that after the third walking cycle divided by that after the third walking cycle, was found to be negative for all sixteen participants during ON. Short-term fluid volume change during ON was on average −2.2% (s.d.=0.8%, range=−3.3%-−0.8%). During the OFF test, thirteen of sixteen participants showed fluid volume increase in the short-term. On average participants gained 2.45% (s.d.=2.5%, range=−2.0%-−8.2%) fluid volume in the short-term. Of the fourteen participants who completed the LINER protocol, eight showed short-term fluid volume gain while six showed short-term fluid volume loss. On average, participants gained 0.1% (s.d.=1.6%, range=−2.6%-−3.0%) fluid volume in the short-term during LINER.

FIG. 33 shows an average change in volume across all participants for each protocol, normalized to the 10 s stand after the third cycle. Standard error is shown as error bars. Short-term volume change is shown at time “a” while long-term volume change is shown at time “B”. Each point represents volume after one walk.

The repeated measures ANOVA of the short-term fluid volume change data showed statistically significant differences between the three sitting conditions (p=0.01), using the Greenhouse-Geisser adjustment for lack of sphericity. A Bonferroni analysis showed that differences between each test were statistically significant (p<0.01 in all cases). Therefore, the average change in volume between the fourth and third cycles likely depended on the recovery period condition (ON, OFF, LINER).

Long-term fluid volume change, the difference in fluid volume after the sixth walking cycle compared with that after the third walking cycle divided by that after the third walk cycle, for ON was −2.3% (s.d.=1.0%, range −4.6%-−0.8%). For OFF it was 1.5% (s.d.=2.1%, range −1.9% to 6.5%). For LINER it was −0.5% (s.d.=1.3%, range −3.0%-−0.9%). The repeated measures ANOVA for long-term fluid volume changes showed statistical significance between each of the three conditions (ON, OFF, LINER) with p=0.01. A Bonferroni analysis showed that differences between tests were each statistically significant (p<0.01 in all cases). Therefore, the average long-term volume change likely depended on the recovery period condition (ON, OFF, LINER).

Anterior and posterior results were comparable for all comparisons for ON and OFF. However, for LINER anterior and posterior results differed. Short-term anterior mean fluid volume changes were greater than posterior mean fluid volume changes for LINER. Anterior changes were 0.7% (s.d.=1.4) and posterior changes were 0.1% (s.d.=1.6). Long-term anterior means were also greater than posterior means, −0.2% (s.d.=1.0) for anterior and −0.5% (s.d.=1.3) for posterior.

Based from the results for anterior vs. posterior differences, the fitting of the liner may be important towards interface pressure magnitude. A liner of circumference less than the circumference of the residual limb would apply hoop stress and compress the residual limb, reducing the capability of the person to recover fluid after walking A study on one of the participants was conducted to compare interface pressures for the subject's normal liner vs. one that was one size too small. The subject was a healthy male of 65 years of age who had a limb amputation as a result of traumatic injury eight years prior. Using force sensing resistors (FSRs) positioned between the liner and residual limb lower interface pressures were measured with the normal liner than with the liner that was too small. Table 3 shows the results from the study.

TABLE 3
Liner pressures from a single participant.

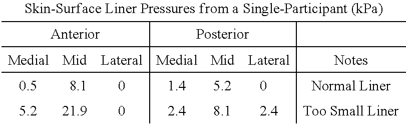

Normal Liner refers to the liner ordinarily worn by the participant. New liner was a new version of the normal liner that is artificially tight. The artificially tight liner was of the same thickness and manufacture as normal but one size smaller. Highlighted cells represent pressures high enough to occlude blood flow.

Discussion—

Use of socket release to accommodate residual limb volume loss may be an alternative to adding socks, air-filled or fluid-filled inserts inside the socket. With socket release, no material needs to be added inside the socket. The person using the prosthesis does not need to remember to carry socks or to bother with adjustment of settings on the prosthesis.

In summary patients following the ON protocol lost fluid volume during subsequent activity. Patients following the OFF protocol gained fluid volume. Patients following the LINER protocol resulted in fluid volumes between those of ON and OFF. The reason fluid volume recovery was greater during the rest period for OFF and LINER compared with ON may be two-fold. First, blood vessels within the residual limb expanded when interface pressures may be reduced for OFF and LINER, and thus more blood may fill the vasculature in the residual limb. Second, because interface pressures may be reduced upon doffing and thus interstitial pressures within the residual limb may be reduced, the balance may change between arterial-to-interstitial and interstitial-to-venous flow. Arterial-to-interstitial flow may increase and interstitial-to-venous flow may decrease resulting in an overall limb fluid volume increase.

Because fluid recovery within the residual limb may be maintained for both short and long term, it may be unlikely that the recovered fluid was exclusively blood within the vessels. If it had been only blood then that fluid would have quickly departed the residual limb upon subsequent weight bearing. Because that did not happen, fluid was likely recovered to the interstitial space during sit, and that fluid was not easily expelled upon subsequent weight-bearing.

The magnitude of fluid volume recovery during the 30-minute rest period was comparable between LINER and OFF, but the short and long-term changes were much lower for LINER than OFF. Behavior of the anterior region for the LINER condition was comparable to that for the posterior and anterior regions for the OFF condition.

Accordingly, socket release may be a good strategy to reduce residual limb fluid volume loss over the day. An amputee may benefit by removing the prosthesis during a lunch break, for example. Results illustrate that the degree of fluid volume recovery may be higher if the prosthesis is doffed right after activity compared with after resting. The optimal doffing duration may need to be tuned to the individual patient as the optimal interval might be different for different people. Participants prone to edema would need to be careful not to doff for too long because of the risk of difficulty redonning their prosthesis.

The greater the relief of interface pressure, the longer the recovered fluid is retained during subsequent activity. A person wearing a tight liner who keeps the liner on after doffing the prosthesis may not maintain as much fluid from socket release compared with wearing a loose liner or removing the liner altogether during socket release.

Automated socket release systems that adjust socket size at appropriate times rather than by user intervention may be advantageous. A socket may have fluid actuators that release based on posture and activity (using accelerometer and inclinometer sensor, for example). Towards this end the result from LINER in the present study may be accounted for. If the participant has a tight liner then even if the socket is released, fluid volume recovery might be minimal or non-existent. A loose liner may be an important criterion for effective socket release. Alternatively, a liner with a built in accommodation device (e.g., a filling bladder) may be beneficial.

Subjects gained volume over about the first 90 s after doffing. After 90 s it may be harmful to leave the socket on since it typically only induces limb fluid volume reduction. Thus a controller may be configured to adjust the socket to relieve pressures at least by 90 s. Results from this study set a time frame for release in group 2 subjects.

Recovery over the longer term may be higher if the liner is released. Thus hoop stress in the liner may need to be relieved.

If the liner is left on then the recovery may not be as long lasting. Therefore, tension in the liner may be relieved and/or volume of the liner may be reduced. Alternatively, socket shape may be relived with a loose liner. A tighter liner may restrict recovery due to liner hoop stress.

### Socket Shape Adjustments Based on Activity/Posture Research

To determine if limb volume was related to activity, and thus that gait could be used to determine needed socket volume adjustment, research was conducted to determine during what activities and postures fluid volume changes occurred on people with trans-tibial limb loss.

Methods—

Human subject volunteers were included in this study if they had a trans-tibial amputation at least 6 months prior, and at least 5 h/day were using a definitive, properly fitting prosthetic limb as deemed by the research prosthetist. Subjects needed to be capable of 90 s of continuous treadmill walking at a self-selected walking speed as well as 90 s of continuous standing. Residual limb length needed to be at least 9 cm so that electrodes could be properly spaced when placed on the residual limb. Subjects were not included if they had current skin breakdown, were unable to wear a prosthesis for at least 1.5 h continuously, and were unable to shift from standing to supine posture within 30 s (for vascular tests). All study procedures were approved by a University of Washington institutional review board, and informed consent was obtained before any study procedures were initiated.

On a separate day before biompedance testing but not more than 12 month prior, using techniques described in detail previously, the subjects were tested for presence of high blood pressure (orthostatic blood pressure (OBP)) and peripheral arterial disease (segmental limb pressures, ankle-brachial index (ABI)). An electronic blood pressure measurement unit (HEM-775, Omron, Kyoto, Japan) was used for OBP testing, and a commercial segmental limb pressure measurement system (TD312 Cuff Inflator, MV10 Manifold Selector, and SC12 and SC10 cuffs, Hokanson, Bellevue, Wash.) was used for segmental limb pressures and ABI assessment. Collected data were interpreted by a practicing endocrinologist using standard clinical procedures.

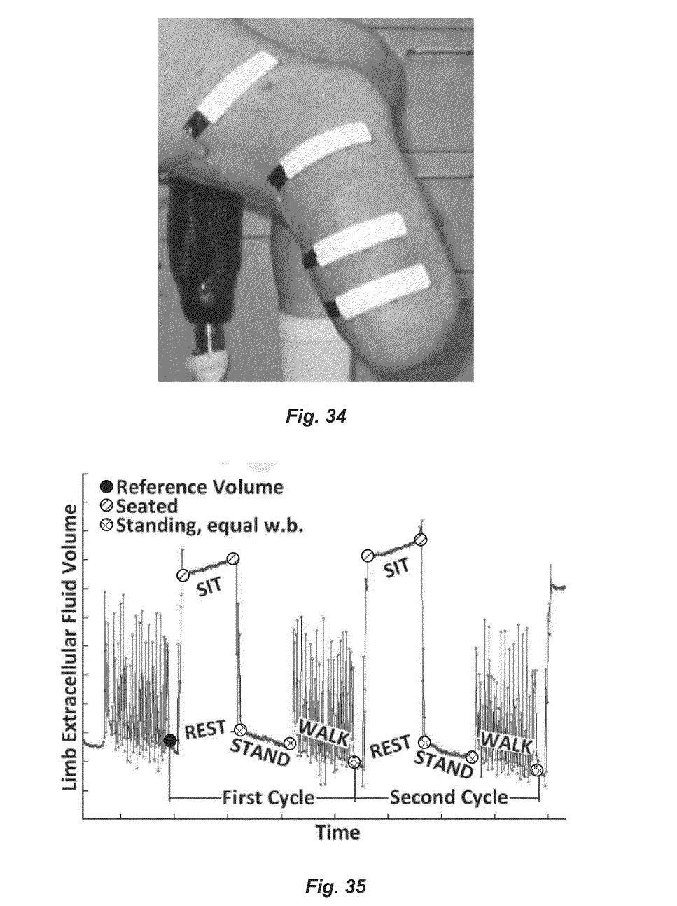

A commercial bioimpedance analyzer (XiTRON Hydra 4200, Impedimed, San Diego, Calif.) was modified to measure residual limb fluid volume on people with limb amputation. Electrical current between 100 and 700 μA was injected at 50 frequencies between 5 kHz and 1 MHz through current injection electrodes on proximal and distal aspects of the residual limb. FIG. 34 shows a residual limb with electrodes parallel with one another. Voltage was sensed with voltage sensing electrodes positioned between the two current-injecting electrodes. The current and voltage signals were demodulated within the XiTRON unit to calculate magnitude and phase difference for each frequency. The sampling rate of the XiTRON instrument was approximately 1 Hz.

Electrodes provided by the manufacturer were used (XiTRON, 77×20 mm contact surface, 0.81 mm thickness). A thin layer of ultrasonic coupling gel (Couplant D, GE Panametrics, West Chester, Ohio) was placed underside each electrode. Wires extending from the electrodes proximally to the XiTRON were strain relieved using Tegaderm (3M, St. Paul, Minn.). Outside the socket a custom connector with gold-plated pins (WPI Viking, Cooper Interconnect, Chelsea, Mass.) was used to connect the four electrodes to a coaxial cable that attached to the XiTRON unit with a robust connector (MS3116F106S, Burndy, Manchester, N.H.). Care was taken to ensure presence of the wires did not cause air channels to form that would have allowed air to escape from the limb-socket interface and induce a loss of suspension. The peak-to-peak fluctuation in the bioimpedance signal while the subject stood with equal weight bearing was less than 0.2% of the limb fluid volume.

After the subject arrived at the lab, the subject's mass and height were recorded. Then the subject sat for 10-15 minutes to achieve a homeostatic condition and answered questions about presence of diabetes or another major medical illness, and recent changes to their prosthesis. Then the subject doffed the prosthesis and the research practitioner inspected the subject's residual limb for soft tissue injury. She also evaluated if socket fit was acceptable for regular prosthesis use. If breakdown was present or socket fit was unacceptable then the test session was terminated and the subject was referred to his or her regular practitioner. Otherwise, the session was continued and sites where electrodes were to be placed were cleaned (Tracer Prep, 3M). Electrodes were placed on the limb such that all electrodes were parallel with each other. The proximal voltage sensing electrode was placed at the level of the patellar tendon on the posterior lateral surface of the limb proximal of the fibular head. The distal current injecting electrode was placed as far distally as possible but still on the cylindrical portion of the residual limb. The distal voltage sensing electrode was placed at least 3 cm proximal of the distal current injecting electrode. The proximal current injecting electrode was placed an average of 9 cm proximal to the proximal voltage electrode, outside of the socket under the proximal end of the elastomeric liner or sleeve suspension.

Continuous bioimpedance data collection was initiated. Bioimpedance data were viewed in approximately real time (1 to 3 s delay) using custom Matlab (Mathworks, Natick, Wash.) code that implemented a Cole model, similar to that used in the XiTRON post-processing program so that set up problems could be identified if they existed.

The subject then donned the prosthesis and rested in a chair for 90 s (REST). Care was taken to ensure a proper sitting posture during all rest periods. A proper sitting posture was characterized by relaxed legs and a knee flexion angle of approximately 130 degrees. Then the subject was asked to stand for 90 s with equal weight bearing (STAND) with the prosthetic limb supported by an electronic scale (349KLX Health-O-Meter, Pelstar, Alsip, Ill.). The electronic scale was embedded within a short platform so that it was flush with the surface. If the subject's weight bearing on the prosthesis deviated by more than 10% of half the body weight then the subject was asked to shift his or her weight accordingly. Then the subject moved onto a treadmill and walked for 90 s at a self-selected walking speed (WALK). The same speed was used for all WALK cycles in a session for each subject. The subject then returned to the scale to stand under equal weight bearing for approximately 10 s, and then sat down in the chair. The cycle of 90 s REST, 90 s STAND, 90 s WALK, and 10 s stand was then repeated four additional times. The five cycles took less than 25 min to complete. At the conclusion the subject sat down and doffed the prosthesis. Bioimpedance data collection was terminated and the electrodes were removed.

Demodulated data stored to disk from the XiTRON were converted to extracellular and intracellular fluid impedances using a Cole modeling strategy. An anatomical limb model was used to calculate extracellular fluid volume from the Cole model results and limb dimension measurements. Some subjects were found to require part of the first WALK cycle to adjust to the treadmill and accomplish a repeatable gait. Thus the first REST/STAND/WALK cycle was not included in analysis, and instead analysis started from the brief stand after completion of the first REST/STAND/WALK cycle. This point was defined as the reference point for the trial. The beginning and the end of each REST, STAND, and WALK phase within each of the four subsequent cycles were identified and labeled. Example data for 2½ cycles are shown in FIG. 35.

Fluid volume changes during each phase (REST, STAND, and WALK) of each of the last four cycles of REST/STAND/WALK were calculated. The REST change in each cycle was calculated as the fluid volume at the beginning of the subsequent STAND minus that during the previous brief stand after the previous WALK. The STAND change was the fluid at the end of the 90 s STAND minus that at the beginning of the 90 s stand. The WALK change was calculated as the fluid volume during the brief stand after WALK minus that at the end of the immediately prior STAND. Thus only data collected during standing with equal weight-bearing were used to calculate fluid volume changes during the three phases (REST, STAND, and WALK).

Fluid volume changes were also calculated during the TRANSITION (stand-to-sit plus sit-to-stand) and SIT parts of the REST phases. Subjects with peripheral arterial disease may experience relatively slow fluid movement in and out of their residual limb during resting. Therefore, they would be may have low fluid volume changes during TRANSITION and high fluid volume changes during SIT, a low TRANSITION-SIT difference. Subjects without peripheral arterial disease may have fast fluid transport thus would be expected to experience high fluid volume changes during TRANSITION and low fluid volume changes during SIT, a high TRANSITION-SIT difference.

SIT fluid volume change was calculated the fluid at the end of the SIT phase minus that at the beginning of the SIT phase (FIG. 35). TRANSITION fluid volume changes were quantified as any changes during REST that were not within SIT. TRANSITION was the SIT magnitude subtracted from the REST magnitude (FIG. 36). Thus TRANSITION was the sum of the fluid volume change both from sitting down and standing up. Stand-to-sit from sit-to-stand fluid volume changes were not separated because knee flexion differences between the stand condition compared with the sit condition might affect those results and thus confound interpretation.

The fluid volume changes during the last four cycles of each phase (REST, STAND, and WALK) as well as those for the subgroups of REST, i.e. TRANSITION and SIT, were summed for each subject over the test session. The data for the separate phases were summed so that results with total fluid volume changes over the session (TOTAL) could be compared. TOTAL fluid volume change was defined as the fluid volume during the brief stand after the last REST/STAND/WALK minus that after the first REST/STAND/WALK cycle. All data were expressed as a percentage change relative to the fluid volume measured during the brief stand after the first WALK cycle.

Results—

A total of 26 volunteers participated in the study. However, data from two subjects were not included in analysis because their residual limb lengths were outside of the calibration range acceptable for use of the bioimpedance instrument. Data from the remaining 24 subjects are presented below.

During test sessions with equal durations of resting, standing, and walking, do subjects lose the most fluid volume during walking? How do fluid volume changes during walking compare with those during standing and resting?

Table 4 shows the percentage fluid volume changes relative to initial fluid volume during different activities for all subjects.

TABLE 4

| Activity | Range | | Mean (s.d.) | | Median |
|---|---|---|---|---|---|
| REST _{(TR+SIT)} | −1.9 to | 5.7 | 1.0 | (2.2) | 0.4 |
| STAND | −5.4 to | −0.7 | −2.6 | (1.1) | −2.6 |
| WALK | −4.9 to | 5.7 | 1.0 | (2.5) | 1.3 |
| TOTAL | −2.7 to | 2.4 | −0.6 | (1.3) | −0.9 |

Analysis of fluid volume changes over the test session for the different activities (REST, STAND, and WALK) showed that the highest mean fluid volume loss was during STAND, averaging a 2.6% (s.d. 1.1%) loss over the test session. On average, subjects gained fluid volume during WALK and REST, with a mean limb fluid volume increase of 1.0% (s.d. 2.5%) during WALK and 1.0% (s.d. 2.2%) during REST. Fluid volume changes during STAND were significantly different from those during WALK (p<0.001), and from those during REST (p<0.001). REST and WALK fluid volume changes were not significantly different from each other (p=0.83).

Limb fluid volume changes for each subject were relatively consistent across the four cycles. Average coefficients of variation across the four cycles were 1.3 for REST, 0.2 for STAND, and 0.7 for WALK, where coefficient of variation was calculated using:

$$\begin{matrix} {Var}_{Avg} = \frac{{\sum }_{i = 1}^{n} Subject {Var}_{i}}{{n}_{subjects}} & ( 11 ) \\ Subject {Var}_{i} = \frac{SD ( Cycle 2 + Cycle 3 + Cycle 4 + Cycle 5 )}{Mean ( Cycle 2 + Cycle 3 + Cycle 4 + Cycle 5 )} & ( 12 )\end{matrix}$$

How much variability was there in the data across subjects? In other words did all subjects lose fluid volume during walking and standing and gain fluid volume during resting? Did all subjects who lost fluid volume during walking lose fluid volume over the entire test session? Was variability explained by subject characteristics?

All participants lost fluid volume during STAND. The rate of fluid volume loss within the STAND periods was calculated to investigate how rapidly fluid volume losses occurred. The average rate of change was −0.4%/min (s.d. 0.2).

Surprisingly, not all participants lost fluid volume during WALK. Eight participants lost fluid volume during WALK while sixteen gained. Surprisingly, not all participants gained fluid volume during REST. Fifteen participants gained fluid volume during REST while nine lost. All except three subjects experienced greater fluid volume losses during STAND than during WALK. All experienced greater fluid volume losses during STAND than during REST.

Table 5 shows the correlations between the various activities and with the percent total fluid volume change over the test session (TOTAL).

TABLE 5

| | REST | WALK | STAND |
|---|---|---|---|
| WALK | −0.81 | | |
| STAND | −0.41 | 0.03 | |
| Total | −0.26 | 0.63 | 0.22 |

Bold face in Table 5 indicates statistically significant results at the 0.05 level or smaller. The highest absolute value correlation between activities was for WALK and REST (0.81), followed by STAND and REST (0.41). A high absolute value correlation, close to 1.00, indicates a strong relationship between the variables. Thus in general, subjects who lost fluid volume during WALK gained fluid volume during REST. WALK was the only activity for which there was a statistically significant correlation with TOTAL (0.63). Only six individuals (25%) lost fluid volume for WALK and TOTAL (quadrant C), while thirteen gained in WALK but lost during the entire session (54.2%, quadrant D). However, linear regression analysis showed that percent WALK fluid volume change explained only 39.3% of the variation of TOTAL. The analysis of residuals showed that the model did not fit the data well. Therefore, percent WALK fluid volume change was not a good predictor of TOTAL fluid volume change.

To assess whether percent volume change was associated with gender, presence of peripheral arterial disease (PAD), high blood pressure (HBP), and diabetes, the distribution of percent fluid volume changes for all three activities were compared. There were no statistically significant differences in distribution for: males versus females for WALK (p=0.66), STAND ( [symbol] p=0.26), and REST (p=0.32); for individuals with and without PAD for WALK (p=0.91), STAND (p=0.25) and REST (p=0.49); and for individuals with and without diabetes for WALK (p=0.93), STAND (p=0.19), and REST (p=0.61). For presence of high blood pressure, there were no statistically significant differences in distributions of percentage fluid volume change between individuals with and without HBP for WALK (p=0.61) and REST (p=0.46), but the two groups were statistically different for STAND (p=0.015), where loss of fluid volume was larger among people without HBP (median=−2.8) when compared to people with HBP (median=−1.9).

Was there variability in the time course of fluid volume recovery during resting? Were differences explained by subject characteristics, including gender, presence of peripheral arterial disease, high blood pressure, and diabetes?

There was considerable variability in TRANSITION and SIT fluid volume changes among subjects. Table 6 shows the percentage fluid volume changes relative to initial fluid volume during TRANSITION and SIT for all subjects.

TABLE 6

| Activity | Range | Mean (s.d.) | | Median |
|---|---|---|---|---|
| TRANSITION | −5.1 to 5.3 | −0.2 | (2.5) | 0.0 |
| SIT | −1.4 to 4.9 | 1.1 | (1.6) | 0.7 |

Eleven subjects lost fluid volume during TRANSITION while thirteen gained. Five subjects lost fluid volume during SIT while nineteen gained. There was thus much variability in the time course of fluid volume recovery during REST.

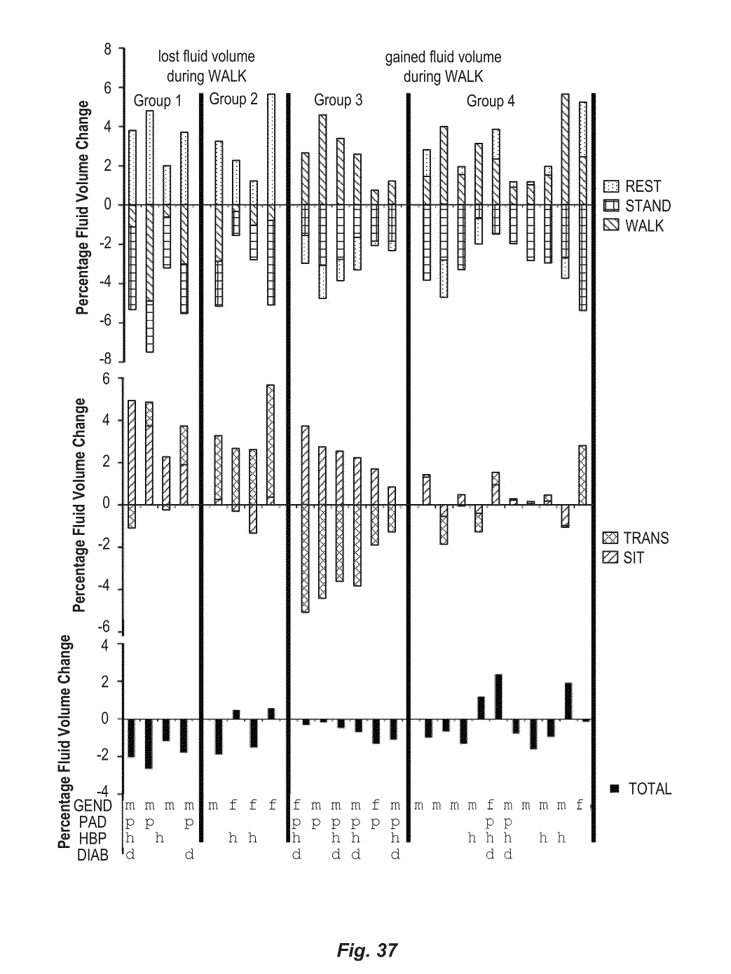

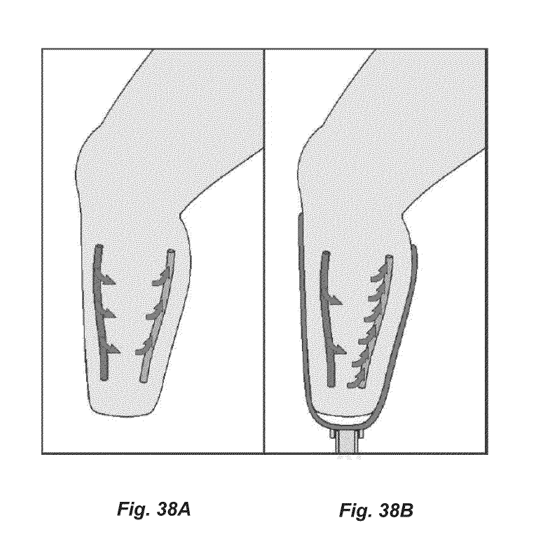

Extending from the above results, an exploratory analysis was conducted to determine if subjects with common TRANSITION/SIT/WALK fluid volume change patterns had common characteristics (STAND results were not included in the exploratory analysis since all subjects lost fluid volume during STAND). If they did then potentially subject characteristics could be used to predict fluid volume change patterns. Subjects were divided into those that lost fluid volume during WALK and those that gained. The basis for selecting direction of WALK fluid volume change as an initial delineator of subjects into sub-groups was prior experience that subjects who lost fluid volume during walk typically had health problems. Then within each of those two collections of participants, subject data was ordered from low to high TRANSITION-SIT differences. Subjects with low TRANSITION-SIT values may experience slow fluid transport and thus to be in poor health. Four groups emerged as shown in FIG. 37.
1st group—Subjects who lost fluid volume during WALK, gained during SIT, had relatively little fluid volume change during TRANSITION, and thus had low TRANSITION-SIT values. These subjects tended to have PAD.
2nd group—Subjects who lost fluid volume during WALK, had relatively little change during SIT, and gained fluid volume during TRANSITION. These subjects tended not to have PAD.
3rd group—Subjects who gained fluid volume during WALK and SIT but lost much fluid volume during TRANSITION. These subjects tended to have PAD.
4th group—Subjects who gained fluid volume during WALK but experienced relatively little change during SIT and TRANSITION. These subjects tended to not have PAD.

The data of FIG. 37 shows that, surprisingly, not all subjects lost fluid volume during walking Subjects classified in group 1 and group 2 lost fluid volume during walking while subjects classified in group 3 and group 4 gained fluid volume during walking FIG. 37 also shows that all subjects lost fluid volume during standing. Thus standing may be a major source of fluid volume loss and may have a greater influence on fluid volume than walking FIG. 37 also shows that not all subjects gained fluid volume during resting with the prosthesis donned. Subjects who lost fluid volume during walking, generally gained volume during resting. Thus resting with the prosthesis donned may contribute to a person's daily fluid volume loss.

Discussion—

Measurement of fluid volume changes during different activities of people with limb amputation may provide practitioners with insight useful towards prosthetic design and towards counseling their patients on when to expect fluid volume changes in their residual limb. The data may also provide information useful towards the design of control strategies to adjust prostheses to accommodate limb fluid volume fluctuations based upon the activity the prosthesis user is currently conducting.

Data used to calculate fluid volume changes during REST, STAND, and WALK were collected while the subject was in a consistent posture, standing with equal weight-bearing. This strategy ensured consistent knee flexion from trial to trial, a variable that might otherwise affect skin strains between the voltage-sensing electrodes and thus impact interpretation of the fluid volume measurement. Similarly, SIT fluid volume change was calculated with the subject in a consistent posture from the beginning to end of each SIT period, avoiding impact of change in knee flexion on the results. No subject complained about presence of the electrodes or wires.

During test sessions with equal durations of resting, standing, and walking, do subjects lose the most fluid volume during walking′? How do fluid volume changes during walking compare with those during standing and resting?

STAND was the dominant source of fluid volume lost as illustrated by the results presented in Table 4. The result that most of the fluid volume losses over the session occurred during STAND is consistent with physical and physiologic constraints experienced by a residual limb within a prosthetic socket. During STAND, because the limb is enclosed by the socket, pressures applied at the limb-socket interface to support weight bearing increase the pressure within the interstitial space inside the residual limb. This increased interstitial fluid pressure causes interstitial-to-venous fluid transport to dominate over arterial-to-interstitial fluid transport, illustrated in FIG. 38A-38B. There may be no compensatory physiological mechanism during STAND, for example no intermittent pressure release, that counters this imbalance. The result for all participants is thus a gradual fluid volume loss.

How much variability was there in the data across subjects? In other words did all subjects lose fluid volume during walking and standing and gain fluid volume during resting? Did all subjects who lost fluid volume during walking lose fluid volume over the entire test session? Was variability explained by subject characteristics?

Sixteen of the 24 subjects gained fluid volume during WALK. For these subjects WALK fluid volume gains countered fluid volume losses during STAND. Several possible mechanisms may be at work during WALK to accomplish fluid volume gains: the muscle in the residual limb actively pumping fluid into the residual limb during swing phase; the elevated arterial pressure, increasing arterial fluid drive; and proximal displacement of the limb in the socket during swing phase to release pressures intermittently and thus facilitate fluid volume return. It is also possible that the immediately prior STAND period in the protocol temporarily dehydrated the residual limb and served to accentuate the subsequent WALK fluid volume gains.

Based from clinical experience, WALK was expected to have the greatest impact on TOTAL fluid volume change. However, unexpectedly and surprisingly, the results showed that WALK explained only 35% of TOTAL. The results counter clinical expectation. Clinicians may need to consider other activities besides walking when helping a patient to understand what causes limb fluid volume change and predict their daily volume fluctuation.

Further, it was unexpected and surprising that some subjects lost limb fluid volume during REST. REST was previously considered a time of recovery for all subjects. However, for a person using a prosthesis, the pressure applied by the liner and socket even while sitting can increase interstitial fluid pressure and cause volume loss. In a recent study on 16 amputee subjects, after walking and then sitting down in a chair subjects lost an average of 2.2% limb fluid volume over a 30 min period. The moderate correlation between REST and WALK (−0.81) suggests that to some extent, it can be predicated when a person who loses a lot of fluid volume during WALK will gain a lot during REST. Subject education about doffing or releasing the socket when a long sitting interval is anticipated might be helpful towards stabilizing limb fluid volume.

Was there variability in the time course of fluid volume recovery during resting? Were differences explained by subject characteristics, including gender, presence of peripheral arterial disease, high blood pressure, and diabetes?

While subjects with peripheral arterial disease may be expected to be the only subjects to demonstrate fluid volume losses during WALK, it was found that only half of the subjects who lost fluid volume during WALK had PAD (four of eight (including one subject on lifetime antibiotics)). Those four subjects who lost fluid volume during WALK but were healthy, however, did show one noticeable difference compared to the four subjects with PAD. They experienced high TRANSITION fluid volume increases thus quickly moved fluid into and out of the residual limb during REST, unlike PAD subjects.

This difference in behavior during REST between healthy participants (Group 2) and subjects with PAD (Group 1) may be relevant to prosthetic fitting because it suggests different accommodation strategies should be used to facilitate fluid volume recovery during REST. Following the strategy of accentuating those actions with fluid volume gains, using a socket release strategy, where the volume of the socket is temporarily enlarged while the subject rests, either through doffing or an automated socket-release mechanism, participants with PAD (group 1) may benefit from a relatively long release interval to facilitate fluid volume recovery. Subjects without PAD (group 2) may require only a short release interval. Too long a release interval for subjects without PAD (group 2) might create an edematous limb, making it difficult to re-don the prosthesis when the prosthesis user returns to weight-bearing. Further, subjects without PAD (group 2) who lost fluid volume during WALK may be expected to respond well to suction sockets or elevated-vacuum technology. Because they have the capability to recover limb fluid volume quickly, these subjects might accentuate their limb fluid volume recovery during swing phase if suction or elevated vacuum is applied.

Though most participants compensated for fluid volume loss during STAND by increasing fluid volume during WALK, some of them experienced so much fluid volume loss during REST that their overall fluid volume change was negative (3rd group). Most of these participants had PAD as shown in FIG. 37. Interestingly, these subjects lost considerable volume during TRANSITION, not during SIT. This result suggests that fluid that was gained during SIT was easily displaced out of the limb upon rising for the subsequent STAND. Because of the quick expulsion of fluid upon standing, the fluid may be primarily blood within the vascular network as opposed to fluid within the interstitial space. An effective accommodation strategy for these subjects would reduce TRANSITION fluid volume loss, possibly through a quick-release mechanism that relieved interface pressures immediately upon sitting, combined with prolonged socket release during resting so as to maximize SIT fluid recovery.

The two outlier subjects with PAD in FIG. 37 (5th and 6th from right) may have also had venous insufficiency, offsetting reduced arterial-to-interstitial transport with reduced interstitial-to-venous transport. Presence of venous insufficiency may be difficult to evaluate because venous insufficiency often is not systemic but instead localized. A subject might not demonstrate presence of venous insufficiency in the contralateral limb using standard test methods (ASGP testing) but may still have venous insufficiency in the residual limb.

For subjects in the 4th group (FIG. 37), fluid volumes changes over the test session were dominated by differences between STAND and WALK fluid volume changes. If these subjects spent more time walking and less time standing, possibly their daily fluid volume loss would decrease. These subjects may benefit from practitioner education on this topic so as to help them better understanding, predict, and accommodate their daily limb fluid volume change.

It is worth noting that our sitting interval in this protocol was only 90 s long. 30-minute sitting periods with the prosthesis donned may lead to a gradual loss in fluid volume in subjects that starts about 90 s after sitting down. Thus when sitting is the main or only source of a patient's fluid volume gain (1st and 3rd groups in FIG. 37), the practitioner and patient may need to be conscientious of the time durations the patient sits. It might be that if these participants sat for periods longer than 90 s then the benefits of resting towards increasing limb fluid volume would be reduced. Socket pressure release may counteract fluid volume loss during sitting.

Subjects with high TRANSITION values were mainly women. It has been noted in physiology literature that women, in general, do not empty their veins as rapidly as men. So when socket pressures are released (e.g., transitioning from standing to sitting) interstitial fluid levels may increase dramatically within the residual limb. This change would happen because of the slow capability to empty the veins, limiting limb fluid outflow, and thus may explain why subjects with high TRANSITION values were mainly women. The single female subject who did not demonstrate these trends was the only female subject who had PAD. Possibly her arterial occlusion offset her limited venous outflow so that she maintained good limb fluid balance.

Conclusion—

During test sessions with equal intervals of resting, standing, and walking activities: walking did not dominate total volume loss over the session. Instead, standing dominated fluid volume loss, average a rate of −0.43%/min. All participants lost fluid volume during STAND. Sixteen participants gained fluid volume during walking while eight lost fluid volume during walking. All subjects who lost fluid volume during walking gained fluid volume during resting. However, not all subjects who gained fluid volume during walking lost fluid volume during rest. The correlation between WALK and REST fluid volume change was −0.81. There were no significant difference changes in distribution of fluid volume change during REST, STAND, or WALK for male vs. female, etc. Subjects with PAD had larger fluid volume gains during SIT than subjects without PAD. In general, subjects with low TRANS-SIT values, indicative of slow fluid transport, had PAD while subjects with high TRANS-SIT values did not. However, TRANS-SIT not related to TOTAL. Diagnosis of each person's profile through a clinical test may facilitate clinical treatment.

What is claimed is:

1. A method for customizing a prosthetics accommodation device for a residual limb of a prosthetics user, the method comprising:
identifying an activity volume profile of the prosthetics user, the activity volume profile of the prosthetics user corresponding to a residual limb fluid volume response to prosthetics user activity;
customizing a controller of the prosthetics accommodation device to provide customized automated accommodation for the prosthetics user based on the identified activity volume profile and based on detected prosthetics user activity.

2. The method of claim 1, wherein the prosthetics accommodation device comprises a bladder accommodation device.

3. The method of claim 2, wherein the bladder accommodation device is incorporated within a liner for the residual limb of the prosthetics user.

4. The method of claim 1, wherein the prosthetics accommodation device comprises a vacuum assist device.

5. The method of claim 1, wherein the prosthetics accommodation device is incorporated within an insert placed into the prosthesis.

6. The method of claim 1, wherein the activity volume profile of the prosthetics user corresponds to the residual limb fluid volume response to prosthetics user walking, sitting, and standing.

7. The method of claim 6, wherein the residual limb fluid volume response includes residual limb fluid volume response to prosthetics user transitions between standing and sitting.

8. The method of claim 1, wherein the activity volume profile indicates that the prosthetic user loses fluid volume during walking and gains fluid volume during sitting, and wherein the controller is customized to provide prolonged socket pressure release during detected prosthetic user sitting and/or resting after walking, the prolonged socket pressure release ranging from 3 min to 16 hours.

9. The method of claim 1, wherein the activity volume profile indicates that the prosthetic user loses fluid volume during walking and gains fluid volume during stand-sit/sit-stand transitions, and wherein the controller is customized to provide vacuum assist during detected prosthetic user walking.

10. The method of claim 1, wherein the activity volume profile indicates that the prosthetic user gains fluid volume during walking, loses fluid volume during stand-sit/sit-stand transitions, and gains fluid volume during sitting, and wherein the controller is customized to provide socket immediate pressure release and prolonged pressure release during detected prosthetic user sitting and/or resting after walking, the immediate pressure release occurring within 2 seconds to within 20 seconds of detection of prosthetic user sitting and/or resting after walking and the prolonged pressure release ranging from 3 min to 16 hours.

11. The method of claim 1, wherein the activity volume profile indicates that the prosthetic user gains fluid volume during walking, but experiences less than 1% volume change during resting, and wherein the controller is customized to provide prosthetic user alerts, the alerts provided when prosthetic user standing of more than 5 minutes is detected.

12. The method of claim 11, wherein the controller is customized to provide prosthetic user alerts when prosthetic user standing of more than 1 minute is detected.

13. The method of claim 1, wherein the prosthetics user activity is detected by a single 3-axis accelerometer.

14. The method of claim 1, wherein the prosthetics user activity of user donning and doffing the prosthetic is detected by a socket proximity sensor.

15. The method of claim 14, wherein the socket proximity sensor comprises an infrared distance sensor or an ultrasonic distance sensor.

16. The method of claim 1, wherein the controller is further customized to automatically adjust the prosthetics accommodation device based on a position of the residual limb within the prosthetic socket.

17. The method of claim 16, wherein the controller compares an actual high position and an actual low position of the residual limb within the prosthetic socket to an upper target and a lower target in order to adjust the accommodation device.

18. The method of claim 17, wherein the controller increases fit when the actual high position>upper target>actual low position>lower target; and
wherein the controller decreases pressure with the accommodation device when the upper target>actual high position>lower target>actual low position.

19. The method of claim 17, wherein the position of the residual limb in the prosthetic socket is indicated by signals from a piezoresistive force sensor.

20. The method of claim 1, wherein the controller is configured to operatively couple with a user input device, the user input device configured to send a signal to the controller indicating that the prosthetics user intends to stand and/or walk from a sitting position; and
wherein the controller is configured to adjust the prosthetics accommodation device in anticipation for the standing and/or walking of the prosthetics user.

21. The method of claim 1, wherein the prosthetics accommodation device comprises one or more pressure pulse sensors and wherein the controller is configured to be operatively coupled to the one or more pressure pulse sensors to adjust a fluid volume recovery strategy or one or more stress locations of the prosthetics accommodation device in response to the one or more pressure pulse sensors.

22. A control system for use with a prosthetics accommodation device to provide customized accommodation for a residual limb of a prosthetics user, the control system comprising:
one or more sensors configured to generate signals indicative of prosthetics user activity;
a controller operatively coupled to the one or more sensors and configured to be operatively coupled to the prosthetics accommodation device, the controller further configured to:
interpret the signals from the one or more sensors to determine current activities of the prosthetics user;
receive customization input, the customization input indicative of an activity volume profile of the prosthetics user, the activity volume profile of the prosthetics user corresponding to a residual limb fluid volume response to prosthetics user activity; and
actuate the prosthetics accommodation device to provide customized accommodation for the prosthetics user based on the customization input and based on the signals from the one or more sensors that are indicative of prosthetics user activity.

23. The control system of claim 22, wherein the prosthetics accommodation device comprises a bladder accommodation device.

24. The control system of claim 23, wherein the bladder accommodation device is incorporated within a liner for the residual limb of the prosthetics user.

25. The control system of claim 22, wherein the prosthetics accommodation device comprises a vacuum assist device.

26. The control system of claim 22, wherein the prosthetics accommodation device is incorporated within an insert placed into the prosthesis.

27. The control system of claim 22, wherein the activity volume profile of the prosthetics user corresponds to the residual limb fluid volume response to prosthetics user walking, sitting, and standing.

28. The control system of claim 27, wherein the residual limb fluid volume response includes residual limb fluid volume response to prosthetics user transitions between standing and sitting.

29. The control system of claim 22, wherein the controller actuates the prosthetics accommodation device to provide prolonged socket pressure release during detected prosthetic user sitting and/or resting after walking when the customization input indicates an activity volume profile where the prosthetic user loses fluid volume during walking and gains fluid volume during sitting, the prolonged socket pressure release ranging from 3 min to 16 hours.

30. The control system of claim 22, wherein the controller provides vacuum assist during detected prosthetic user walking when the customization input indicates an activity volume profile where the prosthetic user loses fluid volume during walking and gains fluid volume during stand-sit/sit-stand transitions.

31. The control system of claim 22, wherein the controller is customized to provide socket immediate pressure release and prolonged pressure release during detected prosthetic user sitting and/or resting after walking when the customization input indicates an activity volume profile where the prosthetic user gains fluid volume during walking, loses fluid volume during stand-sit/sit-stand transitions, and gains fluid volume during sitting, the immediate pressure release occurring within 2 seconds to within 20 seconds of detection of prosthetic user sitting and/or resting after walking and the prolonged pressure release ranging from 3 min to 16 hours.

32. The control system of claim 22, wherein the controller is customized to provide prosthetic user alerts to reduce prosthetic user standing when the customization input indicates an activity volume profile where the prosthetic user gains fluid volume during walking, but experiences less than 1% volume change during resting, the alerts provided when the system detects that the prosthetic user has stood for more than 5 minutes.

33. The control system of claim 32, wherein the controller is customized to provide prosthetic user alerts when the system detects that the prosthetic user has stood for more than 1 minute.

34. The control system of claim 22, wherein the prosthetics user activity is detected by a single 3-axis accelerometer.

35. The control system of claim 22, wherein the prosthetics user activity of user donning and doffing the prosthetic is detected by a socket proximity sensor.

36. The control system of claim 35, wherein the socket proximity sensor comprises an infrared distance sensor or an ultrasonic distance sensor.

37. The control system of claim 22, wherein the controller is further customized to automatically adjust the prosthetics accommodation device based on a position of the residual limb within the prosthetic socket.

38. The control system of claim 37, wherein the controller compares an actual high position and an actual low position of the residual limb within the prosthetic socket to an upper target and a lower target in order to adjust the accommodation device.

39. The control system of claim 38, wherein the controller increases fit with the accommodation device when the actual high position>upper target>actual low position>lower target; and
wherein the controller decreases pressure with the accommodation device when the upper target>actual high position>lower target>actual low position.

40. The control system of claim 38, wherein the position of the residual limb in the prosthetic socket is indicated by signals from a piezoresistive force sensor.

41. The control system of claim 22, wherein the controller is configured to operatively couple with a user input device, the user input device configured to send a signal to the controller indicating that the prosthetics user intends to stand and/or walk from a sitting position; and wherein the controller is configured to adjust the prosthetics accommodation device in anticipation for the standing and/or walking of the prosthetics user.

42. The control system of claim 22, wherein the prosthetics accommodation device comprises one or more pressure pulse sensors and wherein the controller is configured to be operatively coupled to the one or more pressure pulse sensors to adjust a fluid volume recovery strategy or one or more stress locations of the prosthetics accommodation device in response to signals from the one or more pressure pulse sensors.

43. A prosthetic device for a residual limb of a prosthetic user, the prosthetic device comprising:
an infrared sensor for detecting a presence of a residual limb;
a controller operatively coupled with the infrared sensor, the controller configured to automatically adjust a prosthetics accommodation device in response to detected prosthetics user activity, and wherein the controller is configured to recognize when the prosthetic user dons and doffs the prosthetic device in response to signals received from the infrared sensor.

44. A prosthetic device for a residual limb of a prosthetic user, the prosthetic device comprising:
a controller configured to automatically adjust a prosthetic accommodation device in response to detected user activity;
a padding configured to bear weight from the residual limb of the prosthetic user;
a force sensor under the foam padding and operatively coupled to the controller, the force sensor configured to output a signal to the controller that is indicative of a displacement distance of the padding in response to weight bearing of the residual limb of the prosthetic user;
the controller configured to adjust the prosthetic accommodation device in response to the displacement distance signal from the force sensor.
